(19) United States
(12) Patent Application Publication
(10) Pub. No.: US 2025/0270522 A1
(43) Pub. Date: Aug. 28, 2025

(54) ISOLATED TRANSPOSASE AND USE THEREOF

(71) Applicant: Beijing AstraGenomics Technology Co., Ltd., Beijing (CN)

(72) Inventors: Chen ZHAO, Beijing (CN); Daqi YU, Beijing (CN); Ting WEI, Beijing (CN); Chengxi SHI, Beijing (CN)

(21) Appl. No.: 18/866,410

(22) PCT Filed: Mar. 12, 2024
(86) PCT No.: PCT/CN2024/081126

§ 371 Date: Nov. 15, 2024

(30) Foreign Application Priority Data
Mar. 27, 2023 (CN) 202310304787.X

(51) Int. Cl. C12N 9/12; C12N 15/90

(52) U.S. Cl. CPC C12N 9/1241; C12N 15/90; C12N 2800/90

(57) ABSTRACT

Provided are an isolated transposase and the use thereof. Provided also are a nucleic acid and a nucleic acid construct encoding the transposase, a nucleic acid set and a nucleic acid set construct, and a composition, a recombinant vector, a recombinant host cell and a kit comprising the transposase. Provided also are a method for introducing an exogenous nucleic acid fragment into the genome of a host cell, a method for editing the genome of a host cell, and a method for obtaining a host cell containing an exogenous nucleic acid fragment in the genome. Provided also are the use of the transposase, the nucleic acid and the nucleic acid construct, the nucleic acid set and the nucleic acid set construct, the composition, the recombinant vector, or the recombinant host cell for introducing an exogenous nucleic acid fragment gene into the genome of a host cell or preparing a drug or a preparation for gene therapy, cell therapy, genome research, or stem cell induction and post-induction differentiation.

### CROSS-REFERENCE TO RELATED APPLICATIONS

This application claims priority to Chinese Patent Application No. 202310304787X, filed with the China National Intellectual Property Administration on Mar. 27, 2023, the entire contents of which are hereby incorporated by reference in their entirety for all purpose.

### TECHNICAL FIELD

The present application relates to the field of molecular biology, and specifically to an isolated transposase and the use thereof. The present application further specifically relates to: a nucleic acid and a nucleic acid construct encoding the transposase, a nucleic acid set and a nucleic acid set construct, and a composition, a recombinant vector, a recombinant host cell and a kit comprising the transposase. The present application further specifically relates to: a method for introducing an exogenous nucleic acid fragment into the genome of a host cell, a method for editing the genome of a host cell, and a method for obtaining a host cell containing an exogenous nucleic acid fragment in the genome. The present application further specifically relates to the use of the transposase, the nucleic acid and the nucleic acid construct, the nucleic acid set and the nucleic acid set construct, the composition, the recombinant vector, or the recombinant host cell for introducing an exogenous nucleic acid fragment gene into the genome of a host cell or preparing a drug or a preparation for gene therapy, cell therapy, genome research, or stem cell induction and post-induction differentiation.

### BACKGROUND

A transposon is a DNA sequence that can be inserted into or excised from the genome to transfer its own sequence or a complete copy of its own sequence within or between genomes. Transposons fall into two main categories, which are referred to herein primarily as type II transposons (DNA transposons), consisting of a terminal inverted repeat (TIR) at both ends and a gene encoding a transposase. Transposons have a “cut-and-paste” transposition mechanism, where DNA is cleaved from chromosomes and directly inserted into other parts of the genome.

Transposases are sequence-specific DNA-binding proteins expressed by DNA transposon sequences, comprising catalytic domains that mediate DNA breakage and ligation. Transposases can recognize and bind to TIRs at both ends of transposons, forming a bulge complex, and then remove the DNA transposon from the original site and integrate it into a new site. The transposition activity of a transposon is mainly dependent on the expression level and activity of transposases. Therefore, DNA transposons having a high transposase activity are a major requirement for the development of transposon function-based gene editing tools.

Gene insertion and integration of large fragments have important application value in fields such as gene therapy, molecular breeding of animals and plants, and engineering of industrial microorganisms. Currently, there is a lack of effective tools and systems for insertion and integration of a large fragment gene in the industry. In recent years, the scientific community has developed some tools and methods capable of inserting and integrating a large fragment gene, but these methods still have some problems. For example, in cellular immunotherapy and gene therapy for genetic diseases, lentivirus or retrovirus are most commonly used to integrate gene sequences, and based on this, there are several therapeutic products for the treatment of tumors and genetic diseases (Aiuti, A., Roncarolo, M. G. and Naldini, L. (2017) Gene therapy for ADA-SCID, the first marketing approval of an ex vivo gene therapy in Europe: paving the road for the next generation of advanced therapy medicinal products. EMBO Mol. Med. 9, 737-740; Aiuti, A. et al. (2009) Gene therapy for immunodeficiency due to adenosine deaminase deficiency. N. Engl. J. Med. 360, 447-458). However, using viruses to integrate a large fragment gene has some potential application limitations: first, the randomness of virus integration in the genome creates the risk of cancer; second, the size of an exogenous gene the virus can carry is also limited, which is not conducive to the transfer of a therapeutic large fragment gene; third, the immunogenicity of the virus may affect the long-term expression of an exogenous therapeutic gene and re-administration; fourth, the production of viruses needs to be completed with the help of living cells, which makes the quality control and downstream processing of such products more complex and more expensive, and has certain disadvantages in terms of industrialization. Therefore, non-viral large fragment integration can avoid various disadvantages caused by viral integration and become a valuable tool in gene therapy.

As a non-viral gene integration tool, DNA transposons not only can achieve the integration in a host genome and stable expression of a large fragment of an exogenous gene, but also can circumvent negative effects such as immunogenicity, and thus some transposons have been used in gene therapy. Although transposons have been proved to be widely present in various fields from prokaryotes to eukaryotes, during evolution, in order to maintain genomic stability, a large number of transposon fragments become silently inactive. At present, a few highly active and valuable transposon tools, such as Sleeping Beauty (SB), PiggyBac (PB) and Tol2, are used in gene therapy studies. Therefore, the excavation of more highly active transposon tools and the verification and detection of their functions can provide more, better and flexible choices for the development of gene therapy strategies.

It should be noted that methods described in this section are not necessarily methods that have been previously conceived or employed. It should not be assumed that any of the methods described in this section is considered to be the prior art just because they are included in this section, unless otherwise indicated expressly. Similarly, the problem mentioned in this section should not be considered to be universally recognized in any prior art, unless otherwise indicated expressly.

### SUMMARY

Based on this, in order to seek more advanced and more effective non-viral gene integration tools, the present application provides an isolated transposase, wherein the transposase has a transposase sequence selected from the following (i) or a variant sequence of the aforementioned transposase having a transposase activity in (ii)-(iv): (i) at least one amino acid sequence as shown in any one of SEQ ID NOs: 1-79; (ii) at least one of sequences obtained by performing deletion, substitution, insertion, or mutation of 1, 2, 3, 4, 5, 6, 7, 8, 9 or 10 amino acids on the amino acid sequence as shown in any one of SEQ ID NOs: 1-79; (iii) at least one of amino acid sequences having at least 70%, 80%, 90%, 95% or 99% identity to the amino acid sequence as shown in any one of SEQ ID NOs: 1-79; and (iv) at least one of sequences obtained by further fusing the amino acid sequence as shown in any one of SEQ ID NOs: 1-79 with other sequences. The transposase provided in the present application has comparable or even higher transposition activity compared to Sleeping Beauty (SB) and PiggyBac (PB), which are widely used now, providing more or better choices for the development of gene integration tools.

According to an embodiment of the present application, an isolated transposase can be provided, wherein the transposase comprises an amino acid sequence as shown in formula (I)

DE (I),

wherein D is aspartic acid; and E is glutamic acid.

According to an embodiment of the present application, an isolated transposase can be provided, wherein the transposase comprises an amino acid sequence as shown in formula (II):

D(X₁)ₐH (II),

wherein D is aspartic acid; H is histidine; a is the number of amino acids; and (X₁) is any amino acid, and a is 5.

According to an embodiment of the present application, an isolated transposase can be provided, wherein the transposase comprises an amino acid sequence as shown in formula (III):

P(X₂)(X₃) (III),

wherein P is proline; X₂ is any amino acid; and X₃ is aspartic acid or glutamic acid.

According to an embodiment of the present application, an isolated transposase can be provided, wherein the transposase comprises at least two of the amino acid sequences as shown in formula (I), formula (II), and formula (III).

According to an embodiment of the present application, an isolated transposase can be provided, wherein the transposase comprises the amino acid sequences as shown in formula (I), formula (II), and formula (III).

According to an embodiment of the present application, a nucleic acid can be provided, wherein, the nucleic acid encodes the transposase described in the present application.

According to an embodiment of the present application, a nucleic acid construct can be provided, comprising the nucleic acid according to the present application, and further comprising a promoter.

According to an embodiment of the present application, a nucleic acid set can be provided, wherein the 5′ recognition sequence comprises at least one of the nucleotide sequences as shown in SEQ ID NOs: 80-158.

According to an embodiment of the present application, a nucleic acid set can be provided, wherein the 3′ recognition sequence comprises at least one of the nucleotide sequences as shown in SEQ ID NOs: 159-237.

According to an embodiment of the present application, a nucleic acid set can be provided, comprising a 5′ recognition sequence and a 3′ recognition sequence, wherein the 5′ recognition sequence comprises the nucleotide sequence as shown in any one of SEQ ID NOs: 80-158 or a variant thereof, the 3′ recognition sequence comprises the nucleotide sequence as shown in any one of SEQ ID NOs: 159-237 or a variant thereof, and the nucleic acid set can be recognized by a specific transposase.

According to an embodiment of the present application, a nucleic acid set construct can be provided, the nucleic acid set construct includes the nucleic acid set described in the present application and further includes an exogenous nucleic acid fragment.

According to an embodiment of the present application, a composition may be provided, wherein, the composition includes: a Tc1/mariner superfamily transposase or a functional fragment thereof, or a nucleic acid encoding the Tc1/mariner superfamily transposase or the functional fragment thereof, wherein the transposase or the functional fragment thereof has a function of catalyzing the insertion of an exogenous nucleic acid fragment into the genome of a cell; and a nucleic acid set, wherein the nucleic acid set can be recognized by a specific transposase or a functional fragment thereof.

According to an embodiment of the present application, a recombinant vector can be provided, wherein, the recombinant vector comprises the nucleic acid encoding the transposase described in the present application, the nucleic acid described in the present application, the nucleic acid construct described in the present application, the nucleic acid set described in the present application, the nucleic acid set construct described in the present application, or the composition described in the present application.

According to an embodiment of the present application, a recombinant host cell can be provided, wherein, the recombinant host cell comprises the transposase described in the present application, the nucleic acid encoding the transposase described in the present application, the nucleic acid described in the present application, the nucleic acid construct described in the present application, the nucleic acid set described in the present application, the nucleic acid set construct described in the present application, the composition described in the present application, or the recombinant vector described in the present application.

According to an embodiment of the present application, a method for introducing an exogenous nucleic acid fragment into the genome of a host cell can be provided, wherein, the method comprises: delivering the transposase described in the present application, the nucleic acid encoding the transposase described in the present application, the nucleic acid described in the present application, the nucleic acid construct described in the present application, the nucleic acid set described in the present application, the nucleic acid set construct described in the present application, the composition described in the present application, or the recombinant vector described in the present application into a host cell.

According to an embodiment of the present application, a method for editing the genome of a host cell can be provided, wherein, the method comprises: delivering the transposase described in the present application, the nucleic acid encoding the transposase described in the present application, the nucleic acid described in the present application, the nucleic acid construct described in the present application, the nucleic acid set described in the present application, the nucleic acid set construct described in the present application, the composition described in the present application, or the recombinant vector described in the present application into a host cell.

According to an embodiment of the present application, a method for obtaining a host cell containing an exogenous nucleic acid fragment in the genome can be provided, wherein, the method comprises: delivering the transposase described in the present application, the nucleic acid encoding the transposase described in the present application, the nucleic acid described in the present application, the nucleic acid construct described in the present application, the nucleic acid set described in the present application, the nucleic acid set construct described in the present application, the composition described in the present application, or the recombinant vector described in the present application into a host cell.

According to an embodiment of the present application, the use of the transposase described in the present application, the nucleic acid encoding the transposase described in the present application, the nucleic acid described in the present application, the nucleic acid construct described in the present application, the nucleic acid set described in the present application, the nucleic acid set construct described in the present application, the composition described in the present application, the recombinant vector described in the present application, or the recombinant host cell described in the present application for introducing an exogenous nucleic acid fragment into the genome of a host cell can be provided.

According to an embodiment of the present application, the use of the transposase described in the present application, the nucleic acid encoding the transposase described in the present application, the nucleic acid described in the present application, the nucleic acid construct described in the present application, the nucleic acid set described in the present application, the nucleic acid set construct described in the present application, the composition described in the present application, the recombinant vector described in the present application, or the recombinant host cell described in the present application for preparing a drug or a preparation for gene therapy, cell therapy, genome research, or stem cell induction and post-induction differentiation can be provided.

According to an embodiment of the present application, a kit can be provided, wherein, the kit comprises the transposase described in the present application, the nucleic acid encoding the transposase described in the present application, the nucleic acid described in the present application, the nucleic acid construct described in the present application, the nucleic acid set described in the present application, the nucleic acid set construct described in the present application, the composition described in the present application, the recombinant vector described in the present application, or the recombinant host cell described in the present application.

It should be understood that the content described in this section is not intended to identify critical or important features of the examples of the present application, and is not used to limit the scope of the present application. Other features of the present application will be easily understood through the following description.

### BRIEF DESCRIPTION OF THE DRAWINGS

The accompanying drawings exemplarily show embodiments and form a part of the specification, and are used to explain exemplary implementations of the embodiments together with a written description of the specification. The embodiments shown are merely for illustrative purposes and do not limit the scope of the claims. Throughout the accompanying drawings, the same reference numerals denote similar but not necessarily same elements.

FIG. 1 shows a schematic diagram of two plasmid vectors in the transposon activity detection system in example 1. Plasmid 1 is a plasmid expressing a transposase (Tn), and plasmid 2 is a transposon donor plasmid.

FIG. 2 shows the relative transposition efficiency results of TCM_A_A2, TCM_A_B2, TCM_A_B5, TCM_A_C6, TCM_A_C9, TCM_A_D1, TCM_A_D3, TCM_A_D4, TCM_A_F8, TCM_A_G6, TCM_B_C4, TCM_B_C10, TCM_B_C11, TCM_B_C12, TCM_B_D1, TCM_B_D4, TCM_B_D5, TCM_B_D6, TCM_B_D7, TCM_B_D8, TCM_B_D10, TCM_B_E1, TCM_B_E4, TCM_B_E6, TCM_B_F2, TCM_B_F3, TCM_B_F5, TCM_B_F10, TCM_B_F11, TCM_B_G2, TCM_B_G10, TCM_B_G11, TCM_C_A12, TCM_C_B2, TCM_C_B8, TCM_C_C3, TCM_D_B11 TCM_D_G9, TCM_E_A1, TCM_E_A4, TCM_E_A5, TCM_E_A6, TCM_E_A7, TCM_E_A8, TCM_E_B6, TCM_E_B8, TCM_E_B10, TCM_E_B11, TCM_E_B12, TCM_E_C1, TCM_E_C2, TCM_E_C3, TCM_E_C5, TCM_E_C6, TCM_E_C8, TCM_E_C10, TCM_E_C11, TCM_E_C12, TCM_E_D6, TCM_E_D7, TCM_E_D12 TCM_E_E6, TCM_E_E7, TCM_E_E8, TCM_E_E10, TCM_E_E11, TCM_E_E12, TCM_E_F2, TCM_E_F3, TCM_E_F5, TCM_E_F7, TCM_E_F8, TCM_E_F11, TCM_E_G4, TCM_E_G5, TCM_E_G6, TCM_E_G7, TCM_E_G9, TCM_E_G12, SB100X and PiggyBac compared to that of inactive transposases (TCM_C_D8) in 293T cells in example 2.

FIG. 3 shows the cloning screening results of TCM_A_A2, TCM_A_B2, TCM_A_B5, TCM_A_C6, TCM_A_C9, TCM_A_D1, TCM_A_D3, TCM_A_D4, TCM_A_F8, TCM_A_G6, TCM_B_C4, TCM_B_C10, TCM_B_C11, TCM_B_C12, TCM_B_D1, TCM_B_D4, TCM_B_D5, TCM_B_D6, TCM_B_D7, TCM_B_D8, TCM_B_D10, TCM_B_E1, TCM_B_E4, TCM_B_E6, TCM_B_F2, TCM_B_F3, TCM_B_F5, TCM_B_F10, TCM_B_F11, TCM_B_G2, TCM_B_G10, TCM_B_G11 TCM_C_A12, TCM_C_B2, TCM_C_B8, TCM_C_C3, TCM_D_B11, TCM_D_G9, TCM_E_A4, TCM_E_A5, TCM_E_A6, TCM_E_A7, TCM_E_A8, TCM_E_B6, TCM_E_B8, TCM_E_B10, TCM_E_C1, TCM_E_C2, TCM_E_C3, TCM_E_C5, TCM_E_C6, TCM_E_C8, TCM_E_C10, TCM_E_C11, TCM_E_C12, TCM_E_D6, TCM_E_D7, TCM_E_D12, TCM_E_E6, TCM_E_E7, TCM_E_E8, TCM_E_E10, TCM_E_E11, TCM_E_E12, TCM_E_F3, TCM_E_F5, TCM_E_F7, TCM_E_F11, TCM_E_G4, TCM_E_G6, TCM_E_G7 and TCM_E_G9 in 293T cells in example 3. Tn+ represents co-transfection of a transposase plasmid and a donor plasmid, and Tn-represents transfection of a donor plasmid only.

FIG. 4 shows the transposition activity detection results of TCM_A_A2, TCM_A_B2, TCM_A_B5, TCM_A_C6, TCM_A_C9, TCM_A_D1, TCM_A_D3, TCM_A_D4, TCM_A_F8, TCM_A_G6, TCM_B_C4, TCM_B_C10, TCM_B_C11, TCM_B_C12, TCM_B_D1, TCM_B_D4, TCM_B_D5, TCM_B_D6, TCM_B_D7, TCM_B_D8, TCM_B_D10, TCM_B_E1, TCM_B_E4, TCM_B_E6, TCM_B_F2, TCM_B_F3, TCM_B_F5, TCM_B_F10, TCM_B_F11, TCM_B_G2, TCM_B_G10, TCM_B_G11, TCM_C_A12, TCM_C_B2, TCM_C_B8, TCM_C_C3, TCM_D_B11, TCM_D_G9, TCM_E_A4, TCM_E_A5, TCM_E_A6, TCM_E_A7, TCM_E_A8, TCM_E_B6, TCM_E_B8, TCM_E_B10, TCM_E_C1, TCM_E_C2, TCM_E_C3, TCM_E_C5, TCM_E_C6, TCM_E_C8, TCM_E_C10, TCM_E_C11, TCM_E_C12, TCM_E_D6, TCM_E_D7, TCM_E_D12, TCM_E_E6, TCM_E_E7, TCM_E_E8, TCM_E_E10, TCM_E_E11, TCM_E_E12, TCM_E_F3, TCM_E_F5, TCM_E_F7, TCM_E_F11, TCM_E_G4, TCM_E_G6, TCM_E_G7 and TCM_E_G9 in 293T cells in example 3. Tn+ represents co-transfection of a transposase plasmid and a donor plasmid, and Tn-represents transfection of a donor plasmid only.

FIG. 5 shows an evolutionary branching diagram of TCM_A_A2, TCM_A_B2, TCM_A_B5, TCM_A_C6, TCM_A_C9, TCM_A_D1, TCM_A_D3, TCM_A_D4, TCM_A_F8, TCM_A_G6, TCM_B_C4, TCM_B_C10, TCM_B_C11, TCM_B_C12, TCM_B_D1, TCM_B_D4, TCM_B_D5, TCM_B_D6, TCM_B_D7, TCM_B_D8, TCM_B_D10, TCM_B_E1, TCM_B_E4, TCM_B_E6, TCM_B_F2, TCM_B_F3, TCM_B_F5, TCM_B_F10, TCM_B_F11, TCM_B_G2, TCM_B_G10, TCM_B_G11, TCM_C_A12, TCM_C_B2, TCM_C_B8, TCM_C_C3, TCM_D_B11, TCM_D_G9, TCM_E_A1, TCM_E_A4, TCM_E_A5, TCM_E_A6, TCM_E_A7, TCM_E_A8, TCM_E_B6, TCM_E_B8, TCM_E_B10, TCM_E_B11, TCM_E_B12, TCM_E_C1, TCM_E_C2, TCM_E_C3, TCM_E_C5, TCM_E_C6, TCM_E_C8, TCM_E_C10, TCM_E_C11, TCM_E_C12, TCM_E_D6, TCM_E_D7, TCM_E_D12, TCM_E_E6, TCM_E_E7, TCM_E_E8, TCM_E_E10, TCM_E_E11, TCM_E_E12, TCM_E_F2, TCM_E_F3, TCM_E_F5, TCM_E_F7, TCM_E_F8, TCM_E_F11, TCM_E_G4, TCM_E_G5, TCM_E_G6, TCM_E_G7, TCM_E_G9, TCM_E_G12 and SB100X based on protein sequences in example 2.

FIG. 6 shows the results of protein sequence similarity among TCM_A_A2, TCM_A_B2, TCM_A_B5, TCM_A_C6, TCM_A_C9, TCM_A_D1, TCM_A_D3, TCM_A_D4, TCM_A_F8, TCM_A_G6, TCM_B_C4, TCM_B_C10, TCM_B_C11, TCM_B_C12, TCM_B_D1, TCM_B_D4, TCM_B_D5, TCM_B_D6, TCM_B_D7, TCM_B_D8, TCM_B_D10, TCM_B_E1, TCM_B_E4, TCM_B_E6, TCM_B_F2, TCM_B_F3, TCM_B_F5, TCM_B_F10, TCM_B_F11, TCM_B_G2, TCM_B_G10, TCM_B_G11, TCM_C_A12, TCM_C_B2, TCM_C_B8, TCM_C_C3, TCM_D_B11, TCM_D_G9, TCM_E_A1, TCM_E_A4, TCM_E_A5, TCM_E_A6, TCM_E_A7, TCM_E_A8, TCM_E_B6, TCM_E_B8, TCM_E_B10, TCM_E_B11, TCM_E_B12, TCM_E_C1, TCM_E_C2, TCM_E_C3, TCM_E_C5, TCM_E_C6, TCM_E_C8, TCM_E_C10, TCM_E_C11, TCM_E_C12, TCM_E_D6, TCM_E_D7, TCM_E_D12, TCM_E_E6, TCM_E_E7, TCM_E_E8, TCM_E_E10, TCM_E_E11, TCM_E_E12, TCM_E_F2, TCM_E_F3, TCM_E_F5, TCM_E_F7, TCM_E_F8, TCM_E_F11, TCM_E_G4, TCM_E_G5, TCM_E_G6, TCM_E_G7, TCM_E_G9, TCM_E_G12 and SB100X based on protein sequences in example 2.

### DETAILED DESCRIPTION OF EMBODIMENTS

Unless otherwise indicated or contradicts the context, the terms or expressions used herein should be read in conjunction with the entire content of the present disclosure and as understood by those of ordinary skill in the art. All technical and scientific terms used herein have the same meanings as commonly understood by those of ordinary skill in the art, unless otherwise defined.

In the present application, the terms “nucleic acid” and “polynucleotide” are used interchangeably, and refer to polymerization forms of nucleotides of any length, including deoxyribonucleotides, ribonucleotides, combinations thereof, and analogs thereof.

In the present application, the terms “polypeptide” and “peptide” are used interchangeably, and refer to polymers of amino acids of any length. Therefore, polypeptides, oligopeptides, proteins, antibodies and enzymes are all included in the definition of polypeptide.

As described in the present application, the “fragment” of a sequence refers to a portion of a sequence. For example, the fragment of a nucleic acid sequence refers to a portion of the nucleic acid sequence, and the fragment of an amino acid sequence refers to a portion of the amino acid sequence.

As described in the present application, a “variant” of a sequence is a polynucleotide or polypeptide that differs from a reference polynucleotide or polypeptide, respectively, but retains essential properties. A typical variant of a polynucleotide differs in nucleic acid sequence from another reference polynucleotide, and the differences in nucleic acid sequence may or may not alter the amino acid sequence of the polypeptide encoded by the reference polynucleotide. A typical variant of a polypeptide differs in amino acid sequence from another reference polypeptide. Generally, the differences are limited so that the sequences of the reference polypeptide and the variant are generally very similar, and are identical in many regions. A variant polypeptide and a reference polypeptide may differ in amino acid sequence by one or more substitutions, additions, deletions in any combination. The substituted or inserted amino acid residue may or may not be a residue encoded by the genetic code. Variants of polynucleotides or polypeptides may be naturally occurring, such as allelic variations, or they may be unknown naturally occurring variants. Non-naturally occurring polynucleotide and polypeptide variants can be produced by mutagenesis techniques, direct synthesis, and other recombinant methods known to the skilled artisan.

Amino acids are usually classified by the properties of their side chains. For example, side chains may render amino acids weak acids (e.g., amino acids D and E) or weak bases (e.g., amino acids K, R and H); and if the side chains are polar, the amino acids become hydrophilic (e.g., amino acids L and I), or if the side chains are nonpolar, the amino acids become hydrophobic (e.g., amino acids S and C).

The term “family” as used in the present application refers to a group of nucleic acids or proteins having high structural similarity produced by the same ancestor by means of replication and variation, which usually have related or even the same functions. The “superfamily” refers to a group of nucleic acids or proteins having roughly the same structure produced by the same ancestor by means of replication and variation, which belong to different families and usually have different functions.

The term “transposase” as used in the present application refers to a polypeptide that catalyzes the excision of a transposon (comprising an exogenous nucleic acid and transposase recognition sequences at both sides thereof) from a first nucleic acid (avector comprising a transposase recognition sequence and an exogenous nucleic acid) and the integration into a second nucleic acid, i.e., a target site (for example, a genomic or extrachromosomal DNA comprising a target site duplication (TSD) sequence in a cell). In some embodiments, the transposase binds to at least one terminal inverted repeat (TIR).

The term “recognition sequence” as used in the present application refer to the nucleic acid sequence located at both ends of a transposable element and one flanking a transposable first nucleic acid sequence, wherein the recognition sequence located at the 5′ end of the first nucleic acid sequence is called the 5′ recognition sequence, and the recognition sequence located at the 3′ end of the first nucleic acid sequence is called the 3′ recognition sequence. In some embodiments, the recognition sequence comprises at least one terminal inverted repeat that can bind to a transposase.

The term “nucleic acid construct” as used in the present application is defined as a single-stranded or double-stranded nucleic acid molecule herein, and preferably refers to an artificially constructed nucleic acid molecule. Optionally, the nucleic acid construct further includes one or more operably linked regulatory sequences, which can direct the expression of a coding sequence in a suitable host cell under compatible conditions. The term “expression” is understood to include any step involved in the production of a protein or polypeptide, including, but not limited to, transcription, post-transcriptional modification, translation, post-translational modification and secretion. The term “regulatory sequence” includes all components necessary or advantageous for expression of the polypeptide/protein of the present application. Each regulatory sequence may be naturally present or exogenous to the nucleic acid sequence encoding the protein or polypeptide. These regulatory sequences include, but are not limited to, leader sequences, polyadenylation sequences, propeptide sequences, promoters, signal sequences, and transcription terminators. At a minimum, the regulatory sequences should include promoters and initiation and termination signals for transcription and translation. Regulatory sequences with linkers can be provided for the purpose of introduction into specific restriction sites for linking the regulatory sequences to the coding region of a nucleic acid sequence encoding a protein or polypeptide.

The term “promoter” as used in the present application refers to a polynucleotide sequence that can control the transcription of a coding sequence. Promoter sequences include specific sequences sufficient to enable RNA polymerase to recognize, bind, and initiate transcription. In addition, promoter sequences may include sequences that optionally modulate the recognition, binding and transcription initiation activities of RNA polymerase in the nucleic acid construct or the nucleic acid set construct provided in the present application. A promoter can affect the transcription of a gene located on the same nucleic acid molecule as the promoter or a gene located on a different nucleic acid molecule from the promoter.

The term “exogenous nucleic acid fragment” used in the present application includes any gene of interest or any gene or fragment thereof that is transposable. In some non-limiting embodiments, the exogenous nucleic acid fragment is of a different origin than the terminal repeat, for example, a nucleic acid sequence isolated from an organism different from that of the terminal inverted repeat, that is, the exogenous nucleic acid fragment is exogenous to the terminal inverted repeat. In some non-limiting embodiments, the exogenous nucleic acid fragment is of a different origin than the host cell, for example, a nucleic acid sequence isolated from an organism different from the host cell, i.e., the exogenous nucleic acid fragment is exogenous to the host cell.

The term “host cell” as used in the present application include, but are not limited to, an animal cell, a plant cell, an algal cell, a fungal cell, a yeast cell, or a bacterial cell. This term includes a progeny of an original cell into which an exogenous nucleic acid fragment has been introduced. Exemplary host cell includes human embryonic kidney cell HEK293T. It is understood that, due to natural, accidental or intentional mutations, the progeny of a single parent cell may not necessarily be identical to the original parent morphologically or in terms of genome or total DNA complement.

The term “vector” as used in the present application refers to a nucleic acid molecule capable of transporting another nucleic acid molecule connected to it. Examples of vectors include, but are not limited to, plasmids, viruses, bacteria, phages, and insertable DNA fragments. The term “plasmid” refers to a circular double-stranded DNA capable of accepting an exogenous nucleic acid fragment and replicating in prokaryotic or eukaryotic cells.

### Transposase

The present application provides an isolated transposase, wherein the transposase has a transposase sequence selected from the following (i) or a variant sequence of the aforementioned transposase having a transposase activity in (ii)-(iv): (i) at least one amino acid sequence as shown in any one of SEQ ID NOs: 1-79; (ii) at least one of sequences obtained by performing deletion, substitution, insertion, or mutation of 1, 2, 3, 4, 5, 6, 7, 8, 9 or 10 amino acids on the amino acid sequence as shown in any one of SEQ ID NOs: 1-79; (iii) at least one of amino acid sequences having at least 70%, 80%, 90%, 95% or 99% identity to the amino acid sequence as shown in any one of SEQ ID NOs: 1-79; and (iv) at least one of sequences obtained by further fusing the amino acid sequence as shown in any one of SEQ ID NOs: 1-79 with other sequences.

In some embodiments, the transposase has a transposase sequence selected from at least one of the following groups (1)-(8): (1) at least one amino acid sequence as shown in any one of SEQ ID NOs: 11-36 and 59-79; (2) at least one amino acid sequence as shown in any one of SEQ ID NOs: 3-4 and 48-55; (3) at least one amino acid sequence as shown in any one of SEQ ID NOs: 5-8 and 40-44; (4) at least one amino acid sequence as shown in any one of SEQ ID NOs: 1-2 and 45-47; (5) at least one amino acid sequence as shown in any one of SEQ ID NOs: 9-10 and 56-58; (6) at least one amino acid sequence as shown in any one of SEQ ID NOs: 38-39; (7) at least one amino acid sequence as shown in any one of SEQ ID NOs: 37; and (8) an amino acid sequence having at least 70% identity to any one of SEQ ID NOs: 1-79 in the aforementioned (1)-(7).

According to an embodiment of the present application, an isolated transposase can be provided, wherein the transposase comprises an amino acid sequence as shown in formula (I)

DE (I),

wherein D is aspartic acid; and E is glutamic acid.

According to an embodiment of the present application, an isolated transposase can be provided, wherein the transposase comprises an amino acid sequence as shown in formula (II):

D(X₁)ₐH (II),

wherein D is aspartic acid; H is histidine; a is the number of amino acids; and (X₁) is any amino acid, and a is 5.

According to an embodiment of the present application, an isolated transposase can be provided, wherein the transposase comprises an amino acid sequence as shown in formula (III):

P(X₂)(X₃) (III),

wherein P is proline; X₂ is any amino acid; and X₃ is aspartic acid or glutamic acid.

According to an embodiment of the present application, an isolated transposase can be provided, wherein the transposase comprises at least two of the amino acid sequences as shown in formula (I), formula (II), and formula (III).

According to an embodiment of the present application, an isolated transposase can be provided, wherein the transposase comprises the amino acid sequences as shown in formula (I), formula (II), and formula (III).

In some embodiments, formula (I) and formula (II) are spaced by 80 to 120 amino acids, and formula (II) and formula (III) are spaced by 20 to 40 amino acids.

In some embodiments, the transposase belongs to the Tc1/mariner superfamily.

In some embodiments, the transposase belongs to the Tc1, Tc2, Tc4, Mariner, Tigger, Pogo, Fot1, ISRm11, or m44 family.

In some embodiments, the species sources of the transposase include arthropoda, chordata, Cnidaria, mollusca, or Platyhelminthes. In some embodiments, the species sources of the transposase include insecta, Actinopteri, Amphibia, Malacostraca, Arachnida, Chondrichthyes, Sauropsida, bivalvia, Ascidiacea, Hydrozoa, or Rhabditophora. In some embodiments, the species sources of the transposase include aelia acuminata, Agrypnus murinus, Albula glossodonta, Amblyraja radiata, Anthonomus grandis, Astatotilapia calliptera, Blattella germanica, Bufo gargarizans, Carassius auratus, Cephalopholis sonnerati, Cerceris rybyensis, Cheilinus undulatus, Chelonia mydas, Clitarchus hookeri, Crassostrea gigas, Cromileptes altivelis, Cyprinus carpio, Danio rerio, Drosophila ananassae, Drosophila mojavensis, Epicauta chinensis, Folsomia candida, Formica aquilonia x Formica polyctena, Gasterosteus aculeatus, Gonioctena quinquepunctata, Gymnosoma rotundatum, Harpegnathos saltator, Hemibagrus wyckioides, Homalodisca vitripennis, Hydra vulgaris, Ischnura elegans, Lampris incognitus, Laothoe populi, Latimeria chalumnae, Leistus spinibarbis, Lottia gigantea, Olea europaea subsp. europaea, Orgyia antiqua, Parhyale hawaiensis, Petrochromis sp. ‘moshi yellow’ AB-2019, Philaenus spumarius, Philereme vetulata, Phytophthora ramorum, Polistes metricus, Rana pipiens, Rhinella marina, Rhodnius prolixus, Salmo salar, Schmidtea mediterranea, Seladonia tumulorum, Sesia apiformis, Sitophilus oryzae, Solenopsis invicta, Thalassophryne amazonica, Thecocarcelia acutangulata, Thymallus thymallus, Tribolium castaneum, Vandiemenella viatica, or Zaprionus bogoriensis.

According to an embodiment of the present application, a nucleic acid can be provided, wherein, the nucleic acid encodes the transposase described in the present application.

According to an embodiment of the present application, a nucleic acid construct can be provided, comprising a nucleic acid encoding the transposase described in the present application. In some embodiments, the nucleic acid construct further comprises a promoter. The promoter can be any suitable promoter sequence, that is, a nucleic acid sequence that can be recognized by a host cell expressing the nucleic acid sequence. The promoter sequence contains a transcriptional regulatory sequence that mediates the expression of the protein or polypeptide. The promoter can be any nucleic acid sequence having transcriptional activity in a selected host cell, including mutant, truncated and heterozygous promoters, and can be derived from genes encoding extracellular or intracellular proteins or polypeptides homologous or heterologous to the host cell. In some embodiments, the promoter includes CMV, EF1a, SV40, PGK, UbC, human beta actin, CAG, TRE, UAS, Ac5, GFAP, Polyhedrin promotor, TBG, ALB, ApoEHCR-hAAT, CaMKIIa, GAL1, TEF1, GDS, ADH1, CaMV35S, Ubi, H1, U6, T7, T71ac, Sp6, araBAD, trp, lac, Ptac, or pL.

In some embodiments, the nucleic acid construct further comprises a polyadenylation [poly (A)] signal sequence. Poly (A) tailing signal sequences well known in the art, as well as various truncated forms of poly (A) tailing signals, can be used in the present application.

In some embodiments, the nucleic acid construct further includes any transcription termination sequence, i.e., a sequence that is recognized by the host cell to terminate transcription. The termination sequence is operably linked to the 3′-terminus of the nucleic acid sequence encoding the protein or polypeptide. Any terminator that is functional in the host cell of choice can be used in the present invention.

Optionally, the nucleic acid construct may further include a suitable leader sequence, that is, an untranslated region in the mRNA that is important for translation in the host cell. The leader sequence is operably linked to the 5′-terminus of the nucleic acid sequence encoding the polypeptide. Any leader sequence that is functional in the host cell of choice can be used in the present invention.

Optionally, the nucleic acid construct may further include a propeptide coding region, which encodes an amino acid sequence located at the amino terminus of the polypeptide. The resulting polypeptide is called a zymogen or propolypeptide. The propolypeptide is usually inactive and can be converted into a mature active polypeptide by catalytic or autocatalytic cleavage of the propeptide from the propolypeptide.

Optionally, the nucleic acid construct may further include a regulatory sequence that can regulate the expression of the polypeptide according to the growth conditions of the host cell. Examples of the regulatory sequence are systems that turn gene expression on or off in response to chemical or physical stimuli, including in the presence of regulatory compounds. Other examples of the regulatory sequence are those that enable gene amplification. In these instances, the nucleic acid sequence encoding the protein or polypeptide should be operably linked to the regulatory sequence.

### Nucleic Acid Construct

According to an embodiment of the present application, a nucleic acid set can be provided, comprising a 5′ recognition sequence, wherein the 5′ recognition sequence comprises at least one of the nucleotide sequences as shown in SEQ ID NOs: 80-158.

According to an embodiment of the present application, a nucleic acid set can be provided, comprising a 3′ recognition sequence, wherein the 3′ recognition sequence comprises at least one of the nucleotide sequences as shown in SEQ ID NOs: 159-237.

According to an embodiment of the present application, a nucleic acid set can be provided, comprising a 5′ recognition sequence and a 3′ recognition sequence, wherein the 5′ recognition sequence comprises the nucleotide sequence as shown in any one of SEQ ID NOs: 80-158 or a variant thereof, the 3′ recognition sequence comprises the nucleotide sequence as shown in any one of SEQ ID NOs: 159-237 or a variant thereof, and the nucleic acid set can be recognized by a specific transposase.

In some embodiments, the 5′ recognition sequence or the 3′ recognition sequence comprises a terminal inverted repeat of at least one of 1-1200 nt, 1-800 nt, 1-600 nt, 1-400 nt, 1-200 nt, 1-100 nt, 5-80 nt, 10-70 nt, or 20-60 nt in length.

In some embodiments, the 5′ recognition sequence or the 3′ recognition sequence comprises a terminal inverted repeat of at least one of 1-800 nt, 20-700 nt, 50-600 nt, 100-500 nt, 150-400 nt, 150-300 nt, or 200-260 nt in length.

According to an embodiment of the present application, a nucleic acid set construct can be provided, the nucleic acid set construct includes the nucleic acid set described in the present application and further includes an exogenous nucleic acid fragment. In some embodiments, the exogenous nucleic acid fragment is operably inserted into the nucleic acid set construct through a polyclonal insertion site, and there may be one or more exogenous nucleic acid fragments, which may be the same or different; and a promoter can also be inserted to control the expression of the exogenous nucleic acid fragment. In some embodiments, the exogenous nucleic acid fragment includes any gene of interest or any gene that is transposable, e.g., a gene of a natural functional protein, an artificial chimeric gene, or a gene of a non-coding RNA (ncRNA). In some embodiments, the gene of a non-coding RNA includes a variety of RNAs with known functions and RNAs with unknown functions, such as rRNA, tRNA, small interfering RNA (siRNA), small nuclear RNA (snRNA), small nucleolar RNA (snoRNA), and microRNA (miRNA). In some embodiments, the gene of a natural functional protein includes a fluorescence-based reporter gene, a luciferase gene, and a resistance gene. In some embodiments, the artificial chimeric gene includes a gene of a chimeric antigen receptor. In some embodiments, the fluorescence-based reporter gene is selected from at least one of genes encoding a green fluorescent protein, a red fluorescent protein, a blue fluorescent protein, or a yellow fluorescent protein. In some embodiments, the luciferase gene is selected from at least one of genes encoding firefly luciferase and sea kidney luciferase. In some embodiments, the resistance gene is selected from at least one of genes encoding puromycin resistance, G418 resistance, kanamycin resistance, tetracycline resistance, and bleomycin resistance.

In some embodiments, a promoter can also be inserted into the nucleic acid set construct to control the expression of the exogenous nucleic acid fragment. The promoter can be any suitable promoter sequence, that is, a nucleic acid sequence that can be recognized by a host cell expressing the exogenous nucleic acid fragment. The promoter sequence contains a transcriptional regulatory sequence that mediates the expression of the protein or polypeptide. The promoter can be any nucleic acid sequence having transcriptional activity in a selected host cell, including mutant, truncated and heterozygous promoters, and can be derived from genes encoding extracellular or intracellular proteins or polypeptides homologous or heterologous to the host cell. In some embodiments, the promoter includes CMV, EF1a, SV40, PGK, UbC, human beta actin, CAG, TRE, UAS, Ac5, GFAP, Polyhedrin promotor, TBG, ALB, ApoEHCR-hAAT, CaMKIIa, GAL1, TEF1, GDS, ADH1, CaMV35S, Ubi, H1, U6, T7, T71ac, Sp6, araBAD, trp, lac, Ptac, or pL.

In some embodiments, the nucleic acid set construct further includes any transcription termination sequence (i.e., a sequence that is recognized by the host cell to terminate transcription) to control the expression of the exogenous nucleic acid fragment. Any terminator that is functional in the host cell of choice can be used in the present invention.

Optionally, the nucleic acid set construct may further include a suitable leader sequence (i.e., an untranslated region in the mRNA that is important for translation in the host cell) to control the expression of the exogenous nucleic acid fragment. The leader sequence is operably linked to the 5′-terminus of the nucleic acid sequence encoding the polypeptide. Any leader sequence that is functional in the host cell of choice can be used in the present invention.

Optionally, the nucleic acid set construct may further include a propeptide coding region to control the expression of the exogenous nucleic acid fragment, the propeptide coding region encodes an amino acid sequence located at the amino terminus of the polypeptide. The resulting polypeptide is called a zymogen or propolypeptide. The propolypeptide is usually inactive and can be converted into a mature active polypeptide by catalytic or autocatalytic cleavage of the propeptide from the propolypeptide.

Optionally, the nucleic acid set construct may further include a regulatory sequence that can regulate the expression of the exogenous nucleic acid fragment according to the growth conditions of the host cell. Examples of the regulatory sequence are systems that turn gene expression on or off in response to chemical or physical stimuli, including in the presence of regulatory compounds. Other examples of the regulatory sequence are those that enable gene amplification. In these instances, the exogenous nucleic acid fragment should be operably linked to the regulatory sequence.

### Transposition Composition

According to an embodiment of the present application, a composition may be provided, wherein, the composition includes: a Tc1/mariner superfamily transposase or a functional fragment thereof, or a nucleic acid encoding the Tc1/mariner superfamily transposase or the functional fragment thereof, wherein the transposase or the functional fragment thereof has a function of catalyzing the insertion of an exogenous nucleic acid fragment into the genome of a cell; and a nucleic acid set, wherein the nucleic acid set can be recognized by a specific transposase or a functional fragment thereof.

In some embodiments, the composition is selected from at least one of the following groups (1)-(80), and any one of the following groups (1)-(79) comprises: a transposase-related sequence and a nucleic acid set,
(1) the transposase-related sequence is an amino acid sequence comprising the sequence as shown in SEQ ID NO: 1 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 80, and the 3′ recognition sequence is a nucleotide sequence comprising the sequence as shown in SEQ ID NO: 159;
(2) the transposase-related sequence is an amino acid sequence comprising the sequence as shown in SEQ ID NO: 2 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 81, and the 3′ recognition sequence is a nucleotide sequence comprising the sequence as shown in SEQ ID NO: 160;
(3) the transposase-related sequence is an amino acid sequence comprising the sequence as shown in SEQ ID NO: 3 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 82, and the 3′ recognition sequence is a nucleotide sequence comprising the sequence as shown in SEQ ID NO: 161;
(4) the transposase-related sequence is an amino acid sequence comprising the sequence as shown in SEQ ID NO: 4 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 83, and the 3′ recognition sequence is a nucleotide sequence comprising the sequence as shown in SEQ ID NO: 162;
(5) the transposase-related sequence is an amino acid sequence comprising the sequence as shown in SEQ ID NO: 5 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 84, and the 3′ recognition sequence is a nucleotide sequence comprising the sequence as shown in SEQ ID NO: 163;
(6) the transposase-related sequence is an amino acid sequence comprising the sequence as shown in SEQ ID NO: 6 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 85, and the 3′ recognition sequence is a nucleotide sequence comprising the sequence as shown in SEQ ID NO: 164;
(7) the transposase-related sequence is an amino acid sequence comprising the sequence as shown in SEQ ID NO: 7 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 86, and the 3′ recognition sequence is a nucleotide sequence comprising the sequence as shown in SEQ ID NO: 165;
(8) the transposase-related sequence is an amino acid sequence comprising the sequence as shown in SEQ ID NO: 8 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 87, and the 3′ recognition sequence is a nucleotide sequence comprising the sequence as shown in SEQ ID NO: 166;
(9) the transposase-related sequence is an amino acid sequence comprising the sequence as shown in SEQ ID NO: 9 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 88, and the 3′ recognition sequence is a nucleotide sequence comprising the sequence as shown in SEQ ID NO: 167;
(10) the transposase-related sequence is an amino acid sequence comprising the sequence as shown in SEQ ID NO: 10 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 89, and the 3′ recognition sequence is a nucleotide sequence comprising the sequence as shown in SEQ ID NO: 168;
(11) the transposase-related sequence is an amino acid sequence comprising the sequence as shown in SEQ ID NO: 11 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 90, and the 3′ recognition sequence is a nucleotide sequence comprising the sequence as shown in SEQ ID NO: 169;
(12) the transposase-related sequence is an amino acid sequence comprising the sequence as shown in SEQ ID NO: 12 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 91, and the 3′ recognition sequence is a nucleotide sequence comprising the sequence as shown in SEQ ID NO: 170;
(13) the transposase-related sequence is an amino acid sequence comprising the sequence as shown in SEQ ID NO: 13 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 92, and the 3′ recognition sequence is a nucleotide sequence comprising the sequence as shown in SEQ ID NO: 171;
(14) the transposase-related sequence is an amino acid sequence comprising the sequence as shown in SEQ ID NO: 14 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 93, and the 3′ recognition sequence is a nucleotide sequence comprising the sequence as shown in SEQ ID NO: 172;
(15) the transposase-related sequence is an amino acid sequence comprising the sequence as shown in SEQ ID NO: 15 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 94, and the 3′ recognition sequence is a nucleotide sequence comprising the sequence as shown in SEQ ID NO: 173;
(16) the transposase-related sequence is an amino acid sequence comprising the sequence as shown in SEQ ID NO: 16 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 95, and the 3′ recognition sequence is a nucleotide sequence comprising the sequence as shown in SEQ ID NO: 174;
(17) the transposase-related sequence is an amino acid sequence comprising the sequence as shown in SEQ ID NO: 17 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 96, and the 3′ recognition sequence is a nucleotide sequence comprising the sequence as shown in SEQ ID NO: 175;
(18) the transposase-related sequence is an amino acid sequence comprising the sequence as shown in SEQ ID NO: 18 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 97, and the 3′ recognition sequence is a nucleotide sequence comprising the sequence as shown in SEQ ID NO: 176;
(19) the transposase-related sequence is an amino acid sequence comprising the sequence as shown in SEQ ID NO: 19 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 98, and the 3′ recognition sequence is a nucleotide sequence comprising the sequence as shown in SEQ ID NO: 177;
(20) the transposase-related sequence is an amino acid sequence comprising the sequence as shown in SEQ ID NO: 20 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 99, and the 3′ recognition sequence is a nucleotide sequence comprising the sequence as shown in SEQ ID NO: 178;
(21) the transposase-related sequence is an amino acid sequence comprising the sequence as shown in SEQ ID NO: 21 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 100, and the 3′ recognition sequence is a nucleotide sequence comprising the sequence as shown in SEQ ID NO: 179;
(22) the transposase-related sequence is an amino acid sequence comprising the sequence as shown in SEQ ID NO: 22 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 101, and the 3′ recognition sequence is a nucleotide sequence comprising the sequence as shown in SEQ ID NO: 180;
(23) the transposase-related sequence is an amino acid sequence comprising the sequence as shown in SEQ ID NO: 23 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 102, and the 3′ recognition sequence is a nucleotide sequence comprising the sequence as shown in SEQ ID NO: 181;
(24) the transposase-related sequence is an amino acid sequence comprising the sequence as shown in SEQ ID NO: 24 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 103, and the 3′ recognition sequence is a nucleotide sequence comprising the sequence as shown in SEQ ID NO: 182;
(25) the transposase-related sequence is an amino acid sequence comprising the sequence as shown in SEQ ID NO: 25 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 104, and the 3′ recognition sequence is a nucleotide sequence comprising the sequence as shown in SEQ ID NO: 183;
(26) the transposase-related sequence is an amino acid sequence comprising the sequence as shown in SEQ ID NO: 26 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 105, and the 3′ recognition sequence is a nucleotide sequence comprising the sequence as shown in SEQ ID NO: 184;
(27) the transposase-related sequence is an amino acid sequence comprising the sequence as shown in SEQ ID NO: 27 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 106, and the 3′ recognition sequence is a nucleotide sequence comprising the sequence as shown in SEQ ID NO: 185;
(28) the transposase-related sequence is an amino acid sequence comprising the sequence as shown in SEQ ID NO: 28 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 107, and the 3′ recognition sequence is a nucleotide sequence comprising the sequence as shown in SEQ ID NO: 186;
(29) the transposase-related sequence is an amino acid sequence comprising the sequence as shown in SEQ ID NO: 29 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 108, and the 3′ recognition sequence is a nucleotide sequence comprising the sequence as shown in SEQ ID NO: 187;
(30) the transposase-related sequence is an amino acid sequence comprising the sequence as shown in SEQ ID NO: 30 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 109, and the 3′ recognition sequence is a nucleotide sequence comprising the sequence as shown in SEQ ID NO: 188;
(31) the transposase-related sequence is an amino acid sequence comprising the sequence as shown in SEQ ID NO: 31 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 110, and the 3′ recognition sequence is a nucleotide sequence comprising the sequence as shown in SEQ ID NO: 189;
(32) the transposase-related sequence is an amino acid sequence comprising the sequence as shown in SEQ ID NO: 32 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 111, and the 3′ recognition sequence is a nucleotide sequence comprising the sequence as shown in SEQ ID NO: 190;
(33) the transposase-related sequence is an amino acid sequence comprising the sequence as shown in SEQ ID NO: 33 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 112, and the 3′ recognition sequence is a nucleotide sequence comprising the sequence as shown in SEQ ID NO: 191;
(34) the transposase-related sequence is an amino acid sequence comprising the sequence as shown in SEQ ID NO: 34 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 113, and the 3′ recognition sequence is a nucleotide sequence comprising the sequence as shown in SEQ ID NO: 192;
(35) the transposase-related sequence is an amino acid sequence comprising the sequence as shown in SEQ ID NO: 35 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 114, and the 3′ recognition sequence is a nucleotide sequence comprising the sequence as shown in SEQ ID NO: 193;
(36) the transposase-related sequence is an amino acid sequence comprising the sequence as shown in SEQ ID NO: 36 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 115, and the 3′ recognition sequence is a nucleotide sequence comprising the sequence as shown in SEQ ID NO: 194;
(37) the transposase-related sequence is an amino acid sequence comprising the sequence as shown in SEQ ID NO: 37 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 116, and the 3′ recognition sequence is a nucleotide sequence comprising the sequence as shown in SEQ ID NO: 195;
(38) the transposase-related sequence is an amino acid sequence comprising the sequence as shown in SEQ ID NO: 38 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 117, and the 3′ recognition sequence is a nucleotide sequence comprising the sequence as shown in SEQ ID NO: 196;
(39) the transposase-related sequence is an amino acid sequence comprising the sequence as shown in SEQ ID NO: 39 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 118, and the 3′ recognition sequence is a nucleotide sequence comprising the sequence as shown in SEQ ID NO: 197;
(40) the transposase-related sequence is an amino acid sequence comprising the sequence as shown in SEQ ID NO: 40 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 119, and the 3′ recognition sequence is a nucleotide sequence comprising the sequence as shown in SEQ ID NO: 198;
(41) the transposase-related sequence is an amino acid sequence comprising the sequence as shown in SEQ ID NO: 41 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 120, and the 3′ recognition sequence is a nucleotide sequence comprising the sequence as shown in SEQ ID NO: 199;
(42) the transposase-related sequence is an amino acid sequence comprising the sequence as shown in SEQ ID NO: 42 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 121, and the 3′ recognition sequence is a nucleotide sequence comprising the sequence as shown in SEQ ID NO: 200;
(43) the transposase-related sequence is an amino acid sequence comprising the sequence as shown in SEQ ID NO: 43 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 122, and the 3′ recognition sequence is a nucleotide sequence comprising the sequence as shown in SEQ ID NO: 201;
(44) the transposase-related sequence is an amino acid sequence comprising the sequence as shown in SEQ ID NO: 44 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 123, and the 3′ recognition sequence is a nucleotide sequence comprising the sequence as shown in SEQ ID NO: 202;
(45) the transposase-related sequence is an amino acid sequence comprising the sequence as shown in SEQ ID NO: 45 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 124, and the 3′ recognition sequence is a nucleotide sequence comprising the sequence as shown in SEQ ID NO: 203;
(46) the transposase-related sequence is an amino acid sequence comprising the sequence as shown in SEQ ID NO: 46 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 125, and the 3′ recognition sequence is a nucleotide sequence comprising the sequence as shown in SEQ ID NO: 204;
(47) the transposase-related sequence is an amino acid sequence comprising the sequence as shown in SEQ ID NO: 47 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 126, and the 3′ recognition sequence is a nucleotide sequence comprising the sequence as shown in SEQ ID NO: 205;
(48) the transposase-related sequence is an amino acid sequence comprising the sequence as shown in SEQ ID NO: 48 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 127, and the 3′ recognition sequence is a nucleotide sequence comprising the sequence as shown in SEQ ID NO: 206;
(49) the transposase-related sequence is an amino acid sequence comprising the sequence as shown in SEQ ID NO: 49 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 128, and the 3′ recognition sequence is a nucleotide sequence comprising the sequence as shown in SEQ ID NO: 207;
(50) the transposase-related sequence is an amino acid sequence comprising the sequence as shown in SEQ ID NO: 50 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 129, and the 3′ recognition sequence is a nucleotide sequence comprising the sequence as shown in SEQ ID NO: 208;
(51) the transposase-related sequence is an amino acid sequence comprising the sequence as shown in SEQ ID NO: 51 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 130, and the 3′ recognition sequence is a nucleotide sequence comprising the sequence as shown in SEQ ID NO: 209;
(52) the transposase-related sequence is an amino acid sequence comprising the sequence as shown in SEQ ID NO: 52 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 131, and the 3′ recognition sequence is a nucleotide sequence comprising the sequence as shown in SEQ ID NO: 210;
(53) the transposase-related sequence is an amino acid sequence comprising the sequence as shown in SEQ ID NO: 53 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 132, and the 3′ recognition sequence is a nucleotide sequence comprising the sequence as shown in SEQ ID NO: 211;
(54) the transposase-related sequence is an amino acid sequence comprising the sequence as shown in SEQ ID NO: 54 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 133, and the 3′ recognition sequence is a nucleotide sequence comprising the sequence as shown in SEQ ID NO: 212;
(55) the transposase-related sequence is an amino acid sequence comprising the sequence as shown in SEQ ID NO: 55 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 134, and the 3′ recognition sequence is a nucleotide sequence comprising the sequence as shown in SEQ ID NO: 213;
(56) the transposase-related sequence is an amino acid sequence comprising the sequence as shown in SEQ ID NO: 56 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 135, and the 3′ recognition sequence is a nucleotide sequence comprising the sequence as shown in SEQ ID NO: 214;
(57) the transposase-related sequence is an amino acid sequence comprising the sequence as shown in SEQ ID NO: 57 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 136, and the 3′ recognition sequence is a nucleotide sequence comprising the sequence as shown in SEQ ID NO: 215;
(58) the transposase-related sequence is an amino acid sequence comprising the sequence as shown in SEQ ID NO: 58 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 137, and the 3′ recognition sequence is a nucleotide sequence comprising the sequence as shown in SEQ ID NO: 216;
(59) the transposase-related sequence is an amino acid sequence comprising the sequence as shown in SEQ ID NO: 59 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 138, and the 3′ recognition sequence is a nucleotide sequence comprising the sequence as shown in SEQ ID NO: 217;
(60) the transposase-related sequence is an amino acid sequence comprising the sequence as shown in SEQ ID NO: 60 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 139, and the 3′ recognition sequence is a nucleotide sequence comprising the sequence as shown in SEQ ID NO: 218;
(61) the transposase-related sequence is an amino acid sequence comprising the sequence as shown in SEQ ID NO: 61 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 140, and the 3′ recognition sequence is a nucleotide sequence comprising the sequence as shown in SEQ ID NO: 219;
(62) the transposase-related sequence is an amino acid sequence comprising the sequence as shown in SEQ ID NO: 62 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 141, and the 3′ recognition sequence is a nucleotide sequence comprising the sequence as shown in SEQ ID NO: 220;
(63) the transposase-related sequence is an amino acid sequence comprising the sequence as shown in SEQ ID NO: 63 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 142, and the 3′ recognition sequence is a nucleotide sequence comprising the sequence as shown in SEQ ID NO: 221;
(64) the transposase-related sequence is an amino acid sequence comprising the sequence as shown in SEQ ID NO: 64 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 143, and the 3′ recognition sequence is a nucleotide sequence comprising the sequence as shown in SEQ ID NO: 222;
(65) the transposase-related sequence is an amino acid sequence comprising the sequence as shown in SEQ ID NO: 65 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 144, and the 3′ recognition sequence is a nucleotide sequence comprising the sequence as shown in SEQ ID NO: 223;
(66) the transposase-related sequence is an amino acid sequence comprising the sequence as shown in SEQ ID NO: 66 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 145, and the 3′ recognition sequence is a nucleotide sequence comprising the sequence as shown in SEQ ID NO: 224;
(67) the transposase-related sequence is an amino acid sequence comprising the sequence as shown in SEQ ID NO: 67 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 146, and the 3′ recognition sequence is a nucleotide sequence comprising the sequence as shown in SEQ ID NO: 225;
(68) the transposase-related sequence is an amino acid sequence comprising the sequence as shown in SEQ ID NO: 68 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 147, and the 3′ recognition sequence is a nucleotide sequence comprising the sequence as shown in SEQ ID NO: 226;
(69) the transposase-related sequence is an amino acid sequence comprising the sequence as shown in SEQ ID NO: 69 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 148, and the 3′ recognition sequence is a nucleotide sequence comprising the sequence as shown in SEQ ID NO: 227;
(70) the transposase-related sequence is an amino acid sequence comprising the sequence as shown in SEQ ID NO: 70 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 149, and the 3′ recognition sequence is a nucleotide sequence comprising the sequence as shown in SEQ ID NO: 228;
(71) the transposase-related sequence is an amino acid sequence comprising the sequence as shown in SEQ ID NO: 71 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 150, and the 3′ recognition sequence is a nucleotide sequence comprising the sequence as shown in SEQ ID NO: 229;
(72) the transposase-related sequence is an amino acid sequence comprising the sequence as shown in SEQ ID NO: 72 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 151, and the 3′ recognition sequence is a nucleotide sequence comprising the sequence as shown in SEQ ID NO: 230;
(73) the transposase-related sequence is an amino acid sequence comprising the sequence as shown in SEQ ID NO: 73 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 152, and the 3′ recognition sequence is a nucleotide sequence comprising the sequence as shown in SEQ ID NO: 231;
(74) the transposase-related sequence is an amino acid sequence comprising the sequence as shown in SEQ ID NO: 74 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 153, and the 3′ recognition sequence is a nucleotide sequence comprising the sequence as shown in SEQ ID NO: 232;
(75) the transposase-related sequence is an amino acid sequence comprising the sequence as shown in SEQ ID NO: 75 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 154, and the 3′ recognition sequence is a nucleotide sequence comprising the sequence as shown in SEQ ID NO: 233;
(76) the transposase-related sequence is an amino acid sequence comprising the sequence as shown in SEQ ID NO: 76 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 155, and the 3′ recognition sequence is a nucleotide sequence comprising the sequence as shown in SEQ ID NO: 234;
(77) the transposase-related sequence is an amino acid sequence comprising the sequence as shown in SEQ ID NO: 77 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 156, and the 3′ recognition sequence is a nucleotide sequence comprising the sequence as shown in SEQ ID NO: 235;
(78) the transposase-related sequence is an amino acid sequence comprising the sequence as shown in SEQ ID NO: 78 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 157, and the 3′ recognition sequence is a nucleotide sequence comprising the sequence as shown in SEQ ID NO: 236;
(79) the transposase-related sequence is an amino acid sequence comprising the sequence as shown in SEQ ID NO: 79 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 158, and the 3′ recognition sequence is a nucleotide sequence comprising the sequence as shown in SEQ ID NO: 237; or
(80) a variant of any one of the aforementioned groups (1)-(79),

wherein the transposase-related sequence is the amino acid sequence of the variant of the transposase in each group or a nucleic acid sequence encoding the variant, and the variant has a variant sequence of the aforementioned transposase having a transposase activity selected from the following (i)-(iii):
(i) at least one of sequences obtained by performing deletion, substitution, insertion, or mutation of 1, 2, 3, 4, 5, 6, 7, 8, 9 or 10 amino acids on the amino acid sequence of the transposase in each group;
(ii) at least one of amino acid sequences having at least 70%, 80%, 90%, 95% or 99% identity to the amino acid sequence as shown in any one of SEQ ID NOs: 1-79; and
(iii) at least one of sequences obtained by further fusing the amino acid sequence as shown in any one of SEQ ID NOs: 1-79 with other sequences.

In some embodiments, the nucleic acid encoding the amino acid sequence further comprises a promoter. The promoter can be any suitable promoter sequence, that is, a nucleic acid sequence that can be recognized by a host cell expressing the nucleic acid sequence. The promoter sequence contains a transcriptional regulatory sequence that mediates the expression of the protein or polypeptide. The promoter can be any nucleic acid sequence having transcriptional activity in a selected host cell, including mutant, truncated and heterozygous promoters, and can be derived from genes encoding extracellular or intracellular proteins or polypeptides homologous or heterologous to the host cell. In some embodiments, the promoter includes CMV, EF1a, SV40, PGK, UbC, human beta actin, CAG, TRE, UAS, Ac5, GFAP, Polyhedrin promotor, TBG, ALB, ApoEHCR-hAAT, CaMKIIa, GAL1, TEF1, GDS, ADH1, CaMV35S, Ubi, H1, U6, T7, T71ac, Sp6, araBAD, trp, lac, Ptac, or pL. In some embodiments, the nucleic acid encoding the amino acid sequence further comprises a poly (A) sequence. Poly (A) tailing signal sequences well known in the art, as well as various truncated forms of poly (A) tailing signals, can be used in the present application.

In some embodiments, the nucleic acid set further includes an exogenous nucleic acid fragment. In some embodiments, the exogenous nucleic acid fragment is operably inserted into the nucleic acid construct through a polyclonal insertion site, and there may be one or more exogenous nucleic acid fragments, which may be the same or different; and a promoter can also be inserted to control the expression of the exogenous nucleic acid fragment. In some embodiments, the exogenous nucleic acid fragment includes any gene of interest or any gene that is transposable, e.g., a gene of a natural functional protein, an artificial chimeric gene, or a gene of a non-coding RNA (ncRNA). In some embodiments, the gene of a non-coding RNA includes a variety of RNAs with known functions and RNAs with unknown functions, such as rRNA, tRNA, small interfering RNA (siRNA), small nuclear RNA (snRNA), small nucleolar RNA (snoRNA), and microRNA (miRNA). In some embodiments, the gene of a natural functional protein includes a fluorescence-based reporter gene, a luciferase gene, and a resistance gene. In some embodiments, the artificial chimeric gene includes a gene of a chimeric antigen receptor. In some embodiments, the fluorescence-based reporter gene includes a gene encoding a green fluorescent protein, a red fluorescent protein, a blue fluorescent protein, or a yellow fluorescent protein. In some embodiments, the luciferase gene includes a gene encoding firefly luciferase or sea kidney luciferase. In some embodiments, the resistance gene includes a gene encoding puromycin resistance, G418 resistance, kanamycin resistance, tetracycline resistance, or bleomycin resistance. In some embodiments, a promoter can also be inserted into the nucleic acid set to control the expression of the exogenous nucleic acid fragment. The promoter can be any suitable promoter sequence, that is, a nucleic acid sequence that can be recognized by a host cell expressing the exogenous nucleic acid fragment. The promoter sequence contains a transcriptional regulatory sequence that mediates the expression of the protein or polypeptide. The promoter can be any nucleic acid sequence having transcriptional activity in a selected host cell, including mutant, truncated and heterozygous promoters, and can be derived from genes encoding extracellular or intracellular proteins or polypeptides homologous or heterologous to the host cell. In some embodiments, the promoter includes CMV, EF1a, SV40, PGK, UbC, human beta actin, CAG, TRE, UAS, Ac5, GFAP, Polyhedrin promotor, TBG, ALB, ApoEHCR-hAAT, CaMKIIa, GAL1, TEF1, GDS, ADH1, CaMV35S, Ubi, H1, U6, T7, T71ac, Sp6, araBAD, trp, lac, Ptac, or pL.

In some embodiments, the nucleic acid encoding the amino acid sequence and/or the nucleic acid set further comprises any transcription termination sequence that controls the expression of the exogenous nucleic acid fragment, i.e., a sequence that is recognized by a host cell to terminate transcription. Any terminator that is functional in the host cell of choice can be used in the present invention.

In some embodiments, the nucleic acid encoding the amino acid sequence and/or the nucleic acid set further includes any transcription termination sequence, i.e., a sequence that is recognized by the host cell to terminate transcription. The termination sequence is operably linked to the 3′-terminus of the nucleic acid sequence encoding the protein or polypeptide. Any terminator that is functional in the host cell of choice can be used in the present invention.

Optionally, the nucleic acid encoding the amino acid sequence and/or the nucleic acid set may further comprise a suitable leader sequence, i.e., an untranslated region in the mRNA that is important for translation in the host cell. The leader sequence is operably linked to the 5′-terminus of the nucleic acid sequence encoding the polypeptide. Any leader sequence that is functional in the host cell of choice can be used in the present invention.

Optionally, the nucleic acid encoding the amino acid sequence and/or the nucleic acid set may further comprise a propeptide coding region, which encodes an amino acid sequence located at the amino terminus of the polypeptide. The resulting polypeptide is called a zymogen or propolypeptide. The propolypeptide is usually inactive and can be converted into a mature active polypeptide by catalytic or autocatalytic cleavage of the propeptide from the propolypeptide.

Optionally, the nucleic acid encoding the amino acid sequence and/or the nucleic acid set may further comprise a regulatory sequence that can regulate the expression of the polypeptide according to the growth conditions of the host cell. Examples of the regulatory sequence are systems that turn gene expression on or off in response to chemical or physical stimuli, including in the presence of regulatory compounds. Other examples of the regulatory sequence are those that enable gene amplification. In these instances, the nucleic acid sequence encoding the protein or polypeptide should be operably linked to the regulatory sequence.

### Recombinant Vector, Recombinant Host Cell and Kit

According to an embodiment of the present application, a recombinant vector can be provided, wherein, the recombinant vector comprises the nucleic acid encoding the transposase described in the present application, the nucleic acid described in the present application, the nucleic acid construct described in the present application, the nucleic acid set described in the present application, the nucleic acid set construct described in the present application, or the composition described in the present application. The recombinant vector can be any suitable vector. In some embodiments, the recombinant vector includes, but is not limited to, a recombinant cloning vector, a recombinant eukaryotic expression plasmid, or a recombinant viral vector. In some embodiments, the recombinant eukaryotic expression plasmid includes pcDNA3.1, pCMV, pUC18, pUC19, pUC57, pBAD, pET, pENTR, pGenlenti, or pAAV. In some embodiments, the recombinant virus vector includes a recombinant adenovirus vector, a recombinant adeno-associated virus vector, a recombinant retrovirus vector, a recombinant herpes simplex virus vector, or a recombinant vaccinia virus vector. The recombinant vector of the present invention can be constructed using methods well known in the art. For example, depending on the restriction sites contained in the backbone vector used, appropriate restriction sites can be added to both ends of the nucleic acid construct of the present invention, and then loaded into the backbone vector.

According to an embodiment of the present application, a recombinant host cell can be provided, wherein, the recombinant host cell comprises the transposase described in the present application, the nucleic acid encoding the transposase described in the present application, the nucleic acid described in the present application, the nucleic acid construct described in the present application, the nucleic acid set described in the present application, the nucleic acid set construct described in the present application, the composition described in the present application, or the recombinant vector described in the present application. The recombinant host cell can be any host cell in which transposases can be used. In some embodiments, the recombinant host cell includes, but is not limited to, an animal cell, a plant cell, an algal cell, a fungal cell, a yeast cell, or a bacterial cell. In some embodiments, the animal cell includes a mammalian cell. In some embodiments, the mammalian cell includes a primary cell (e.g., a mesenchymal stem cell, an endothelial cell, an epithelial cell, a fibroblast, a keratinocyte, a melanocyte, a smooth muscle cell, and an immune cell), an immortalized cell line (e.g., HEK293, NIH-3T3, RAW-264.7, STO, VERO, CT26, hTERT immortalized human endothelial/epithelial/fibroblast/keratinocyte/ductal/cell lines), a cancer cell line (e.g., Hela, HepG2/3, HL-60, HT-1080, HT-29, A549, SW620, HCT-15, HCT116, MDA-MB-231, MCF7, SK-OV-3, PANC-1, AsPc-1, THP-1, Huh7, KG-1, RAJI, HB-CB, Jurkat, K562, CRL5826, CHO, MDCK, and Renca), an embryonic stem cell line (e.g., H1, H9, WIBR2, WIBR3, G-Olig2, ESF158, RW. 4, R1, and D3) and differentiated cells thereof, or an induced pluripotent stem cell line and differentiated cells thereof.

According to an embodiment of the present application, a kit can be provided, wherein, the kit comprises the transposase described in the present application, the nucleic acid encoding the transposase described in the present application, the nucleic acid described in the present application, the nucleic acid construct described in the present application, the nucleic acid set described in the present application, the nucleic acid set construct described in the present application, the composition described in the present application, the recombinant vector described in the present application, or the recombinant host cell described in the present application.

### Method and Use

The transposase-based tools and methods for large fragment gene insertion and integration provided in the present application can be applied to many fields such as gene and cell therapy, molecular breeding in animals and plants, and industrial microorganism engineering. Particularly in the field of cell therapy, the transposition system provided by the present application can be applied to the integration of CAR sequences in cell immunotherapy (CAR-T, CAR-NK, CAR-M, etc.); in the field of gene therapy, the transposition system provided by the present application can be used to insert or integrate a healthy gene into the genome of a cell, thereby facilitating the treatment of diseases caused by gene mutations or gene defects; in terms of molecular breeding, the transposition system provided by the present application can be used as a tool for breeding many crops such as rice, corn and wheat, and can also accelerate the breeding process of animals and plants in a targeted manner; and in terms of industrial microorganism engineering, due to the defects such as instability and easy loss of plasmids in gene expression, the transposition system provided by the present application can stably integrate a gene into the chromosome of a microorganism.

According to an embodiment of the present application, a method for introducing an exogenous nucleic acid fragment into the genome of a host cell can be provided, wherein, the method comprises: delivering the transposase described in the present application, the nucleic acid encoding the transposase described in the present application, the nucleic acid described in the present application, the nucleic acid construct described in the present application, the nucleic acid set described in the present application, the nucleic acid set construct described in the present application, the composition described in the present application, or the recombinant vector described in the present application into a host cell.

According to an embodiment of the present application, a method for editing the genome of a host cell can be provided, wherein, the method comprises: delivering the transposase described in the present application, the nucleic acid encoding the transposase described in the present application, the nucleic acid described in the present application, the nucleic acid construct described in the present application, the nucleic acid set described in the present application, the nucleic acid set construct described in the present application, the composition described in the present application, or the recombinant vector described in the present application into a host cell.

According to an embodiment of the present application, a method for obtaining a host cell containing an exogenous nucleic acid fragment in the genome can be provided, wherein, the method comprises: delivering the transposase described in the present application, the nucleic acid encoding the transposase described in the present application, the nucleic acid described in the present application, the nucleic acid construct described in the present application, the nucleic acid set described in the present application, the nucleic acid set construct described in the present application, the composition described in the present application, or the recombinant vector described in the present application into a host cell.

The method of delivery into the host cell can be any suitable method. In some embodiments, the delivery method includes but is not limited to cationic liposome delivery, lipoid nanoparticulate delivery, cationic polymer delivery, vesicle-exosome delivery, gold nanoparticulate delivery, polypeptide and protein delivery, retrovirus delivery, lentivirus delivery, adenovirus delivery, adeno-associated virus delivery, electroporation, agrobacterium infection, or gene gun. The methods of cell transfection and culture are routine methods in the art, and appropriate transfection and culture methods can be selected according to different cell types.

The host cell can be any host cell in which transposases can be used. In some embodiments, the host cell includes, but is not limited to, an animal cell, a plant cell, an algal cell, a fungal cell, a yeast cell, or a bacterial cell. In some embodiments, the host cell includes a mammalian cell. In some embodiments, the host cell includes a primary cell (e.g., a mesenchymal stem cell, an endothelial cell, an epithelial cell, a fibroblast, a keratinocyte, a melanocyte, a smooth muscle cell, and an immune cell), an immortalized cell line (e.g., HEK293, NIH-3T3, RAW-264.7, STO, VERO, CT26, hTERT immortalized human endothelial/epithelial/fibroblast/keratinocyte/ductal/cell lines), a cancer cell line (e.g., Hela, HepG2/3, HL-60, HT-1080, HT-29, A549, SW620, HCT-15, HCT116, MDA-MB-231, MCF7, SK-OV-3, PANC-1, AsPc-1, THP-1, Huh7, KG-1, RAJI, HB-CB, Jurkat, K562, CRL5826, CHO, MDCK, and Renca), an embryonic stem cell line (e.g., H1, H9, WIBR2, WIBR3, G-Olig2, ESF158, RW. 4, R1, and D3) and differentiated cells thereof, or an induced pluripotent stem cell line and differentiated cells thereof.

According to an embodiment of the present application, the use of the transposase described in the present application, the nucleic acid encoding the transposase described in the present application, the nucleic acid described in the present application, the nucleic acid construct described in the present application, the nucleic acid set described in the present application, the nucleic acid set construct described in the present application, the composition described in the present application, the recombinant vector described in the present application, or the recombinant host cell described in the present application for introducing an exogenous nucleic acid fragment into the genome of a host cell can be provided. The host cell can be any host cell in which transposases can be used. In some embodiments, the host cell includes, but is not limited to, an animal cell, a plant cell, an algal cell, a fungal cell, a yeast cell, or a bacterial cell. In some embodiments, the host cell includes a mammalian cell. In some embodiments, the host cell includes a primary cell (e.g., a mesenchymal stem cell, an endothelial cell, an epithelial cell, a fibroblast, a keratinocyte, a melanocyte, a smooth muscle cell, and an immune cell), an immortalized cell line (e.g., HEK293, NIH-3T3, RAW-264.7, STO, VERO, CT26, hTERT immortalized human endothelial/epithelial/fibroblast/keratinocyte/ductal/cell lines), a cancer cell line (e.g., Hela, HepG2/3, HL-60, HT-1080, HT-29, A549, SW620, HCT-15, HCT116, MDA-MB-231, MCF7, SK-OV-3, PANC-1, AsPc-1, THP-1, Huh7, KG-1, RAJI, HB-CB, Jurkat, K562, CRL5826, CHO, MDCK, and Renca), an embryonic stem cell line (e.g., H1, H9, WIBR2, WIBR3, G-Olig2, ESF158, RW. 4, R1, and D3) and differentiated cells thereof, or an induced pluripotent stem cell line and differentiated cells thereof.

According to an embodiment of the present application, the use of the transposase described in the present application, the nucleic acid encoding the transposase described in the present application, the nucleic acid described in the present application, the nucleic acid construct described in the present application, the nucleic acid set described in the present application, the nucleic acid set construct described in the present application, the composition described in the present application, the recombinant vector described in the present application, or the recombinant host cell described in the present application for preparing a drug or a preparation for gene therapy, cell therapy, genome research, or stem cell induction and post-induction differentiation can be provided.

The above various embodiments and preferences for the present application can be combined with each other (as long as they are not inherently contradictory to each other) and are suitable for the use of the present application, and the various embodiments formed by such combinations are considered as a part of the present application.

### EXAMPLES

Exemplary embodiments of the present application are described below in conjunction with the accompanying drawings, where various details of the examples of the present application are included to facilitate understanding. It should be understood that they are considered to be exemplary only and not intended to limit the protection scope of the present application. The protection scope of the present application is only defined by the claims. Therefore, those of ordinary skill in the art should be aware that various changes and modifications can be made to the examples described herein, without departing from the scope of the present application. Likewise, for clarity and conciseness, the description of well-known functions and structures is omitted in the following description.

Unless otherwise stated, the reagents and instruments used in the following examples are conventional products that are commercially available. Unless otherwise stated, experiments are performed under conventional conditions or conditions recommended by the manufacturer.

### Example 1: Construction of Transposon Activity Detection System

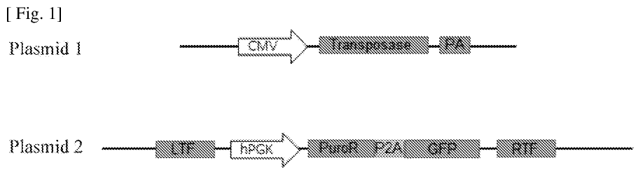

A set of detection system of a fluorescence-based reporter gene combined with an antibiotic screening marker was established to verify the activity of candidate transposons. The system performed verification using two plasmid vectors, as shown in FIG. 1: plasmid 1 was a plasmid expressing a transposase (Tn), comprising a constitutive promoter CMV (sequence as shown in SEQ ID NO: 298) that can initiate transcription in an eukaryotic cell, a sequence of a candidate transposase (as shown in Table 1), and a poly (A) sequence (PA, sequence as shown in SEQ ID NO: 299) that terminates transcription; and plasmid 2 was a transposon donor plasmid, comprising a GFP gene (sequence as shown in SEQ ID NO: 300), a puromycin resistance screening gene (PuroR, sequence as shown in SEQ ID NO: 301), promoter PGK (sequence as shown in SEQ ID NO: 302), P2A (sequence as shown in SEQ ID NO: 303) and a poly (A) element (sequence as shown in SEQ ID NO: 299), wherein transposon sequences (LTF and RTF in FIG. 1, sequences as shown in Table 1) that can be specifically recognized by the transposase were inserted at both ends of these sequences.

TABLE 1
Plasmid construction related sequences

| Name | Plasmid 1 | Plasmid 2 |
|---|---|---|
| TCM_A_A2 | SEQ ID NO: 1 | LTF (SEQ ID NO: 80), RTF (SEQ ID NO: 159) |
| TCM_A_B2 | SEQ ID NO: 2 | LTF (SEQ ID NO: 81), RTF (SEQ ID NO: 160) |
| TCM_A_B5 | SEQ ID NO: 3 | LTF (SEQ ID NO: 82), RTF (SEQ ID NO: 161) |
| TCM_A_C6 | SEQ ID NO: 4 | LTF (SEQ ID NO: 83), RTF (SEQ ID NO: 162) |
| TCM_A_C9 | SEQ ID NO: 5 | LTF (SEQ ID NO: 84), RTF (SEQ ID NO: 163) |
| TCM_A_D1 | SEQ ID NO: 6 | LTF (SEQ ID NO: 85), RTF (SEQ ID NO: 164) |
| TCM_A_D3 | SEQ ID NO: 7 | LTF (SEQ ID NO: 86), RTF (SEQ ID NO: 165) |
| TCM_A_D4 | SEQ ID NO: 8 | LTF (SEQ ID NO: 87), RTF (SEQ ID NO: 166) |
| TCM_A_F8 | SEQ ID NO: 9 | LTF (SEQ ID NO: 88), RTF (SEQ ID NO: 167) |
| TCM_A_G6 | SEQ ID NO: 10 | LTF (SEQ ID NO: 89), RTF (SEQ ID NO: 168) |
| TCM_B_C4 | SEQ ID NO: 11 | LTF (SEQ ID NO: 90), RTF (SEQ ID NO: 169) |
| TCM_B_C10 | SEQ ID NO: 12 | LTF (SEQ ID NO: 91), RTF (SEQ ID NO: 170) |
| TCM_B_C11 | SEQ ID NO: 13 | LTF (SEQ ID NO: 92), RTF (SEQ ID NO: 171) |
| TCM_B_C12 | SEQ ID NO: 14 | LTF (SEQ ID NO: 93), RTF (SEQ ID NO: 172) |
| TCM_B_D1 | SEQ ID NO: 15 | LTF (SEQ ID NO: 94), RTF (SEQ ID NO: 173) |
| TCM_B_D4 | SEQ ID NO: 16 | LTF (SEQ ID NO: 95), RTF (SEQ ID NO: 174) |
| TCM_B_D5 | SEQ ID NO: 17 | LTF (SEQ ID NO: 96), RTF (SEQ ID NO: 175) |
| TCM_B_D6 | SEQ ID NO: 18 | LTF (SEQ ID NO: 97), RTF (SEQ ID NO: 176) |
| TCM_B_D7 | SEQ ID NO: 19 | LTF (SEQ ID NO: 98), RTF (SEQ ID NO: 177) |
| TCM_B_D8 | SEQ ID NO: 20 | LTF (SEQ ID NO: 99), RTF (SEQ ID NO: 178) |
| TCM_B_D10 | SEQ ID NO: 21 | LTF (SEQ ID NO: 100), RTF (SEQ ID NO: 179) |
| TCM_B_E1 | SEQ ID NO: 22 | LTF (SEQ ID NO: 101), RTF (SEQ ID NO: 180) |
| TCM_B_E4 | SEQ ID NO: 23 | LTF (SEQ ID NO: 102), RTF (SEQ ID NO: 181) |
| TCM_B_E6 | SEQ ID NO: 24 | LTF (SEQ ID NO: 103), RTF (SEQ ID NO: 182) |
| TCM_B_F2 | SEQ ID NO: 25 | LTF (SEQ ID NO: 104), RTF (SEQ ID NO: 183) |
| TCM_B_F3 | SEQ ID NO: 26 | LTF (SEQ ID NO: 105), RTF (SEQ ID NO: 184) |
| TCM_B_F5 | SEQ ID NO: 27 | LTF (SEQ ID NO: 106), RTF (SEQ ID NO: 185) |
| TCM_B_F10 | SEQ ID NO: 28 | LTF (SEQ ID NO: 107), RTF (SEQ ID NO: 186) |
| TCM_B_F11 | SEQ ID NO: 29 | LTF (SEQ ID NO: 108), RTF (SEQ ID NO: 187) |
| TCM_B_G2 | SEQ ID NO: 30 | LTF (SEQ ID NO: 109), RTF (SEQ ID NO: 188) |
| TCM_B_G10 | SEQ ID NO: 31 | LTF (SEQ ID NO: 110), RTF (SEQ ID NO: 189) |
| TCM_B_G11 | SEQ ID NO: 32 | LTF (SEQ ID NO: 111), RTF (SEQ ID NO: 190) |
| TCM_C_A12 | SEQ ID NO: 33 | LTF (SEQ ID NO: 112), RTF (SEQ ID NO: 191) |
| TCM_C_B2 | SEQ ID NO: 34 | LTF (SEQ ID NO: 113), RTF (SEQ ID NO: 192) |
| TCM_C_B8 | SEQ ID NO: 35 | LTF (SEQ ID NO: 114), RTF (SEQ ID NO: 193) |
| TCM_C_C3 | SEQ ID NO: 36 | LTF (SEQ ID NO: 115), RTF (SEQ ID NO: 194) |
| TCM_D_B11 | SEQ ID NO: 37 | LTF (SEQ ID NO: 116), RTF (SEQ ID NO: 195) |
| TCM_D_G9 | SEQ ID NO: 38 | LTF (SEQ ID NO: 117), RTF (SEQ ID NO: 196) |
| TCM_E_A1 | SEQ ID NO: 39 | LTF (SEQ ID NO: 118), RTF (SEQ ID NO: 197) |
| TCM_E_A4 | SEQ ID NO: 40 | LTF (SEQ ID NO: 119), RTF (SEQ ID NO: 198) |
| TCM_E_A5 | SEQ ID NO: 41 | LTF (SEQ ID NO: 120), RTF (SEQ ID NO: 199) |
| TCM_E_A6 | SEQ ID NO: 42 | LTF (SEQ ID NO: 121), RTF (SEQ ID NO: 200) |
| TCM_E_A7 | SEQ ID NO: 43 | LTF (SEQ ID NO: 122), RTF (SEQ ID NO: 201) |
| TCM_E_A8 | SEQ ID NO: 44 | LTF (SEQ ID NO: 123), RTF (SEQ ID NO: 202) |
| TCM_E_B6 | SEQ ID NO: 45 | LTF (SEQ ID NO: 124), RTF (SEQ ID NO: 203) |
| TCM_E_B8 | SEQ ID NO: 46 | LTF (SEQ ID NO: 125), RTF (SEQ ID NO: 204) |
| TCM_E_B10 | SEQ ID NO: 47 | LTF (SEQ ID NO: 126), RTF (SEQ ID NO: 205) |
| TCM_E_B11 | SEQ ID NO: 48 | LTF (SEQ ID NO: 127), RTF (SEQ ID NO: 206) |
| TCM_E_B12 | SEQ ID NO: 49 | LTF (SEQ ID NO: 128), RTF (SEQ ID NO: 207) |
| TCM_E_C1 | SEQ ID NO: 50 | LTF (SEQ ID NO: 129), RTF (SEQ ID NO: 208) |
| TCM_E_C2 | SEQ ID NO: 51 | LTF (SEQ ID NO: 130), RTF (SEQ ID NO: 209) |
| TCM_E_C3 | SEQ ID NO: 52 | LTF (SEQ ID NO: 131), RTF (SEQ ID NO: 210) |
| TCM_E_C5 | SEQ ID NO: 53 | LTF (SEQ ID NO: 132), RTF (SEQ ID NO: 211) |
| TCM_E_C6 | SEQ ID NO: 54 | LTF (SEQ ID NO: 133), RTF (SEQ ID NO: 212) |
| TCM_E_C8 | SEQ ID NO: 55 | LTF (SEQ ID NO: 134), RTF (SEQ ID NO: 213) |
| TCM_E_C10 | SEQ ID NO: 56 | LTF (SEQ ID NO: 135), RTF (SEQ ID NO: 214) |
| TCM_E_C11 | SEQ ID NO: 57 | LTF (SEQ ID NO: 136), RTF (SEQ ID NO: 215) |
| TCM_E_C12 | SEQ ID NO: 58 | LTF (SEQ ID NO: 137), RTF (SEQ ID NO: 216) |
| TCM_E_D6 | SEQ ID NO: 59 | LTF (SEQ ID NO: 138), RTF (SEQ ID NO: 217) |
| TCM_E_D7 | SEQ ID NO: 60 | LTF (SEQ ID NO: 139), RTF (SEQ ID NO: 218) |
| TCM_E_D12 | SEQ ID NO: 61 | LTF (SEQ ID NO: 140), RTF (SEQ ID NO: 219) |
| TCM_E_E6 | SEQ ID NO: 62 | LTF (SEQ ID NO: 141), RTF (SEQ ID NO: 220) |
| TCM_E_E7 | SEQ ID NO: 63 | LTF (SEQ ID NO: 142), RTF (SEQ ID NO: 221) |
| TCM_E_E8 | SEQ ID NO: 64 | LTF (SEQ ID NO: 143), RTF (SEQ ID NO: 222) |
| TCM_E_E10 | SEQ ID NO: 65 | LTF (SEQ ID NO: 144), RTF (SEQ ID NO: 223) |
| TCM_E_E11 | SEQ ID NO: 66 | LTF (SEQ ID NO: 145), RTF (SEQ ID NO: 224) |
| TCM_E_E12 | SEQ ID NO: 67 | LTF (SEQ ID NO: 146), RTF (SEQ ID NO: 225) |
| TCM_E_F2 | SEQ ID NO: 68 | LTF (SEQ ID NO: 147), RTF (SEQ ID NO: 226) |
| TCM_E_F3 | SEQ ID NO: 69 | LTF (SEQ ID NO: 148), RTF (SEQ ID NO: 227) |
| TCM_E_F5 | SEQ ID NO: 70 | LTF (SEQ ID NO: 149), RTF (SEQ ID NO: 228) |
| TCM_E_F7 | SEQ ID NO: 71 | LTF (SEQ ID NO: 150), RTF (SEQ ID NO: 229) |
| TCM_E_F8 | SEQ ID NO: 72 | LTF (SEQ ID NO: 151), RTF (SEQ ID NO: 230) |
| TCM_E_F11 | SEQ ID NO: 73 | LTF (SEQ ID NO: 152), RTF (SEQ ID NO: 231) |
| TCM_E_G4 | SEQ ID NO: 74 | LTF (SEQ ID NO: 153), RTF (SEQ ID NO: 232) |
| TCM_E_G5 | SEQ ID NO: 75 | LTF (SEQ ID NO: 154), RTF (SEQ ID NO: 233) |
| TCM_E_G6 | SEQ ID NO: 76 | LTF (SEQ ID NO: 155), RTF (SEQ ID NO: 234) |
| TCM_E_G7 | SEQ ID NO: 77 | LTF (SEQ ID NO: 156), RTF (SEQ ID NO: 235) |
| TCM_E_G9 | SEQ ID NO: 78 | LTF (SEQ ID NO: 157), RTF (SEQ ID NO: 236) |
| TCM_E_G12 | SEQ ID NO: 79 | LTF (SEQ ID NO: 158), RTF (SEQ ID NO: 237) |
| TCM_A_B8 | SEQ ID NO: 238 | LTF (SEQ ID NO: 258), RTF (SEQ ID NO: 278) |
| TCM_A_D6 | SEQ ID NO: 239 | LTF (SEQ ID NO: 259), RTF (SEQ ID NO: 279) |
| TCM_A_F7 | SEQ ID NO: 240 | LTF (SEQ ID NO: 260), RTF (SEQ ID NO: 280) |
| TCM_B_A8 | SEQ ID NO: 241 | LTF (SEQ ID NO: 261), RTF (SEQ ID NO: 281) |
| TCM_B_B8 | SEQ ID NO: 242 | LTF (SEQ ID NO: 262), RTF (SEQ ID NO: 282) |
| TCM_B_G5 | SEQ ID NO: 243 | LTF (SEQ ID NO: 263), RTF (SEQ ID NO: 283) |
| TCM_C_A8 | SEQ ID NO: 244 | LTF (SEQ ID NO: 264), RTF (SEQ ID NO: 284) |
| TCM_C_A11 | SEQ ID NO: 245 | LTF (SEQ ID NO: 265), RTF (SEQ ID NO: 285) |
| TCM_C_B11 | SEQ ID NO: 246 | LTF (SEQ ID NO: 266), RTF (SEQ ID NO: 286) |
| TCM_C_D2 | SEQ ID NO: 247 | LTF (SEQ ID NO: 267), RTF (SEQ ID NO: 287) |
| TCM_C_D8 | SEQ ID NO: 248 | LTF (SEQ ID NO: 268), RTF (SEQ ID NO: 288) |
| TCM_D_G3 | SEQ ID NO: 249 | LTF (SEQ ID NO: 269), RTF (SEQ ID NO: 289) |
| TCM_D_G8 | SEQ ID NO: 250 | LTF (SEQ ID NO: 270), RTF (SEQ ID NO: 290) |
| TCM_E_D4 | SEQ ID NO: 251 | LTF (SEQ ID NO: 271), RTF (SEQ ID NO: 291) |
| TCM_E_D5 | SEQ ID NO: 252 | LTF (SEQ ID NO: 272), RTF (SEQ ID NO: 292) |
| TCM_E_E4 | SEQ ID NO: 253 | LTF (SEQ ID NO: 273), RTF (SEQ ID NO: 293) |
| TCM_E_F4 | SEQ ID NO: 254 | LTF (SEQ ID NO: 274), RTF (SEQ ID NO: 294) |
| TCM_E_G1 | SEQ ID NO: 255 | LTF (SEQ ID NO: 275), RTF (SEQ ID NO: 295) |
| TCM_E_G2 | SEQ ID NO: 256 | LTF (SEQ ID NO: 276), RTF (SEQ ID NO: 296) |
| TCM_E_G3 | SEQ ID NO: 257 | LTF (SEQ ID NO: 277), RTF (SEQ ID NO: 297) |

When both plasmids were co-transfected into HEK293T cells, the transcription of the transposase gene from plasmid 1 was initiated to express a transposase protein, the transposase protein then recognized and bound to the transposon recognition sequences on plasmid 2, and cut all the sequences including the transposon recognition sequences, the GFP gene and the puromycin resistance gene from the plasmid vector and integrated them into the genome of a cell. When the cells were continuously cultured with a medium containing a certain concentration of puromycin, only the cells in which the transposition event occurred survived because they contained the puromycin resistance gene in their genome. The transposition activity level of the candidate transposase was reflected by the number of surviving cells or their ability to form monoclonal cells.

### Method for DNA Synthesis and Plasmid Construction:

Construction of plasmid 1: a DNA sequence corresponding to the amino acid sequence of the transposase was synthesized by Beijing Tsingke Biotech Co., Ltd. and GENERAL Biosystems (Anhui) Co., Ltd., and cloned into a plasmid vector pICOZ that contains a CMV promoter element via EcoRI site at the 5′ end and NotI site at the 3′ end, so that the transposase gene is transcribed and subsequently translated into a functional protein in eukaryotic cells under the control of the CMV promoter.

Construction of plasmid 2: the transposon sequences (including terminal inverted repeats (TIR)) are located at both sides of the open reading frame of the transposase, the left transposon fragment (LTF) comprises all DNA sequences from the target site duplication (TSD) sequence at the 5′ end to the sequence before the transposase start codon, and the right transposon fragment (RTF) comprises all DNA sequences from the first base after the transposase stop codon to the TSD sequence at the 3′ end. In principle, the terminal repeats at both sides recognized by the transposase are comprised in the transposon sequences at both sides, respectively. LTF and RTF sequences were synthesized by BGI Tech Solutions (Beijing Liuhe) Co., Ltd., and were respectively cloned into a pMV plasmid vector that contains elements such as a PGK promoter, a puromycin resistance gene (PuroR), P2A, a green fluorescent protein (GFP) gene and poly (A), so that LTF was located upstream of the PGK promoter and RTF was located downstream of the poly (A).

Plasmid 1 corresponds to plasmid 2 one by one.

### Example 2: High Throughput Screening of Transposition Activity

### 2.1 Cell Treatment (Day 0):

A HEK293T cells (commercially purchased) stably expressing the firefly luciferase gene were established for high throughput screening assay. When cultured to the logarithmic growth phase, the cells were digested and dispersed into single cells with 0.25% Trypsin (Thermo), and added to a 96-well cell culture plate pre-coated with PDL (Sigma) at a cell concentration of 1.0×10⁴ cells/well, and cultured overnight at 37° C. in 5% CO₂.

### 2.2 Cell Transfection (Day 1):

Two plasmids corresponding to each transposon system were mixed at a dose of 20 ng for plasmid 1 and 10 ng for plasmid 2, then mixed with a transfection reagent Lipofectamine 2000 (Thermo) at a ratio of the mass of the transfection plasmid (μg): the volume of the transfection reagent (L) being 1:2, and left to stand at room temperature for 15 min to form a transfection complex. The transfection complex was transferred to the cell culture plate and incubated with the cells, and two parallel tests were performed for each sample to be screened.

### 2.3 Cell Screening (Day 3)

48 h after transfection, the culture medium was replaced to DMEM (Thermo) screening medium containing 2 g/mL puromycin (Invivogen), 10% fetal bovine serum and 1% penicillin/streptomycin (Thermo), and cultured for 4 days at 37° C. in 5% CO₂. Then, the cells were digested into single cells with 0.25% Trypsin, diluted at a ratio of 1:5, transferred to another 96-well culture plate pre-coated with PDL, and cultured for 4 days at 37° C. in a DMEM screening medium containing 2 μg/mL puromycin, 10% fetal bovine serum and 1% penicillin/streptomycin.

### 2.4 Detection of Cell Viability (Day 11)

2.4.1 Preparation of detection reagents: The Steady-Glo® Luciferase Assay System (Promega) was mixed with PBS at volume ratio of 1:5. The detection reagents was prepared at a dose of 50 μL/well, and 5 mL of detection reagents was prepared for a 96-well plate.

2.4.2 The cells screened by puromycin for 8 days were removed from the incubator. After the culture medium were removed, the detection reagent was added at a dose of 50 μL/well. After incubated at room temperature for 5 minutes in the dark, a multifunctional microplate reader with luminescence detection function was used for detection. The more cells survived after puromycin screening, the stronger the luminescence signal detected, indicating the higher transposition activity of the sample.

### 2.5 Statistical Results

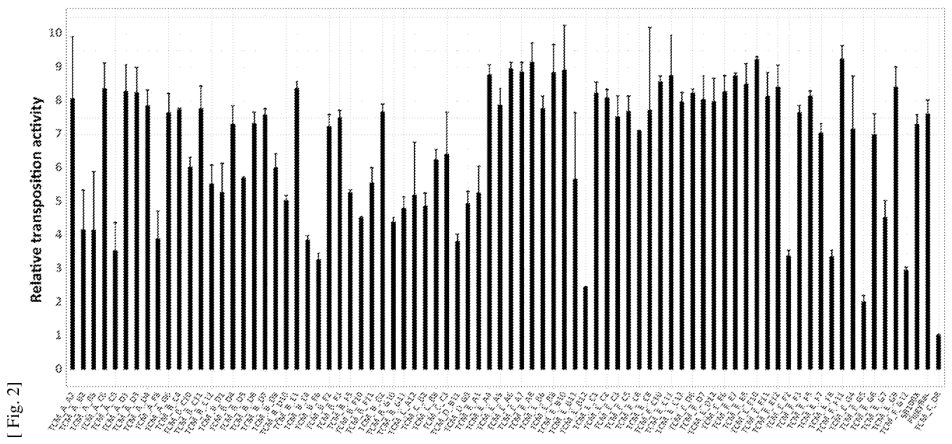

During high-throughput screening, positive and negative control were set up on each plate. According to the reading value of the luminescence signal detected by the microplate reader in each well, the fold change of the reading value of each sample (including the positive control) relative to the average reading value of the negative controls is calculated. The level of the calculated value indicates the level of the transposition activity. The statistical results of the relative transposition activity of the transposases of the present application compared to that of SB100X, PiggyBac and inactive transposases were as shown in FIG. 2 and Table 2.

TABLE 2
The results of relative transposition activity in example 2

| | Relative transposition activity |
|---|---|
| Name | (mean ± standard deviation) |
| TCM_A_A2 | 8.06 ± 1.83 |
| TCM_A_B2 | 4.16 ± 1.18 |
| TCM_A_B5 | 4.14 ± 1.74 |
| TCM_A_C6 | 8.35 ± 0.77 |
| TCM_A_C9 | 3.54 ± 0.82 |
| TCM_A_D1 | 8.27 ± 0.80 |
| TCM_A_D3 | 8.25 ± 0.74 |
| TCM_A_D4 | 7.85 ± 0.46 |
| TCM_A_F8 | 3.89 ± 0.82 |
| TCM_A_G6 | 7.65 ± 0.57 |
| TCM_B_C4 | 7.73 ± 0.05 |
| TCM_B_C10 | 6.03 ± 0.28 |
| TCM_B_C11 | 7.77 ± 0.66 |
| TCM_B_C12 | 5.53 ± 0.56 |
| TCM_B_D1 | 5.28 ± 0.86 |
| TCM_B_D4 | 7.31 ± 0.54 |
| TCM_B_D5 | 5.70 ± 0.03 |
| TCM_B_D6 | 7.33 ± 0.33 |
| TCM_B_D7 | 7.57 ± 0.20 |
| TCM_B_D8 | 6.01 ± 0.41 |
| TCM_B_D10 | 5.03 ± 0.15 |
| TCM_B_E1 | 8.36 ± 0.21 |
| TCM_B_E4 | 3.85 ± 0.13 |
| TCM_B_E6 | 3.27 ± 0.18 |
| TCM_B_F2 | 7.23 ± 0.35 |
| TCM_B_F3 | 7.50 ± 0.21 |
| TCM_B_F5 | 5.26 ± 0.09 |
| TCM_B_F10 | 4.51 ± 0.03 |
| TCM_B_F11 | 5.56 ± 0.45 |
| TCM_B_G2 | 7.67 ± 0.23 |
| TCM_B_G10 | 4.38 ± 0.14 |
| TCM_B_G11 | 4.79 ± 0.35 |
| TCM_C_A12 | 5.19 ± 1.57 |
| TCM_C_B2 | 4.87 ± 0.37 |
| TCM_C_B8 | 6.24 ± 0.30 |
| TCM_C_C3 | 6.41 ± 1.25 |
| TCM_D_B11 | 3.81 ± 0.21 |
| TCM_D_G9 | 4.93 ± 0.37 |
| TCM_E_A1 | 5.26 ± 0.80 |
| TCM_E_A4 | 8.78 ± 0.29 |
| TCM_E_A5 | 7.87 ± 0.48 |
| TCM_E_A6 | 8.96 ± 0.18 |
| TCM_E_A7 | 8.85 ± 0.29 |
| TCM_E_A8 | 9.15 ± 0.57 |
| TCM_E_B6 | 7.77 ± 0.36 |
| TCM_E_B8 | 8.85 ± 0.82 |
| TCM_E_B10 | 8.93 ± 1.31 |
| TCM_E_B11 | 5.67 ± 1.97 |
| TCM_E_B12 | 2.45 ± 0.00 |
| TCM_E_C1 | 8.22 ± 0.33 |
| TCM_E_C2 | 8.09 ± 0.25 |
| TCM_E_C3 | 7.52 ± 0.62 |
| TCM_E_C5 | 7.69 ± 0.44 |
| TCM_E_C6 | 7.09 ± 0.03 |
| TCM_E_C8 | 7.73 ± 2.44 |
| TCM_E_C10 | 8.57 ± 0.17 |
| TCM_E_C11 | 8.75 ± 1.19 |
| TCM_E_C12 | 7.96 ± 0.27 |
| TCM_E_D6 | 8.23 ± 0.11 |
| TCM_E_D7 | 8.03 ± 0.71 |
| TCM_E_D12 | 7.98 ± 0.70 |
| TCM_E_E6 | 8.27 ± 0.48 |
| TCM_E_E7 | 8.76 ± 0.07 |
| TCM_E_E8 | 8.50 ± 0.61 |
| TCM_E_E10 | 9.24 ± 0.08 |
| TCM_E_E11 | 8.14 ± 0.70 |
| TCM_E_E12 | 8.40 ± 0.65 |
| TCM_E_F2 | 3.38 ± 0.16 |
| TCM_E_F3 | 7.65 ± 0.21 |
| TCM_E_F5 | 8.14 ± 0.14 |
| TCM_E_F7 | 7.05 ± 0.27 |
| TCM_E_F8 | 3.36 ± 0.18 |
| TCM_E_F11 | 9.25 ± 0.39 |
| TCM_E_G4 | 7.16 ± 1.57 |
| TCM_E_G5 | 2.01 ± 0.18 |
| TCM_E_G6 | 7.00 ± 0.61 |
| TCM_E_G7 | 4.52 ± 0.51 |
| TCM_E_G9 | 8.40 ± 0.61 |
| TCM_E_G12 | 2.95 ± 0.08 |
| SB100X | 7.30 ± 0.28 |
| PiggyBac | 7.60 ± 0.42 |
| TCM_C_D8 | 1.00 ± 0.04 |

### Example 3: Transposition Activity Assay

### 3.1 Cell Treatment (Day 0):

After HEK293T cells (commercially purchased) were cultured to the logarithmic growth phase, they were digested and dispersed into single cells with 0.25% Trypsin (Thermo), and added to a 24-well cell culture plate pre-coated with PDL (Sigma) at a cell concentration of 1.2×10⁵ cells/well, and cultured overnight at 37° C. in 5% CO₂.

### 3.2 Cell Transfection (Day 1):

Two plasmids corresponding to each transposon system were mixed at a dose of 200 ng for plasmid 1 and 100 ng for plasmid 2, then mixed with a transfection reagent Lipofectamine 2000 (Thermo) at a ratio of the mass of the transfection plasmid (μg): the volume of the transfection reagent (μL) being 1:2, and left to stand at room temperature for 15 min to form a transfection complex. The transfection complex was transferred to the cell culture plate and incubated with the cells, and two parallel tests were performed for each sample to be screened.

### 3.3 Cell Screening (Day 3)

48 h after transfection, the cells were digested and dispersed into single cells with 0.25% Trypsin, the cells were added to a DMEM (Thermo) screening medium containing 2 μg/mL puromycin (Invivogen), 10% fetal bovine serum and antibiotics (1% penicillin/streptomycin, Thermo), diluted at a ratio of 1:2000, and transferred to a 6-well culture plate for further culture. After 10 days of continuous screening culture with the puromycin resistance medium, the clones were counted, and the transposition activity of the transposase was calculated.

### 3.4 Cell Staining (Day 13)

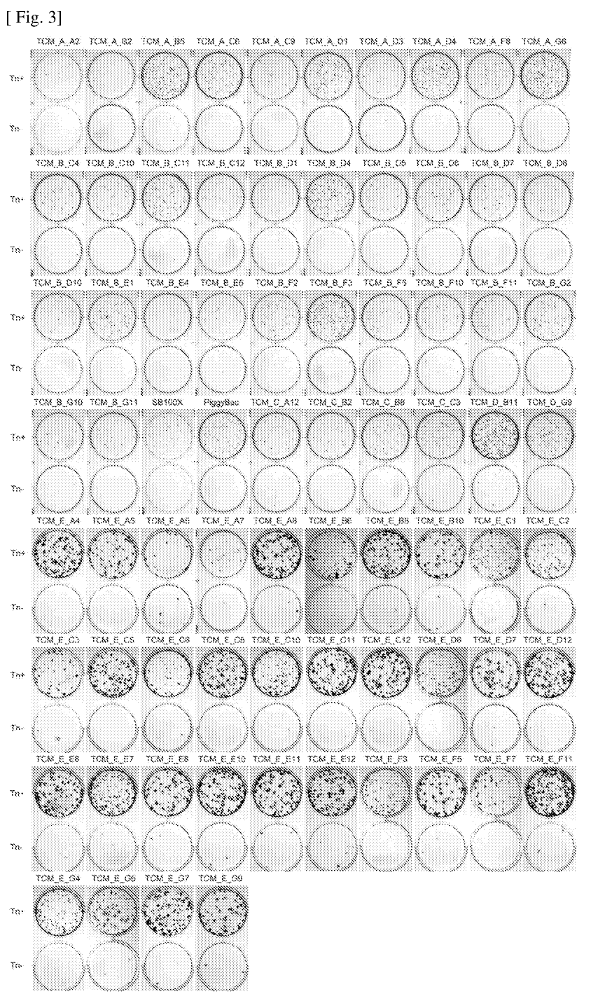

The cells that were screened by puromycin and cultured in the 6-well plate were washed with PBS, and then fixed at room temperature for 15 min with 4% paraformaldehyde. The waste liquid was discarded, and a 0.2% methylene blue staining solution was added to the cells. The cells were stained at room temperature for 1 h. The stained cell clones were washed with PBS, and photographed in an imaging system (BioRad). The number of cell clones in each well was counted. The cloning and screening results of the transposases of the present application in HEK293T cells were as shown in FIG. 3. The staining results of the surviving cell clones after puromycin resistance screening were as shown in the figures, showing that the transposition event occurred successfully. Tn+ represents co-transfection of a transposase plasmid and a donor plasmid, and Tn-represents transfection of a donor plasmid only, as a negative control for each transposase sample.

### 3.5 Statistical Results

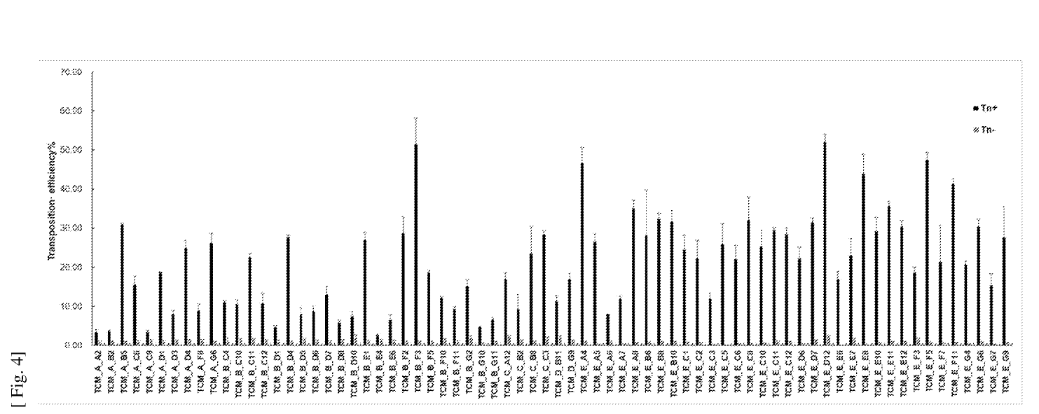

The statistical results of transposition activity were as shown in FIG. 4 and Table 3. Tn+ represents co-transfection of a transposase plasmid and a donor plasmid, and Tn-represents transfection of a donor plasmid only. The y-axis in the figure showed the percentage of transposition activity according to calculation, and the specific calculation formula was as follows: transposition efficiency (%)=the number of cell clones per well/(the number of cells plated per well×transfection efficiency (GFP positive cells %))×100%.

TABLE 3
The statistical results of transposition activity in example 3

| | Tn+ | Tn− |
|---|---|---|
| Name | (mean ± standard deviation) | (mean ± standard deviation) |
| TCM_A_A2 | 3.39 ± 0.63 | 0.72 ± 0.41 |
| TCM_A_B2 | 3.52 ± 0.33 | 1.03 ± 0.09 |
| TCM_A_B5 | 30.99 ± 0.32 | 0.95 ± 0.11 |
| TCM_A_C6 | 15.56 ± 2.23 | 0.86 ± 0.38 |
| TCM_A_C9 | 3.32 ± 0.50 | 1.25 ± 0.23 |
| TCM_A_D1 | 18.72 ± 0.11 | 0.97 ± 0.20 |
| TCM_A_D3 | 8.07 ± 0.89 | 1.10 ± 0.19 |
| TCM_A_D4 | 24.91 ± 1.96 | 1.32 ± 0.15 |
| TCM_A_F8 | 8.99 ± 1.64 | 1.19 ± 0.46 |
| TCM_A_G6 | 26.23 ± 2.46 | 0.73 ± 0.33 |
| TCM_B_C4 | 10.96 ± 0.57 | 1.04 ± 0.80 |
| TCM_B_C10 | 10.52 ± 1.17 | 1.38 ± 0.33 |
| TCM_B_C11 | 22.55 ± 0.95 | 1.31 ± 0.39 |
| TCM_B_C12 | 10.72 ± 2.71 | 1.21 ± 0.27 |
| TCM_B_D1 | 4.63 ± 0.49 | 0.82 ± 0.46 |
| TCM_B_D4 | 27.72 ± 0.49 | 0.88 ± 0.28 |
| TCM_B_D5 | 7.98 ± 1.63 | 1.26 ± 0.48 |
| TCM_B_D6 | 8.73 ± 1.33 | 0.87 ± 0.39 |
| TCM_B_D7 | 13.00 ± 2.21 | 0.73 ± 0.44 |
| TCM_B_D8 | 5.73 ± 0.69 | 1.14 ± 0.32 |
| TCM_B_D10 | 7.38 ± 1.29 | 1.89 ± 0.88 |
| TCM_B_E1 | 27.05 ± 1.85 | 1.14 ± 0.15 |
| TCM_B_E4 | 2.65 ± 0.25 | 1.24 ± 0.37 |
| TCM_B_E6 | 6.44 ± 1.32 | 1.06 ± 0.21 |
| TCM_B_F2 | 28.62 ± 4.29 | 0.84 ± 0.31 |
| TCM_B_F3 | 51.48 ± 6.81 | 1.01 ± 0.12 |
| TCM_B_F5 | 18.69 ± 0.52 | 1.02 ± 0.06 |
| TCM_B_F10 | 12.13 ± 0.30 | 1.28 ± 0.42 |
| TCM_B_F11 | 9.34 ± 0.63 | 0.75 ± 0.43 |
| TCM_B_G2 | 15.12 ± 1.90 | 2.02 ± 0.40 |
| TCM_B_G10 | 4.79 ± 0.02 | 0.47 ± 0.19 |
| TCM_B_G11 | 6.60 ± 0.54 | 0.76 ± 0.28 |
| TCM_C_A12 | 16.94 ± 1.67 | 2.15 ± 0.54 |
| TCM_C_B2 | 9.21 ± 3.81 | 1.03 ± 0.36 |
| TCM_C_B8 | 23.52 ± 6.98 | 0.88 ± 0.26 |
| TCM_C_C3 | 28.42 ± 0.98 | 1.63 ± 0.56 |
| TCM_D_B11 | 11.32 ± 1.38 | 2.05 ± 0.44 |
| TCM_D_G9 | 17.01 ± 1.35 | 1.22 ± 0.11 |
| TCM_E_A4 | 46.76 ± 3.94 | 0.88 ± 0.22 |
| TCM_E_A5 | 26.57 ± 1.93 | 0.12 ± 0.21 |
| TCM_E_A6 | 7.99 ± 0.11 | 0.89 ± 0.25 |
| TCM_E_A7 | 11.84 ± 0.72 | 0.31 ± 0.09 |
| TCM_E_A8 | 35.12 ± 2.07 | 0.64 ± 0.22 |
| TCM_E_B6 | 28.25 ± 11.54 | 0.76 ± 0.12 |
| TCM_E_B8 | 32.30 ± 1.58 | 0.94 ± 0.07 |
| TCM_E_B10 | 31.75 ± 2.78 | 0.90 ± 0.13 |
| TCM_E_C1 | 24.49 ± 3.73 | 0.78 ± 0.16 |
| TCM_E_C2 | 22.28 ± 4.74 | 0.56 ± 0.25 |
| TCM_E_C3 | 12.01 ± 1.39 | 0.14 ± 0.13 |
| TCM_E_C5 | 26.03 ± 5.16 | 0.05 ± 0.08 |
| TCM_E_C6 | 22.08 ± 3.58 | 0.45 ± 0.11 |
| TCM_E_C8 | 32.17 ± 5.82 | 0.41 ± 0.09 |
| TCM_E_C10 | 25.37 ± 4.17 | 0.49 ± 0.16 |
| TCM_E_C11 | 29.47 ± 0.79 | 0.86 ± 0.30 |
| TCM_E_C12 | 28.43 ± 1.61 | 0.87 ± 0.14 |
| TCM_E_D6 | 22.19 ± 3.00 | 0.30 ± 0.15 |
| TCM_E_D7 | 31.40 ± 1.19 | 1.31 ± 0.13 |
| TCM_E_D12 | 51.95 ± 2.16 | 2.18 ± 0.47 |
| TCM_E_E6 | 17.00 ± 1.96 | 0.04 ± 0.07 |
| TCM_E_E7 | 23.05 ± 4.30 | 1.40 ± 0.48 |
| TCM_E_E8 | 43.94 ± 5.08 | 0.18 ± 0.20 |
| TCM_E_E10 | 29.25 ± 3.51 | 0.60 ± 0.21 |
| TCM_E_E11 | 35.79 ± 1.09 | 0.88 ± 0.13 |
| TCM_E_E12 | 30.49 ± 1.58 | 0.90 ± 0.14 |
| TCM_E_F3 | 18.69 ± 1.30 | 1.28 ± 0.59 |
| TCM_E_F5 | 47.55 ± 1.78 | 0.61 ± 0.30 |
| TCM_E_F7 | 21.32 ± 9.39 | 0.28 ± 0.33 |
| TCM_E_F11 | 41.43 ± 1.20 | 0.74 ± 0.12 |
| TCM_E_G4 | 20.71 ± 0.80 | 0.37 ± 0.06 |
| TCM_E_G6 | 30.42 ± 1.90 | 0.83 ± 0.21 |
| TCM_E_G7 | 15.31 ± 3.06 | 0.22 ± 0.14 |
| TCM_E_G9 | 27.65 ± 7.87 | 0.60 ± 0.27 |

During the execution of all examples described above, two transposons, SB100X and PiggyBac, were used as positive controls for assessing the transposition activity of the transposases of the present application. These two transposons were commercially used DNA transposons that had been recently patented, and were synthesized and cloned into the corresponding plasmid vectors using the same method as that in example 1 with reference to sequences reported by Lajos Ma'te's et al. (Molecular evolution of a novel hyperactive Sleeping Beauty transposase enables robust stable gene transfer in vertebrates, Nature Genetics, 2009 41 (6): 753-761) and Cary, L. C. et al. (Transposon mutagenesis of baculoviruses: analysis of Trichoplusia ni transposon IFP2 insertions within the FP-locus of nuclear polyhedrosis viruses, Virology, 1989, 172 (1): 156-169).

The statistical results of the transposition activity of the transposases of the present application were as shown in FIG. 2 and FIG. 4. The above results showed that 79 transposases of the present application (TCM_A_A2, TCM_A_B2, TCM_A_B5, TCM_A_C6, TCM_A_C9, TCM_A_D1, TCM_A_D3, TCM_A_D4, TCM_A_F8, TCM_A_G6, TCM_B_C4, TCM_B_C10, TCM_B_C11, TCM_B_C12, TCM_B_D1, TCM_B_D4, TCM_B_D5, TCM_B_D6, TCM_B_D7, TCM_B_D8, TCM_B_D10, TCM_B_E1, TCM_B_E4, TCM_B_E6, TCM_B_F2, TCM_B_F3, TCM_B_F5, TCM_B_F10, TCM_B_F11, TCM_B_G2, TCM_B_G10, TCM_B_G11, TCM_C_A12, TCM_C_B2, TCM_C_B8, TCM_C_C3, TCM_D_B11, TCM_D_G9, TCM_E_A1, TCM_E_A4, TCM_E_A5, TCM_E_A6, TCM_E_A7, TCM_E_A8, TCM_E_B6, TCM_E_B8, TCM_E_B10, TCM_E_B11, TCM_E_B12, TCM_E_C1, TCM_E_C2, TCM_E_C3, TCM_E_C5, TCM_E_C6, TCM_E_C8, TCM_E_C10, TCM_E_C11, TCM_E_C12, TCM_E_D6, TCM_E_D7, TCM_E_D12, TCM_E_E6, TCM_E_E7, TCM_E_E8, TCM_E_E10, TCM_E_E11, TCM_E_E12, TCM_E_F2, TCM_E_F3, TCM_E_F5, TCM_E_F7, TCM_E_F8, TCM_E_F11, TCM_E_G4, TCM_E_G5, TCM_E_G6, TCM_E_G7, TCM_E_G9, TCM_E_G12) had good transposition activity.

Meanwhile, a large number of transposases with inactive or low transposition activity were also found during the screening process (e.g. TCM_A_B8, TCM_A_D6, TCM_A_F7, TCM_B_A8, TCM_B_B8, TCM_B_G5, TCM_C_A8, TCM_C_A11, TCM_C_B11, TCM_C_D2, TCM_C_D8, TCM_D_G3, TCM_D_G8, TCM_E_D4, TCM_E_D5, TCM_E_E4, TCM_E_F4, TCM_E_G1, TCM_E_G2, TCM_E_G3 in Table 1 of this application). Compared with these transposases with inactive or low transposition activity, the transposition activity of the 79 transposases of the present application were markedly higher (TCM_A_A2, TCM_A_B2, TCM_A_B5, TCM_A_C6, TCM_A_C9, TCM_A_D1, TCM_A_D3, TCM_A_D4, TCM_A_F8, TCM_A_G6, TCM_B_C4, TCM_B_C10, TCM_B_C11, TCM_B_C12, TCM_B_D1, TCM_B_D4, TCM_B_D5, TCM_B_D6, TCM_B_D7, TCM_B_D8, TCM_B_D10, TCM_B_E1, TCM_B_E4, TCM_B_E6, TCM_B_F2, TCM_B_F3, TCM_B_F5, TCM_B_F10, TCM_B_F11, TCM_B_G2, TCM_B_G10, TCM_B_G11 TCM_C_A12, TCM_C_B2, TCM_C_B8, TCM_C_C3, TCM_D_B11, TCM_D_G9, TCM_E_A1, TCM_E_A4, TCM_E_A5, TCM_E_A6, TCM_E_A7, TCM_E_A8, TCM_E_B6, TCM_E_B8, TCM_E_B10, TCM_E_B11, TCM_E_B12, TCM_E_C1, TCM_E_C2, TCM_E_C3, TCM_E_C5, TCM_E_C6, TCM_E_C8, TCM_E_C10, TCM_E_C11, TCM_E_C12, TCM_E_D6, TCM_E_D7, TCM_E_D12, TCM_E_E6, TCM_E_E7, TCM_E_E8, TCM_E_E10, TCM_E_E11, TCM_E_E12, TCM_E_F2, TCM_E_F3, TCM_E_F5, TCM_E_F7, TCM_E_F8, TCM_E_F11, TCM_E_G4, TCM_E_G5, TCM_E_G6, TCM_E_G7, TCM_E_G9, TCM_E_G12), and most of them were comparable to or better than that of SB100X and PiggyBac.

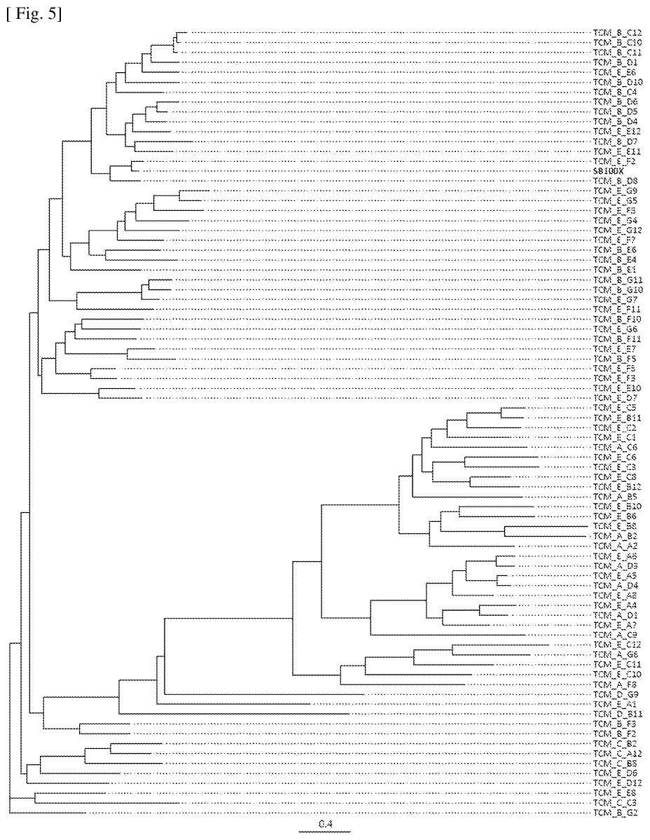

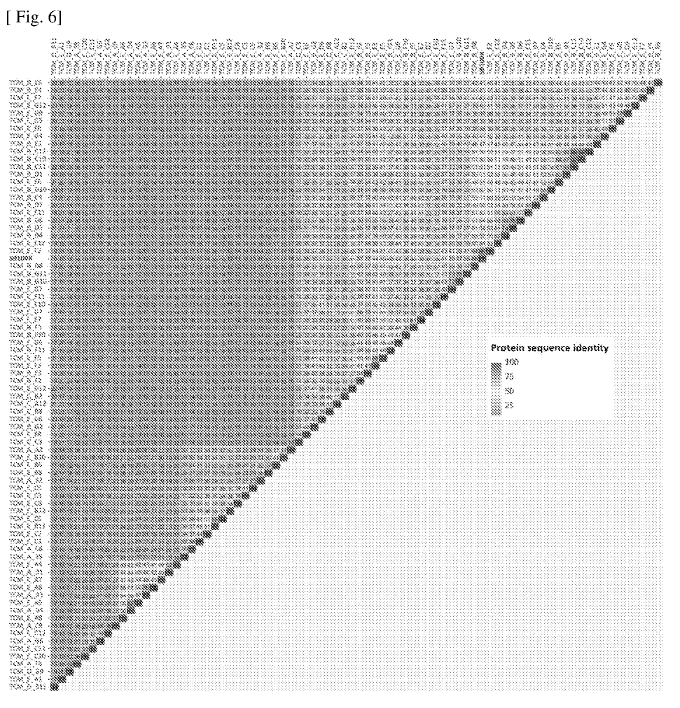

In addition, FIG. 5 showed an evolutionary branching diagram of the transposons of the Tc1/mariner superfamily in the present application based on protein sequences. FIG. 6 showed the protein sequence similarity (%) among the transposons of the Tc1/mariner superfamily in the present application. The results showed that these transposons covered different branches of the superfamily, and SB100X was also included.

It should be stated that the above are only the preferred examples of the present application and are not intended to limit the present application. For those of ordinary skill in the art, various modifications and changes can be made to the present application. Although the specific embodiments have been described, for the applicant or a person skilled in the art, the substitutions, modifications, changes, improvements, and substantial equivalents of the above embodiments may exist or cannot be foreseen currently. Therefore, the submitted appended claims and claims that may be modified are intended to cover all such substitutions, modifications, changes, improvements, and substantial equivalents. It is important that, as the technology evolves, many elements described herein may be replaced with equivalent elements that appear after the present application.

1. An isolated transposase, wherein the transposase has a transposase sequence selected from the following (i) or a variant sequence of the aforementioned transposase having a transposase activity in (ii)-(iv):
(i) at least one amino acid sequence as shown in any one of SEQ ID NOs: 1-79;
(ii) at least one of sequences obtained by performing deletion, substitution, insertion, or mutation of 1, 2, 3, 4, 5, 6, 7, 8, 9 or 10 amino acids on the amino acid sequence as shown in any one of SEQ ID NOs: 1-79;
(iii) at least one of amino acid sequences having at least 70%, 80%, 90%, 95% or 99% identity to the amino acid sequence as shown in any one of SEQ ID NOs: 1-79; and
(iv) at least one of sequences obtained by further fusing the amino acid sequence as shown in any one of SEQ ID NOs: 1-79 with other sequences.

2. The transposase according to claim 1, wherein the transposase has a transposase sequence selected from at least one of the following groups (1)-(8):
(1) at least one amino acid sequence as shown in any one of SEQ ID NOs: 11-36 and 59-79;
(2) at least one amino acid sequence as shown in any one of SEQ ID NOs: 3-4 and 48-55;
(3) at least one amino acid sequence as shown in any one of SEQ ID NOs: 5-8 and 40-44;
(4) at least one amino acid sequence as shown in any one of SEQ ID NOs: 1-2 and 45-47;
(5) at least one amino acid sequence as shown in any one of SEQ ID NOs: 9-10 and 56-58;
(6) at least one amino acid sequence as shown in any one of SEQ ID NOs: 38-39;
(7) at least one amino acid sequence as shown in SEQ ID NO: 37; and
(8) an amino acid sequence having at least 70% identity to any one of SEQ ID NOs: 1-79 in the aforementioned (1)-(7).

3. An isolated transposase, wherein the transposase comprises an amino acid sequence as shown in formula (I):
DE (I),
wherein
D is aspartic acid; and
E is glutamic acid.

4. An isolated transposase, wherein the transposase comprises an amino acid sequence as shown in formula (II):
D(X₁)ₐH (II),
wherein
D is aspartic acid;
H is histidine;
a is the number of amino acids; and
(X₁) is any amino acid, and a is 5.

5. An isolated transposase, wherein the transposase comprises an amino acid sequence as shown in formula (III):
P(X₂)(X₃) (III),
wherein
P is proline;
X₂ is any amino acid; and
X₃ is aspartic acid or glutamic acid.

6. An isolated transposase, wherein the transposase comprises at least two of the amino acid sequences as shown in formula (I), formula (II), and formula (III).

7. An isolated transposase, wherein the transposase comprises the amino acid sequences as shown in formula (I), formula (II), and formula (III).

8. The transposase according to claim 6, wherein formula (I) and formula (II) are spaced by 80 to 120 amino acids, and formula (II) and formula (III) are spaced by 20 to 40 amino acids.

9.-13. (canceled)

14. A nucleic acid, wherein the nucleic acid encodes the transposase according to claim 1.

15. A nucleic acid construct, comprising the nucleic acid according to claim 14, and further comprising a promoter and a poly(A) sequence.

16.-17. (canceled)

18. A nucleic acid set, comprising a 5′ recognition sequence, wherein the 5′ recognition sequence comprises at least one of the nucleotide sequences as shown in SEQ ID NOs: 80-158.

19. A nucleic acid set, comprising a 3′ recognition sequence, wherein the 3′ recognition sequence comprises at least one of the nucleotide sequences as shown in SEQ ID NOs: 159-237.

20. A nucleic acid set, comprising a 5′ recognition sequence and a 3′ recognition sequence, wherein the 5′ recognition sequence comprises the nucleotide sequence as shown in any one of SEQ ID NOs: 80-158 or a variant thereof, the 3′ recognition sequence comprises the nucleotide sequence as shown in any one of SEQ ID NOs: 159-237 or a variant thereof, and the nucleic acid set can be recognized by a specific transposase.

21. The nucleic acid set according to claim 20, wherein the 5′ recognition sequence or the 3′ recognition sequence comprises a terminal inverted repeat of at least one of 1-1200 nt, 1-800 nt, 1-600 nt, 1-400 nt, 1-200 nt, 1-100 nt, 5-80 nt, 10-70 nt, 20-60 nt, 20-700 nt, 50-600 nt, 100-500 nt, 150-400 nt, 150-300 nt, or 200-260 nt in length.

22. (canceled)

23. A nucleic acid set construct, comprising the nucleic acid set according to claim 20, and further comprising an exogenous nucleic acid fragment.

24. The nucleic acid set construct according to claim 23, wherein the exogenous nucleic acid fragment is operably inserted into the nucleic acid set construct through a polyclonal insertion site, and there may be one or more exogenous nucleic acid fragments, which may be the same or different; and a promoter can also be inserted to control the expression of the exogenous nucleic acid fragment.

25. The nucleic acid set construct according to claim 24, wherein the exogenous nucleic acid fragment comprises any gene of interest or any gene that is transposable.

26. The nucleic acid set construct according to claim 6, wherein the gene of a natural functional protein comprises a fluorescence-based reporter gene, a luciferase gene, or a resistance gene.

27.-29. (canceled)

30. The nucleic acid set construct according to claim 67, wherein the artificial chimeric gene comprises a gene of a chimeric antigen receptor.

31. (canceled)

32. A composition, wherein the composition comprises:
a Tc1/mariner superfamily transposase or a functional fragment thereof, or a nucleic acid encoding the Tc1/mariner superfamily transposase or the functional fragment thereof, wherein the transposase or the functional fragment thereof has a function of catalyzing the insertion of an exogenous nucleic acid fragment into the genome of a cell; and
a nucleic acid set, wherein the nucleic acid set can be recognized by a specific transposase or a functional fragment thereof.

33. The composition according to claim 32, wherein the composition is selected from at least one of the following groups (1)-(80), and any one of the following groups (1)-(79) comprises: a transposase-related sequence and a nucleic acid set,
(1) the transposase-related sequence is an amino acid sequence comprising the sequence as shown in SEQ ID NO: 1 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 80, and the 3′ recognition sequence is a nucleotide sequence comprising the sequence as shown in SEQ ID NO: 159;
(2) the transposase-related sequence is an amino acid sequence comprising the sequence as shown in SEQ ID NO: 2 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 81, and the 3′ recognition sequence is a nucleotide sequence comprising the sequence as shown in SEQ ID NO: 160;
(3) the transposase-related sequence is an amino acid sequence comprising the sequence as shown in SEQ ID NO: 3 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 82, and the 3′ recognition sequence is a nucleotide sequence comprising the sequence as shown in SEQ ID NO: 161;
(4) the transposase-related sequence is an amino acid sequence comprising the sequence as shown in SEQ ID NO: 4 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 83, and the 3′ recognition sequence is a nucleotide sequence comprising the sequence as shown in SEQ ID NO: 162;
(5) the transposase-related sequence is an amino acid sequence comprising the sequence as shown in SEQ ID NO: 5 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 84, and the 3′ recognition sequence is a nucleotide sequence comprising the sequence as shown in SEQ ID NO: 163;
(6) the transposase-related sequence is an amino acid sequence comprising the sequence as shown in SEQ ID NO: 6 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 85, and the 3′ recognition sequence is a nucleotide sequence comprising the sequence as shown in SEQ ID NO: 164;
(7) the transposase-related sequence is an amino acid sequence comprising the sequence as shown in SEQ ID NO: 7 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 86, and the 3′ recognition sequence is a nucleotide sequence comprising the sequence as shown in SEQ ID NO: 165;
(8) the transposase-related sequence is an amino acid sequence comprising the sequence as shown in SEQ ID NO: 8 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 87, and the 3′ recognition sequence is a nucleotide sequence comprising the sequence as shown in SEQ ID NO: 166;
(9) the transposase-related sequence is an amino acid sequence comprising the sequence as shown in SEQ ID NO: 9 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 88, and the 3′ recognition sequence is a nucleotide sequence comprising the sequence as shown in SEQ ID NO: 167;
(10) the transposase-related sequence is an amino acid sequence comprising the sequence as shown in SEQ ID NO: 10 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 89, and the 3′ recognition sequence is a nucleotide sequence comprising the sequence as shown in SEQ ID NO: 168;
(11) the transposase-related sequence is an amino acid sequence comprising the sequence as shown in SEQ ID NO: 11 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 90, and the 3′ recognition sequence is a nucleotide sequence comprising the sequence as shown in SEQ ID NO: 169;
(12) the transposase-related sequence is an amino acid sequence comprising the sequence as shown in SEQ ID NO: 12 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 91, and the 3′ recognition sequence is a nucleotide sequence comprising the sequence as shown in SEQ ID NO: 170;
(13) the transposase-related sequence is an amino acid sequence comprising the sequence as shown in SEQ ID NO: 13 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 92, and the 3′ recognition sequence is a nucleotide sequence comprising the sequence as shown in SEQ ID NO: 171;
(14) the transposase-related sequence is an amino acid sequence comprising the sequence as shown in SEQ ID NO: 14 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 93, and the 3′ recognition sequence is a nucleotide sequence comprising the sequence as shown in SEQ ID NO: 172;
(15) the transposase-related sequence is an amino acid sequence comprising the sequence as shown in SEQ ID NO: 15 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 94, and the 3′ recognition sequence is a nucleotide sequence comprising the sequence as shown in SEQ ID NO: 173;
(16) the transposase-related sequence is an amino acid sequence comprising the sequence as shown in SEQ ID NO: 16 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 95, and the 3′ recognition sequence is a nucleotide sequence comprising the sequence as shown in SEQ ID NO: 174;
(17) the transposase-related sequence is an amino acid sequence comprising the sequence as shown in SEQ ID NO: 17 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 96, and the 3′ recognition sequence is a nucleotide sequence comprising the sequence as shown in SEQ ID NO: 175;
(18) the transposase-related sequence is an amino acid sequence comprising the sequence as shown in SEQ ID NO: 18 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 97, and the 3′ recognition sequence is a nucleotide sequence comprising the sequence as shown in SEQ ID NO: 176;
(19) the transposase-related sequence is an amino acid sequence comprising the sequence as shown in SEQ ID NO: 19 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 98, and the 3′ recognition sequence is a nucleotide sequence comprising the sequence as shown in SEQ ID NO: 177;
(20) the transposase-related sequence is an amino acid sequence comprising the sequence as shown in SEQ ID NO: 20 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 99, and the 3′ recognition sequence is a nucleotide sequence comprising the sequence as shown in SEQ ID NO: 178;
(21) the transposase-related sequence is an amino acid sequence comprising the sequence as shown in SEQ ID NO: 21 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 100, and the 3′ recognition sequence is a nucleotide sequence comprising the sequence as shown in SEQ ID NO: 179;
(22) the transposase-related sequence is an amino acid sequence comprising the sequence as shown in SEQ ID NO: 22 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 101, and the 3′ recognition sequence is a nucleotide sequence comprising the sequence as shown in SEQ ID NO: 180;
(23) the transposase-related sequence is an amino acid sequence comprising the sequence as shown in SEQ ID NO: 23 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 102, and the 3′ recognition sequence is a nucleotide sequence comprising the sequence as shown in SEQ ID NO: 181;
(24) the transposase-related sequence is an amino acid sequence comprising the sequence as shown in SEQ ID NO: 24 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 103, and the 3′ recognition sequence is a nucleotide sequence comprising the sequence as shown in SEQ ID NO: 182;
(25) the transposase-related sequence is an amino acid sequence comprising the sequence as shown in SEQ ID NO: 25 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 104, and the 3′ recognition sequence is a nucleotide sequence comprising the sequence as shown in SEQ ID NO: 183;
(26) the transposase-related sequence is an amino acid sequence comprising the sequence as shown in SEQ ID NO: 26 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 105, and the 3′ recognition sequence is a nucleotide sequence comprising the sequence as shown in SEQ ID NO: 184;
(27) the transposase-related sequence is an amino acid sequence comprising the sequence as shown in SEQ ID NO: 27 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 106, and the 3′ recognition sequence is a nucleotide sequence comprising the sequence as shown in SEQ ID NO: 185;
(28) the transposase-related sequence is an amino acid sequence comprising the sequence as shown in SEQ ID NO: 28 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 107, and the 3′ recognition sequence is a nucleotide sequence comprising the sequence as shown in SEQ ID NO: 186;
(29) the transposase-related sequence is an amino acid sequence comprising the sequence as shown in SEQ ID NO: 29 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 108, and the 3′ recognition sequence is a nucleotide sequence comprising the sequence as shown in SEQ ID NO: 187;
(30) the transposase-related sequence is an amino acid sequence comprising the sequence as shown in SEQ ID NO: 30 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 109, and the 3′ recognition sequence is a nucleotide sequence comprising the sequence as shown in SEQ ID NO: 188;
(31) the transposase-related sequence is an amino acid sequence comprising the sequence as shown in SEQ ID NO: 31 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 110, and the 3′ recognition sequence is a nucleotide sequence comprising the sequence as shown in SEQ ID NO: 189;
(32) the transposase-related sequence is an amino acid sequence comprising the sequence as shown in SEQ ID NO: 32 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 111, and the 3′ recognition sequence is a nucleotide sequence comprising the sequence as shown in SEQ ID NO: 190;
(33) the transposase-related sequence is an amino acid sequence comprising the sequence as shown in SEQ ID NO: 33 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 112, and the 3′ recognition sequence is a nucleotide sequence comprising the sequence as shown in SEQ ID NO: 191;
(34) the transposase-related sequence is an amino acid sequence comprising the sequence as shown in SEQ ID NO: 34 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 113, and the 3′ recognition sequence is a nucleotide sequence comprising the sequence as shown in SEQ ID NO: 192;
(35) the transposase-related sequence is an amino acid sequence comprising the sequence as shown in SEQ ID NO: 35 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 114, and the 3′ recognition sequence is a nucleotide sequence comprising the sequence as shown in SEQ ID NO: 193;
(36) the transposase-related sequence is an amino acid sequence comprising the sequence as shown in SEQ ID NO: 36 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 115, and the 3′ recognition sequence is a nucleotide sequence comprising the sequence as shown in SEQ ID NO: 194;
(37) the transposase-related sequence is an amino acid sequence comprising the sequence as shown in SEQ ID NO: 37 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 116, and the 3′ recognition sequence is a nucleotide sequence comprising the sequence as shown in SEQ ID NO: 195;
(38) the transposase-related sequence is an amino acid sequence comprising the sequence as shown in SEQ ID NO: 38 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 117, and the 3′ recognition sequence is a nucleotide sequence comprising the sequence as shown in SEQ ID NO: 196;
(39) the transposase-related sequence is an amino acid sequence comprising the sequence as shown in SEQ ID NO: 39 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 118, and the 3′ recognition sequence is a nucleotide sequence comprising the sequence as shown in SEQ ID NO: 197;
(40) the transposase-related sequence is an amino acid sequence comprising the sequence as shown in SEQ ID NO: 40 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 119, and the 3′ recognition sequence is a nucleotide sequence comprising the sequence as shown in SEQ ID NO: 198;
(41) the transposase-related sequence is an amino acid sequence comprising the sequence as shown in SEQ ID NO: 41 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 120, and the 3′ recognition sequence is a nucleotide sequence comprising the sequence as shown in SEQ ID NO: 199;
(42) the transposase-related sequence is an amino acid sequence comprising the sequence as shown in SEQ ID NO: 42 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 121, and the 3′ recognition sequence is a nucleotide sequence comprising the sequence as shown in SEQ ID NO: 200;
(43) the transposase-related sequence is an amino acid sequence comprising the sequence as shown in SEQ ID NO: 43 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 122, and the 3′ recognition sequence is a nucleotide sequence comprising the sequence as shown in SEQ ID NO: 201;
(44) the transposase-related sequence is an amino acid sequence comprising the sequence as shown in SEQ ID NO: 44 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 123, and the 3′ recognition sequence is a nucleotide sequence comprising the sequence as shown in SEQ ID NO: 202;
(45) the transposase-related sequence is an amino acid sequence comprising the sequence as shown in SEQ ID NO: 45 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 124, and the 3′ recognition sequence is a nucleotide sequence comprising the sequence as shown in SEQ ID NO: 203;
(46) the transposase-related sequence is an amino acid sequence comprising the sequence as shown in SEQ ID NO: 46 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 125, and the 3′ recognition sequence is a nucleotide sequence comprising the sequence as shown in SEQ ID NO: 204;
(47) the transposase-related sequence is an amino acid sequence comprising the sequence as shown in SEQ ID NO: 47 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 126, and the 3′ recognition sequence is a nucleotide sequence comprising the sequence as shown in SEQ ID NO: 205;
(48) the transposase-related sequence is an amino acid sequence comprising the sequence as shown in SEQ ID NO: 48 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 127, and the 3′ recognition sequence is a nucleotide sequence comprising the sequence as shown in SEQ ID NO: 206;
(49) the transposase-related sequence is an amino acid sequence comprising the sequence as shown in SEQ ID NO: 49 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 128, and the 3′ recognition sequence is a nucleotide sequence comprising the sequence as shown in SEQ ID NO: 207;
(50) the transposase-related sequence is an amino acid sequence comprising the sequence as shown in SEQ ID NO: 50 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 129, and the 3′ recognition sequence is a nucleotide sequence comprising the sequence as shown in SEQ ID NO: 208;
(51) the transposase-related sequence is an amino acid sequence comprising the sequence as shown in SEQ ID NO: 51 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 130, and the 3′ recognition sequence is a nucleotide sequence comprising the sequence as shown in SEQ ID NO: 209;
(52) the transposase-related sequence is an amino acid sequence comprising the sequence as shown in SEQ ID NO: 52 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 131, and the 3′ recognition sequence is a nucleotide sequence comprising the sequence as shown in SEQ ID NO: 210;
(53) the transposase-related sequence is an amino acid sequence comprising the sequence as shown in SEQ ID NO: 53 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 132, and the 3′ recognition sequence is a nucleotide sequence comprising the sequence as shown in SEQ ID NO: 211;
(54) the transposase-related sequence is an amino acid sequence comprising the sequence as shown in SEQ ID NO: 54 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 133, and the 3′ recognition sequence is a nucleotide sequence comprising the sequence as shown in SEQ ID NO: 212;
(55) the transposase-related sequence is an amino acid sequence comprising the sequence as shown in SEQ ID NO: 55 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 134, and the 3′ recognition sequence is a nucleotide sequence comprising the sequence as shown in SEQ ID NO: 213;
(56) the transposase-related sequence is an amino acid sequence comprising the sequence as shown in SEQ ID NO: 56 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 135, and the 3′ recognition sequence is a nucleotide sequence comprising the sequence as shown in SEQ ID NO: 214;
(57) the transposase-related sequence is an amino acid sequence comprising the sequence as shown in SEQ ID NO: 57 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 136, and the 3′ recognition sequence is a nucleotide sequence comprising the sequence as shown in SEQ ID NO: 215;
(58) the transposase-related sequence is an amino acid sequence comprising the sequence as shown in SEQ ID NO: 58 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 137, and the 3′ recognition sequence is a nucleotide sequence comprising the sequence as shown in SEQ ID NO: 216;
(59) the transposase-related sequence is an amino acid sequence comprising the sequence as shown in SEQ ID NO: 59 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 138, and the 3′ recognition sequence is a nucleotide sequence comprising the sequence as shown in SEQ ID NO: 217;
(60) the transposase-related sequence is an amino acid sequence comprising the sequence as shown in SEQ ID NO: 60 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 139, and the 3′ recognition sequence is a nucleotide sequence comprising the sequence as shown in SEQ ID NO: 218;
(61) the transposase-related sequence is an amino acid sequence comprising the sequence as shown in SEQ ID NO: 61 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 140, and the 3′ recognition sequence is a nucleotide sequence comprising the sequence as shown in SEQ ID NO: 219;
(62) the transposase-related sequence is an amino acid sequence comprising the sequence as shown in SEQ ID NO: 62 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 141, and the 3′ recognition sequence is a nucleotide sequence comprising the sequence as shown in SEQ ID NO: 220;
(63) the transposase-related sequence is an amino acid sequence comprising the sequence as shown in SEQ ID NO: 63 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 142, and the 3′ recognition sequence is a nucleotide sequence comprising the sequence as shown in SEQ ID NO: 221;
(64) the transposase-related sequence is an amino acid sequence comprising the sequence as shown in SEQ ID NO: 64 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 143, and the 3′ recognition sequence is a nucleotide sequence comprising the sequence as shown in SEQ ID NO: 222;
(65) the transposase-related sequence is an amino acid sequence comprising the sequence as shown in SEQ ID NO: 65 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 144, and the 3′ recognition sequence is a nucleotide sequence comprising the sequence as shown in SEQ ID NO: 223;
(66) the transposase-related sequence is an amino acid sequence comprising the sequence as shown in SEQ ID NO: 66 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 145, and the 3′ recognition sequence is a nucleotide sequence comprising the sequence as shown in SEQ ID NO: 224;
(67) the transposase-related sequence is an amino acid sequence comprising the sequence as shown in SEQ ID NO: 67 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 146, and the 3′ recognition sequence is a nucleotide sequence comprising the sequence as shown in SEQ ID NO: 225;
(68) the transposase-related sequence is an amino acid sequence comprising the sequence as shown in SEQ ID NO: 68 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 147, and the 3′ recognition sequence is a nucleotide sequence comprising the sequence as shown in SEQ ID NO: 226;
(69) the transposase-related sequence is an amino acid sequence comprising the sequence as shown in SEQ ID NO: 69 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 148, and the 3′ recognition sequence is a nucleotide sequence comprising the sequence as shown in SEQ ID NO: 227;
(70) the transposase-related sequence is an amino acid sequence comprising the sequence as shown in SEQ ID NO: 70 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 149, and the 3′ recognition sequence is a nucleotide sequence comprising the sequence as shown in SEQ ID NO: 228;
(71) the transposase-related sequence is an amino acid sequence comprising the sequence as shown in SEQ ID NO: 71 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 150, and the 3′ recognition sequence is a nucleotide sequence comprising the sequence as shown in SEQ ID NO: 229;
(72) the transposase-related sequence is an amino acid sequence comprising the sequence as shown in SEQ ID NO: 72 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 151, and the 3′ recognition sequence is a nucleotide sequence comprising the sequence as shown in SEQ ID NO: 230;
(73) the transposase-related sequence is an amino acid sequence comprising the sequence as shown in SEQ ID NO: 73 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 152, and the 3′ recognition sequence is a nucleotide sequence comprising the sequence as shown in SEQ ID NO: 231;
(74) the transposase-related sequence is an amino acid sequence comprising the sequence as shown in SEQ ID NO: 74 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 153, and the 3′ recognition sequence is a nucleotide sequence comprising the sequence as shown in SEQ ID NO: 232;
(75) the transposase-related sequence is an amino acid sequence comprising the sequence as shown in SEQ ID NO: 75 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 154, and the 3′ recognition sequence is a nucleotide sequence comprising the sequence as shown in SEQ ID NO: 233;
(76) the transposase-related sequence is an amino acid sequence comprising the sequence as shown in SEQ ID NO: 76 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 155, and the 3′ recognition sequence is a nucleotide sequence comprising the sequence as shown in SEQ ID NO: 234;
(77) the transposase-related sequence is an amino acid sequence comprising the sequence as shown in SEQ ID NO: 77 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 156, and the 3′ recognition sequence is a nucleotide sequence comprising the sequence as shown in SEQ ID NO: 235;
(78) the transposase-related sequence is an amino acid sequence comprising the sequence as shown in SEQ ID NO: 78 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 157, and the 3′ recognition sequence is a nucleotide sequence comprising the sequence as shown in SEQ ID NO: 236;
(79) the transposase-related sequence is an amino acid sequence comprising the sequence as shown in SEQ ID NO: 79 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 158, and the 3′ recognition sequence is a nucleotide sequence comprising the sequence as shown in SEQ ID NO: 237; or
(80) a variant of any one of the aforementioned groups (1)-(79),
wherein the transposase-related sequence is the amino acid sequence of the variant of the transposase in each group or a nucleic acid sequence encoding the variant, and the variant has a variant sequence of the aforementioned transposase having a transposase activity selected from the following (i)-(iii):
(i) at least one of sequences obtained by performing deletion, substitution, insertion, or mutation of 1, 2, 3, 4, 5, 6, 7, 8, 9 or 10 amino acids on the amino acid sequence of the transposase in each group;
(ii) at least one of amino acid sequences having at least 70%, 80%, 90%, 95% or 99% identity to the amino acid sequence as shown in any one of SEQ ID NOs: 1-79; and
(iii) at least one of sequences obtained by further fusing the amino acid sequence as shown in any one of SEQ ID NOs: 1-79 with other sequences.

34. The composition according to claim 33, wherein the nucleic acid set further comprises a promoter and a poly(A) sequence.

35.-36. (canceled)

37. The composition according to claim 34, wherein the nucleic acid set further comprises an exogenous nucleic acid fragment.

38. The composition according to claim 37, wherein the exogenous nucleic acid fragment is operably inserted into the nucleic acid set through a polyclonal insertion site, and there may be one or more exogenous nucleic acid fragments, which may be the same or different; and a promoter can also be inserted to control the expression of the exogenous nucleic acid fragment.

39. The composition according to claim 38, wherein the exogenous nucleic acid fragment comprises any gene of interest or any gene that is transposable.

40. The composition according to claim 68, wherein the gene of a natural functional protein comprises a fluorescence-based reporter gene, a luciferase gene, or a resistance gene.

41.-43. (canceled)

44. The composition according to claim 68, wherein the artificial chimeric gene comprises a gene of a chimeric antigen receptor.

45. (canceled)

46. A recombinant vector, wherein the recombinant vector comprises the nucleic acid encoding the transposase according to claim 1.

47. The recombinant vector according to claim 46, wherein the recombinant vector comprises a recombinant cloning vector, a recombinant eukaryotic expression plasmid, or a recombinant viral vector.

48-49. (canceled)

50. A recombinant host cell, wherein the recombinant host cell comprises the transposase according to claim 1.

51. The recombinant host cell according to claim 50, wherein the recombinant host cell is an animal cell, a plant cell, an algal cell, a fungal cell, a yeast cell, or a bacterial cell.

52.-53. (canceled)

54. A method for introducing an exogenous nucleic acid fragment into the genome of a host cell, wherein the method comprises: delivering the transposase according to claim 1 or the nucleic acid encoding the transposase into a host cell.

55. A method for editing the genome of a host cell, wherein the method comprises: delivering the transposase according to claim 1 or the nucleic acid encoding the transposase into a host cell.

56. (canceled)

57. The method according to claim 55, wherein the delivery method comprises cationic liposome delivery, lipoid nanoparticulate delivery, cationic polymer delivery, vesicle-exosome delivery, gold nanoparticulate delivery, polypeptide and protein delivery, retrovirus delivery, lentivirus delivery, adenovirus delivery, adeno-associated virus delivery, electroporation, agrobacterium infection, or gene gun.

58.-65. (canceled)

66. A kit, wherein the kit comprises the transposase according to claim 1.

67. The nucleic acid set construct according to claim 25, wherein the exogenous nucleic acid fragment comprises a gene of a natural functional protein, an artificial chimeric gene, or a gene of a non-coding RNA.

68. The composition according to claim 39, wherein the exogenous nucleic acid fragment comprises a gene of a natural functional protein, an artificial chimeric gene, or a gene of a non-coding RNA.


SEQUENCE LISTING (303 sequences)

SEQ ID NO: 1 - protein, 563 aa, synthetic construct
MSKMSGKRKAYSVQEKLDAIERLRNGETQAKISRDIGINESTFRGWLKNADKLGTYIHNLDEEHGLQRKRARLANDADLDSAVYTWFVQEQQKGIPISGPLIAMQAEKFDRELNGEFSNFKASSGWLWRFQKRHGISQIAVSGEKRSADDDAAQSYPAKLREILQAEGYSEEQVYNADETGIYYKMLPDKTLAVRTDERKKEGYKQAKDRLTLLFCVNATGKHKLKPLCIGKSRMPRCFHHVNVNCLPFSYKNSKNSWMTREIFEQWFFTEFVPSVRKHLRAKRLEEKAVLLLDNCPAHPPAERLRTRDGKIRVEYLPKNTTSKIQPLDQGVIAIFKQHYRRNLVRQMIESELSVTDFLKKLTIKDFFYIGGDSWNLLRAETINRCWMVGLRESFGHPLLEENEESRSQSDEEFDFEGFTEEEVGRTDMEWMQELCQQLSGLGVTVLPQNIESWIRGDDEAEEFCPVAESLTDDQILQAVRPNNIPEAEELAFAVEEEEEDEVDVVPTSTATVAGLETALRWFETQDVEPIKIMQLRSLLQFAKRKRHSSKKQKQLTDFLKKN

SEQ ID NO: 2 - protein, 531 aa, synthetic construct
MASKRIKITLELKRKIIEKRENGVSVADLARTYNRSTSTICTILKNKDKIKQIDASKGVTRMSTQRLRILDDLERLLLIWINEKQLQGDTITEQIICQKAKLIFTDLRTKTPGSSTDEKEFKASRGWFEKFKKRTGIHSVMRHGEAASSDKKAAENYVSDFKKLLISEGYLPQQIFNCDETGLFWKKMPKRTYITEEEKALPGHKPMKDRLTLLFCANASGDLKIKPLLVYHSETPRAFKKCNIHKNKLNVMWKANNKAWVTRDIFVEWINEVFGPSVKKYLLEMNLPLHALLVLDNAPAHPPSLQNDLLDEFQFIKIQYLPPNTTPLLQPMDQQVISNFKKLYTKGLFEHCFEVTEETNLTLREFWKYHFHIVTSLKIIDKAWDGVTKKTLISAWKKLWPEILVESGFEAIESEPVESLDNEIVSLATDMGLTVDKDDIDELVADHSQELSTEELVELQHISQQELVEENLLDKEEKISEEQQSSSAIREMLKAWETVATHVENYHPNKAVAMRSINLFNDNAVSLLKTF

SEQ ID NO: 3 - protein, 522 aa, synthetic construct
MSSHHLKGVKKSTLTDKMRSAICEHKKENPTISQKDMQAWVQQKFDLTISQSTISNTLKRASEYLSNEMKNSDVKRHKSAKYPDLEKVLYEWFLQRQDKVNMSGEIIQGKAKELLQKMYGETNPEFSFSSGWLERFKARYGIKSYRRFGESGSVIMENIENALPGIRSKLDQFQLKDIYNMDETGLFYRLEADHSLATKQLEGRKKDKERITVVVCCNGDGSDKVPLWVIGKYANPRCFKNVNMNNLNCKYRSNKKAWMTGLLFQDFVGWFDKRMNGRKVLLIVDNCPAHPKVVEGLRNVELFFLPPNTTSKIQPCDAGIIRAFKAHYRRRFYSSILQGLEVEAPNPEKINILNAINFANMAWNFDVKTTTIANCFRHCKIRSEENNVPEPEVGELDEGIQGLNDVISNLHYRNVMDVEHLLNYPSENDAVMESPTDEEIIQSVMNSNDDENDPEPDDSSVVPNVSSKEAFQAIVTLSNYLLQHEQNIPEVVYALQKVKDGVHFGLGGKKKQSTIDAYFQKE

SEQ ID NO: 4 - protein, 496 aa, synthetic construct
MAGKRKTLSADEKRHLLESYDSLPKTSQRDAAQKLGIPQPTLSKILKDRESIIQNEGDRKRMRSGKAPMVEAALIKWIRNARERNAPLSGPLVMQKAEELAVALGVTDFKASTGWFERFKKREGIVFKKMHGEAEDADIRSREEWLENEWIEMRRDFAEEDIWNADESGIYFRALPDGTLTFKSDNKKGGKKSKERITALFICSMTGKKKDLFIIGKSQNPRCFKNVRHLPVRYAANASAWMTGYLFGEWLKDWDRKLGQERRKILLLVDNCTAHVTKDVALRNIRLEFLPKNTTSILQPCDQGIIRTAKAYFRKQMARTVLHHIDDENNKATAVEIARKISLLDAILMLANAWSDVDASTIRNCWKKGGLVIAPMEEAIVVEPPRELNAHEFEEWIDIDDNAFVTQPVSDEDIVTEIRDQFEVKPENDAEADSDDDEVIVPSLTQMKDAMKTLTNGLLYRGFNDFNLLHRLEKEVNNVVDRAMIQGTLDKFVIKQ

SEQ ID NO: 5 - protein, 429 aa, synthetic construct
MKKTSYTISFKKEVIQYMEEGNSCYKAKRHFSERDGYDYSQSMFQQWFLNREQIKICSDSMKRARGGGRKAILGSLEDMLLDEIIELRLMKIKVTRSFISDRATQLARENNIELKATGTWVTNFMTRNGLSLRRTTNFTVLSDDELIMRGVNYILFLRNTLPTVDLSKTLLMDETAIYFEDSRTQTVDLKGRKHVVMKSTGFSSMRITALISVWGNGRKAIPLVINKGKTGNSIIRVVNGIYNASQSKAWVNQDLIIAWIDMMFPRFDVSPGKCIIWDSCRAHISEKVKNHCRIRNIKMIVIPGGLTPYVQAGDIGIFKELKDKISVIINTWKNSDAVEYTRGGNPKAPNNMVVSNWVRDAWNSVSNTNILNSIKSAGFSDDINAWHISKHDIYGEMFKNTYTLRMFNSSLHRQENIETEPDIFDLADE

SEQ ID NO: 6 - protein, 441 aa, synthetic construct
MEPPTKRRKYEASFKLKVVDVAKGSNNCAAARQFDVTEKMVREWRKKEDDIRQMPKNKCAMRRGITRWSKLEDDVAEWVLQQRQDGYIVTRNKIRSHALKWAKANKEESKDFKATVGWCSRFMNRRNLVIREKTKIAQKLPKDLEHKITNFHHFIIQLRKRHKFPLSHIGNMDETPMNFDMLGNKTVDFKGVRTVNVKSTGHEKTRFTVVLGCMADGTKLKPMVIFKRKTKPKITFPPGVLVHFHEKGWMDENGVKLWIENIWNRRPGGMRKERSLLVWDMFRSHITANSKTRLARNNTDIAVIPGGLTSVLQPLDVSLNKPFKDNARAQWNEWMMNGEKSFTKNGAMRGASLDVLCEFVIKAWDNVKVESVIKSFKKCGISNAMDGSEDDLLYESEDEAEVDLPEPEWNPYDDGICDESRDVFEKLFETDGDDGDEFDGF

SEQ ID NO: 7 - protein, 436 aa, synthetic construct
MAKTRRNAYDAAFKLKAIDLAVGKGNRAAARELGLNESMIRRWKQQREELTQCKKTTKAFRGKKSRWPELENELEDWVNTQRADGRGVSTVQIRLKAKTIATAMKFEDFRGGPSWCLRFMRRKGLSIRVQTSLCQQLPPDYEEKVSNFRKFTDAKIAEHSIGPHDIINMDEVPLTFDLPLTRTVNRKGESSVTLKTTGHEKTHFTCVLSCTASGEKLPPMVIFKRTTMPKEKFPRGIVVKVNKKGWMTESLMHEWHTECYGKRPGGFFHRNKALLVLDSMRAHITDSVKEAIKRTNSIPAVIPGGTTKYLQPLDISVNRAFKVALRVQWEAWMTSGEKSFTKTGRMRRATYGQVCQWVLTAWSIVKKSTIINGFRKAGLLRVEEGSAGDLPPDESDESDNENDPISDEAILRLFNSDTEGDDFSGFSAQEEEDSDQ

SEQ ID NO: 8 - protein, 425 aa, synthetic construct
MAPVKRHAYEAQFKLQAISYAVVNGNRAAAKQFHINESMVRKWRKQENELRQVKKTKQSFRGNKARWPQLEDQLEQWIIEQRTAGRSVSTVTIRLRATKIAQDMGIEHFQGGPSWCFRFMKRRHLSIRARTTVAQQLPADYKEKLDIFRTYCSNKITDKKIQPKHITNMDEVPLTFDIPVNHTVEKKGASTVSIRTTGHEKSAFTVVLGCHGDGQKLPPMVIFKRKTLPKEKFPAGVIIKANQKGWMDEEKMNEWLREVYVKRPDGFFHTSPSLLICDSMRAHLTATVKNQVKQMNSELAVIPGGLTKELQPLDIGVNRAFKVKLRAAWEQWMTDGEHTFTKTGRQRRASYATICEWIVDAWAKVSALTVVRAFAKAGIIAEQPPGNETDSDNDEREPGVFDDETAQLFNSDTEDEDFDGFVGED

SEQ ID NO: 9 - protein, 602 aa, synthetic construct
MARKKAKTRVSYQKNDPSVMEQAVKAVQSGALSLRKAAEKFNVHKSTLHDRVTKRVTIDAKPGRKPVLSNEIEKRIIKNVTESAEKGFGISRKQLLHRAAVLCKRNKITGFKNFTPSKHWWEGLKKRHPEVALRKPEKLGTVRARMLNGCVVDKYFADLSKIVVNLNLQEKPENIWNCDETGKQLEHTPVNVIARRGSKSVVGRTGNDRSNITIMACVNATGDSMPPMLIVKGKTKKSLFGFNTAESPQNSVWTYNEKAWMNEELGEQWFREVFLAHCGPERPQLLILDGHGSHETLGLLELAAEENIHILALPPHTTHFLQPLDRTVFGPFNKAYNRVCSEFLSENPSNLINKWTFPKMFSKAWEAAITKENIQSGFRACGIYPLNPHAIPTSAYLPSSAYETSMPVAETPAALGRPPSPSAADNENIPSINMPIPPETESQPMHNATSTMPLIDSVSHDQPTPNIISLEQLPLQTTSLPTTPSSQNFEVDDPSYLLQLINSGEIQVVATADNEGVATLPVDTVWNEHIDAIFSPSVNAPRTAVESKKKSRAFTSHRLLTSVEVVNEKRAVAEKKKKIEEEKQNRKLKREQNKLKREKKTA

SEQ ID NO: 10 - protein, 666 aa, synthetic construct
MPFLFKKKNKRREIPKDVILRALEEVSKGGKIKTTAIKYDIPRSNLQRYIKQGGAVKDTSCKYISSQIFTKDEEEKISEYFVTLFKLNHGMSKLKARELVYEYATAINKKIPDNWTEKKLASKDWLRGFFKRQPQLSIRTPEATSLSRATSFNKKNVGDFFENLKTVYERHRFGPESIYNIDETGLSTVQRTQKVIALRGTKQVGQVTSAERGTLVTVCCGINALGNSIPPFFIFPRVIFKTYMLNDAPVGSDGAAHPSGWMTASSFLKYIHHFKKYAKPTPSSPVLLLLDNHESHISVPVLDFCIESGIVLITFPPHCSHKLQPLDLTVYGPLKTYYNTAVTDWMVSNPGKTVTIYEIPKLAAKAIPLAFKLQNIQTGFEKSGIWPFNSNIFSDEDFVCSSITDRYLPGENNVATEKSISDQPIMEQPSTEENLRLEDRTSSNVEVTGPSQLESLSKSQTSGTVNVTPDLVRPYPKAGPRKLGGMKRKKGKTRILTDTPEKRLIEIEEEKKQRKNKIKELNKNRKCSKNEKNRKISSDDEIKNVSVSDEAFVEIDSSKEDFDVDDVIVLDRNFQVNNFVLVKFDTKKTVYHYVGIVEEVNHSMATVKFMRASKIRNTFMFPDIEDVASVPLEDIKTKLPTPSTWIKRGSLKYKFDKPLWVIPNLR

SEQ ID NO: 11 - protein, 339 aa, synthetic construct
MKPKEHTRQVRDKVVEKFKAGLGYKKISQALNISRSTVQSIIRKWKEYGTTANLPRHGRPPKLTGRARRALIREAAKRPMVTLEELQRSTAQVGESVHRTTISRALHKSGLYGRVARRKPLLKKSHKKSRLQFATSHVGDTANMWRKVLWSDETKIEVFGLNAKRYVWRKTNTAHHPEHTIPTVKHGGGSIMLWGCFSSAGTGKLVRVDGKMDGAKYRAILEENLLESAKDLRLGRRFTFQQDNDPKHTARATMEWFRSKHIHVLEWPSQSPDLNPIENLWQDLKIAVHRRSPSNLTELELFCKEEWAKISVSRCAKLVETYPKRLAAVIAAKGGSTKY

SEQ ID NO: 12 - protein, 340 aa, synthetic construct
MPRSKEIQKQMRKKVIEIYQSGKGYKAISKALGLQRTTVRAIIHKWQKHGTVENLPRSGRPTKITPRAQRRLIQEVTKDPTTTSKELQASLASVKVSVHDSTIRKRLGKNGLHGRVPRRKPLLSKKNIKARLSFARKHLDDPQDFWENTLWTDETKVELFGRCVSHYVWRKSNTAFQKKNIIPTVKYGGGSVMVWGCFAASGPGRLAVINGTMNSAVYQKILKENVRPSVRDLKLKRTWVLQQDNDPKHTSKSTSEWLKKNKMKTLEWPSQSPDLNPIEMLWHDLKKAVHARKPSNVAELQQFCIDEWTKIPPQRCNRLIASYRKRLIAVVAAKGGPTSY

SEQ ID NO: 13 - protein, 340 aa, synthetic construct
MPRSKEIQEQMRNKVIDIYQSGKGYKAISKALGLQRTTVRAIIHKWRKFGTVVNLPRSGRPTKITPRAQRRLIQEVTKEPTTTSKELQASLASVKVSVHDSTIRKRLGKNGIHGRVPRRKPLLTKKNIKARLTFGKKHLDDPQDFWENILWTDETKVELFGRCASRYIWRKSNTAFQKKNIIPTVKYGGGSVMVWGCFAASGPGRLAVIDGTMNSALYQKILKENVRPSVRDLKLKRTWVLQQDNDPKHTSKSTSEWLKKNKMKVLEWPSQSPDLNPIEMLWRDLKKAVHARKPSNVAELKQFCKEEWAKIPPQRCERLIASYRKRLIAVVAAEGGTTSY

SEQ ID NO: 14 - protein, 340 aa, synthetic construct
MPRPKEIQEQLRKKVIEIYQSGKGYKAISKALGIQRTTVRAIIHKWRRHGTVVNLPRSGRPPKITPRAQRRLIQEVTKDPTTTSKELQASLASVKVSVHASTIRKRLGKNGLHGRVPRRKPLLSKKNIKARLNFSTTHLDDPQDFWDNILWTDETKVELFGRCVSKYIWRRRNTAFHKKNIIPTVKYGGGSVMVWGCFAASGPGRLAVIKGTMNSAVYQEILKENVRPSVRVLKLKRTWVLQQDNDPKHTSKSTTEWLKKNKMKTLEWPSQSPDLNPIEMLWYDLKKAVHARKPSNVTELGQFCKDEWAKIPPGRCKSLIARYRKRLVAVVAAKGGPTSY

SEQ ID NO: 15 - protein, 340 aa, synthetic construct
MPRTKEISEDLRLRIVDLHKAGKGYKSISKSLDVHQSTVRQIVYKWRKFSTVATLPRSGRPAKMTARAQRRMLNEVKKNPRVSAKDLQKSLEHANISVDESTIRKTLNKNGVHGRTPRKKPLLSKKNIAARLKFAKVHLDVPQRYWQNILWTDETTVELFGRNTQHYVWRKKGTAHQHQNLIPTVKYGGGSIMVWGCFAASGPGQLAIIDGKMNSQVYQDILQENVRLSVRQLKLNRSWVMQQDNDPKHRSKSTTEWLQQKKIRLLEWPSQSPDLNPIEMLWHDLKRAVHTRHPKNIAELKQFCKEEWSKIPPDRCAGLIRNYRKRLVEVIAAKGGSTSY

SEQ ID NO: 16 - protein, 340 aa, synthetic construct
MGKTKELSQDLRDRIVDLHKSGTGYKNISKMLGIKVTTIGAIVRKFKKYNLTINRPRSGAPQKISPRGVAMIMRTVRNRPATTQRELVNDLKAAGTTVTRKTIGNTLRRNGLKSCSARKVPLLKKAHVKARLKYANDHLKDVQSDWEKVLWSDETKIELFGLNSTRHVWRKKNAAYDPRNTVPTVKHGGGSIMFWGCFSAKGTGLLHRISGKMDGAMYCAVLGDNLLPSARKLKMGRGWVFQHDNDPKHTAKATKEWLKKNHIKVMEWPSQSPDLNPIENLWRELKVRVAKRQPTNLNDLERICKEEWAKIPPDMCANLVVKYNKRLSAVLANKGCATKY

SEQ ID NO: 17 - protein, 340 aa, synthetic construct
MAKTRELCKDIRDKIVDLHKAGMGYRTIGKQLGEKATTVGAIIRKWKKFKMTVNHPRSGAPCKISPRGASMIMRKVRDQPRTTRQDLVNDLKRAGTTVSKKTISNTLRRHGLKSCSARKVPLLKPAHVQARLKFANDHLDDPEEEWEKVMWSDETKIELFGLNSTRRVWRKKKDEYNPKNTIPTVKHGGGNIILWGCFSAKGTGRLHRIEGRMDGAMYREILANNLLPSVRALKMGRGWVFQHDNDPKHTARATKEWLRKKHLKVLEWPSQSPDLNPIENLWRELKVRIAQRQPRNLKDLEKVCMEEWAKIPAAVCANLVKNYRKRMISVIANKGFCTKY

SEQ ID NO: 18 - protein, 340 aa, synthetic construct
MAKTKELSKDTRNKIVDLHQAGKTESAIGKQLGLKKSTVGAIIRKWKTYKTTDNLPRSGAPRKISPRGVKMITRTVSKNPRTTRGDLVNDLQRAGTKVTKPTISNTLRRQGLKSCSARRVPLLKPVHVQARLKFAREHLDDPEEDWENVIWSDETKIELFGKNSTRRVWRTKNAELHPKNTIPTVKHGGGNIMLWGCFSAKGPGRLIRVKERMNGAMYREILSENLLPSARALKMKRGWVFQHDNDPKHTARATKEWLRKKHFKVLEWPSQSPDLNPIENLWRELKVRVAQRQPQNITALEEICMEEWAKIPATVCENLVKTYRKRLTCVIANKGYITKY

SEQ ID NO: 19 - protein, 340 aa, synthetic construct
MPKVKEIPEATRKRIIQLRSEGLTYRVIAERVNVPFSTVGAIVRKHQVTGAVSNLPRAGAPRKIMGRPRRYMLRKVTNDPMTTRADLQSELASQGLHVSKKTIVRELNRTGIHSYTPRKTPLLTAKHVKSRLTFAKDHLDKGNEFWEGVIWSDETKIELFGRNEARHVWRKRGTAYDPKNTIPTVKHGGGSIMVWGCFTAHGVGDLHIIDGKMNSFMYREILEKKMLPSAKKFYGRRRWTFQQDNDPKHTANLTKEWFQKKKINVLPWPSQSPDLNPIENLWRLFKSGVHKRCPKNLTELKVICMEEWKKIPSSECEKLISSYNNRLHAVIANKGHATKY

SEQ ID NO: 20 - protein, 340 aa, synthetic construct
MGKSKELSMDLKKRIIDLNKSGKSLGAISKQLQVPRATVQTIVCKYKVHGTVLSLPRSGRKRKLSPAAERKLVRRVKSQPRTTKKQICQELEAAGTQVSVSTVKRVLHLHGLRGCRARRKPLLQKRHLKARLKFAADHMDKDKTFWRKVLWSDETKIELFGHNAQQYVWRRKGEAFNPKNTTPTVKHGGGSIMLWGCFAANGTGALQRVNGIMKKEDYLQILQDNLKSSARRLGLGRSWVFQQDNDPKHTSKVVIEWLNQARIRVLEWPSQSPDLNPIENMWTMLKKQVHVRKPSNLTELHQFCQEEWSKIQREACQKLVDGYQKRLIEVKMAKGHVTKY

SEQ ID NO: 21 - protein, 340 aa, synthetic construct
MGASKQLSNDLKTKIVQHYGLEEGYKKLSQRCKLSVSTVRNIVRKWKTTGTVLVKARSGRPSKISERQRRRMVRTVKNSPQTTSKDLQHHLAADGVTVHRSTIQRTLHKEKLYGRVMRKKPFLHTRHKQSRLRYANAHLDKPASFWNKVLWTDETKIELFGHNKGRYAWRQKNTAFQEKHLLPTVKFGGGSIMLWGCVASAGTGNLVKVEGRMDSTQYQQILGNNVEESVTKLKLRRGWIFQQDNDPKHCSKSTRAFMQRNKYNVLEWPSQSPDLNIIENLWDDLKRAVHARQPSNLTELEMFCKEEWSKIPSSRIQTLIRGYRKRLEAVILAKGGSTKY

SEQ ID NO: 22 - protein, 340 aa, synthetic construct
MAPHGKELSEDLKKRIVALHKDGLGYKKIAKTLKLSCSTVAKTIQRFNRTGSTQNRPRHGRPKKLSARAQRHIQRLSLGNRRMSAASIAAEVEGVGGQPVSAQTIRRTLHQIGLHGCRPRRKPLLKMMHKKARKQFAEDKQTKDMDYWNHVLWSDETKINLFGSDGVKRVWRQPGEEYKDKCVLPTVKHGGGSVMVWGCMSAAGTGELQFIEGTMNANMYCDILKQSMIPSLRRLGRRAVFQHDNDPKHTSKTTTALLKKLRVKVMDWPSMSPDLNPIEHLWGILKRKVEECKVSNIHQLRDVVMEEWKRTPVATCEALVNSMPKRVKAVLENDGGHTKY

SEQ ID NO: 23 - protein, 336 aa, synthetic construct
MSRKQISLDIRNLVIRDRQSGDSFRIIANKYSISVGAVQHIWKKYKTHGIVKNYSGKGRKRATTFRDDTRIVRMVKQNPKLSSRNIVESMNLNVSSRTVRRRLRESGLKNVFASKRPFINKINKKKRFTFAKKYANMPLSFWKKVLWSDESKFELFGQKRRSRVWRKPGEALKEVNIQKTVKHGGGNIMVWGCFAWSGIGNLAQIEGIMTAEGYIDILCENLEESLLKLDLENNFIFQQDNDPKHKAKKTTAFFKSNKVKLLEWPPQSPDLNPIENLWAYLDGNVDKTNVTHKTSYYAALQKAWEEIDPEYLKKLVESMPRRLEAVLKAKGGHTKY

SEQ ID NO: 24 - protein, 346 aa, synthetic construct
MGQPRKEIPIQLRDLIIQDWKGESSVKLSMGKISEKFKIPKSTVQRTIQRYRETGGLQTKKRTGRKKILTPREEIKLVRKVQRNPFLSAPKLAAQIVQESGKTLHPSTVRKIINKNGLHGRIPRKKPYLSKVNRKKRLAFARKYVLKPPSFWSSILWSDETKVNIFGSDGHQKVWRRPKEALKNSNLRPTVKHGGGSVMVWGCFGAKGVGNLEFIEGNMTGVSYTGILNRNLFESGRKVGLGRRFTFQQDNDPKHTSKVAKEFFKINKVKVLDWCPQSPDLNPIEHLWDVTKREVGKTNVKNKSELKTRIREVWDNLDPEVIRNLVSSMPKRLEQVISANGGHTKY

SEQ ID NO: 25 - protein, 340 aa, synthetic construct
MAPHVKVTPELREEIVKLRSSGMTLSAISREVNRSKSVVSRALKLYDEKKQLESPVSAGRPRKTTVRVDRIMKRYAQKDPFETAAGISRKLKVNNNIEVSRYTVSRRLNACGLKARSPATKPLISKKNKKARLEYAERHIVWRDEDWERVHFSDESKFNLVGSDGRRYVRRATGDRLNPKCVKTSVKHGGGSVMVWGMFSSEGVGPLVAIKETVNANVYLNLVQQYVIPSLRSSPNQPAIFMQDNAPCHTAKKVKEYFAEENIEVMKWPAQSPDLNPIENLWKIIGDNVRERQPSTVKDLWTKIEEEWKKITPEFCKKLVMSCNRRCAKVIENKGFSTPY

SEQ ID NO: 26 - protein, 340 aa, synthetic construct
MATREKVDKSLRMKIVNLHKQNLSIRAIAEQVNRSKSIVGRIVKDYEDLGRVSSVRKRGRPRKTSARLDRIIQRTALKDRFATASSISRQMNDTNDETISRFTVSRRLNEIGLRARRPQKKPLISKKNKVARLKFANKHVNSPQEWWSKVFFSDESKFNLFGNDGNNYVRRQTGEKYDPRCTKKTVKFGGGSVMVFGMFSAKGTTPLVRLNTRVNAAVYKNLLADHVLPILNNSDTDGPIFMQDNAPCHKAKSVLRFLDEEGVETLDWPAQSPDLNPIENLWKVLGEKVMARNPTNTEDLWTKLQEEWGRIDIERCRNLIESCPRRCVEVIKHKGSFTKY

SEQ ID NO: 27 - protein, 331 aa, synthetic construct
MGRGKHCTPERRELIKNLISSGKTYKEVQKIIGCGASMIRNAIKFSPKTETRGRPQVLADKLATRVARYSKKHPFASCKEIKDELNLDASIPTISRLLKKQNLKAHSPRKIPFHTKKHIAARMKFAKSHVNWSVEKWRNILWSDESKIVLYGGKGSRQYVRRPPNTEFNAKYCIKTIKHGGHNIMIWACFSYYGVGPIYWIKNIMDGPGYVQIMQETMLPYASEEMPLLWVFQQDNDPKHTSKVAKKWFAENKIAVLEWPAQSPDLNPIENLWTDVKKAVAQRHPTNVSELWEIVKASWTSISPERCQKLVDSMPRRCAAVIANKGGATKY

SEQ ID NO: 28 - protein, 337 aa, synthetic construct
MPTGTPIGKELKERVIDAFLNNEKQADIARRFQLPRYSVSKIIIRYNERGHLNNNPKSGRPRKTTINMDRRIKRISENDPWKSASKIIAEIPEVGVSTRTIQRRLVAAKLFSRRPAKKPLISERNRRARLQFAREHLNWTVQDWRKVLFSDETRYKLFNSDGMKRVRRPINTRFNPKYVTPTVKHGHGSVFLWGCFSWNGVGPLSFIEGNMDRFIYRDILQDVMLPYAEWEMPLRFIFQQDNDPKHTSALVSEWFQNNNIHVLQWPAQSPDLNPIENLWEEVERRIRIQRFPNKQSLIDKIKEVWSNLDENVIQKLISSMPRRCQKIILNNGYAINY

SEQ ID NO: 29 - protein, 340 aa, synthetic construct
MPKQKEYSDDIKSAVIAALKNEKSQAQVARDFNLSRQIISVWNRKEQSTGSVSNKQRSGRPPKLTDHVSRIIRRMSVANPRLTAADIKRDLNENHSLDISLTTVKQCLRNANLFGRRPSKKPFISARNRKARLEFAQKYKNWTSQEWARVLWSDESKFNMFSSDGIRYIRRPPNKRDDPKYQVPTVKHGGGHVMVWGCFSRSGVGPLVEVKDIMDRFRYCEILQNHMLPYAKRNLARNWIFQQDNDPKHTSAHVKEFFTSKKIKVLEWPSQSPDLNPIEHLWEELDRRIRKRKFSRKQDLMACLAEEWKEIPLTVITKLIDSMPARCAAVIKSKGFATKY

SEQ ID NO: 30 - protein, 338 aa, synthetic construct
MVTCKETRAVIIALHKKGFTGKDIAASKIAPKSTIYRIIKNFKESGSIVVKKASGRPRKSSKRQDRLLKLIQLRDRGTTSTELAQEWQQAGVSASARTVRRRLLEDGLVSRRAAKKPLLSRKNIRDRLIFCKRYRDWTAEDWGKVIFSDESPFRLFGASGKKLVRRRQGERYHQSCVMPTVKHPETIHVWGCFSAKGVGSLTILPKNTAMNKEWYQHILREQLLPTIQEQFGDEQCLFQHDGAPCHKAKVITKWLGEQNIDILGPWPGNSPDLNPIENLWSILKRRVDKQKPTNSDKLQALIMQEWAAISQDVAQKLIDSMPGRIAEVLKKKGQHCKY

SEQ ID NO: 31 - protein, 334 aa, synthetic construct
MRKLSEDIENNVVSLTENGCTSREISKRLGISQSAVIAVQKRRNVTPKAQVAGRKKLLKESDARLMMSEMRKNKLLTPKGASLAINKNVSEWTARRALRDIGYIASVKKIKPALSVKNVKARLKFARAHKNWTVDDWKRVVWSDESKFNRFQSDGKQYCWRRPGDRIQRHHVKQTVKHGGGGIMVWGCFTWWHVGPLQLIEGIMRKEDYLQILQTNLPNFIEKCAYPEKEIVFQQDGDPKHTAKIVKEWIEKQPFKLMEWPAQSPDLNPIENLWSIVKRRLGQYEAAPSNMGILWERVEAEWRRIPKDIIENLVESMPKRVQEVISNKGLWTKY

SEQ ID NO: 32 - protein, 334 aa, synthetic construct
MKKLSADTENSIVAMTKSGFSTRKIAEKLKISQSVVVKVKKRQNVASENVLNGRPRLLTDSDARLIMSNMRKDKLLTPANASTELNKPVSRWTARRALARIGYVASVKKNKPALSEKNVKARLQFAKTHKNWTIEDWKRVIWSDESKFNRFQSDGKQYYWHRPGDKLQRHHVKQTVKHGGGNVMAWGCFTWWHVGPLHKIDGIMRKEDYLNILKTHLHNFLDKCAYPEGEITFQQDGDPKHTAKIVKEWLAKQNFKVMEWPAQSPDLNPIENLWSIVKRRLGQYQSAPSNLDDLWRRVEIEWNQIPKEIIENLVNSMPKRIKNVIDNKGLWTKY

SEQ ID NO: 33 - protein, 335 aa, synthetic construct
MPRLDIVSRNIAIGRLEAGESQYVVAQHYNVHRSTISRLWQRYLQTGTSNDRPRTGRPRITTAQQDRYIRILHLRNRTVTATQTASSIPGLRRISAQTVRNRLREVGLRARRPYFGPILGRQHRRARVGWCNTVRNWTLRNWRRIWFSDESRFMLQHRDGRVRVYRRKNERFANNCVVEVDRFGGGSVMMWGAISYNQRTPLVLVPGNLTAQRYRDEILQPHLLPVINTQREVFQHDNARPHTARATVDFLADHNVTVLPWPSRSPDLNPIEHLWDQLDKRVRMRQPAPQTLLQLQQALHEEWQRIPQVQIQRLIQSMRRRVTTVLQANGGHTRY

SEQ ID NO: 34 - protein, 339 aa, synthetic construct
MPRVPAHLRERALGMLQGGMRTADVARAINCNVRTVRRLRQRYRETGRTADRPRSGRPRVTTPAQDRYIRTSHLRDRYRMATTTARVTPGTHNPSISAQTVRNRLREAGLRACRPVVRQVLTRHHRQQRRLWAQTHRRWTRQDWQKVLFTDESRFCLTRGDGRIRVYRRRNERYTEACTLERDRFGGGGSVMVWGGVSQHHRTELVVIAGNLNAVRYREDILLPHVVPFLQAHPDMTLQHDNATSHTARSVRDFLQDRNVSVLPWPAKSPDLNPIEHVWDLLDRRVRARAIPPRNVRELAGALVEEWGNISQQELANLVQSMRRRCTAVLNAAGGHTRY

SEQ ID NO: 35 - protein, 341 aa, synthetic construct
MARRRLSDATRWQIIGMHNAGMSYSAIGRNLGYNHTVISRLVNKHLQTGAVKDRPRSGRPPVTSQREDRDLQRLVRREPFATSTRLKSLWLPNRRFSLRTLRNRLRSTGMKARRVIKRPLLTDRHKAARLAWCIARQNWNMRSWRRVHWSDESRFLLHVTDGRTRVWRHRNTAYTPRNIRPTVPYGGGSVMIWGCISHDCKLDLVTIRGNLTGQQYIRDVLQPIVVPHFDNHTLASRPQFMDDNARPHRSRAVNDFMRTSAITTLPWPAMSPDLNPIEHVWDFIGRRVRAREPPVQTLRQLEAALHQEWRQLTLEEVRRFTGNMRNRVHCVIQARGGYTRY

SEQ ID NO: 36 - protein, 332 aa, synthetic construct
MGRQCGLSEKEKGQISALNEEGYTISEISRKVSRSRKVVTNFLKNPEEYGKKKSPGRPSKLSPTARRRLLREASRTGEGSKSLQTSLQLNVTPRRVRQILGSSKNFSYKKRKTAPALTKAHKAKREEWVREKVQWNHENWSNVIFSDEKKFNLDGPDGSQYYWHDLRKEDKIFSKRPFGGGSLMIWGAFSIKGKANLVMMEGRQNAQKYTEVLETSLLPFQTDKHQQEAVFQQDNAAIHTARLTTNWLNAHNIATLAWPAKSPDLNPIENLWGILSRHVYANGRQFQDKESLWQTINECWEAIPEEMLANLINSMNNRCVQVLQSKGNACKY

SEQ ID NO: 37 - protein, 342 aa, synthetic construct
MSELSAKLRYILQFYFDKGANAAQAREEICAVYGQDTLSKATAKRWFSRFRSGNFDVQDAPRSGRPITEKVDDILAKVEQDRHVSSHDIANDLNIHHQTVLNHLEKAGYKKKLDVWVPHDLTQKNLLDRISICESLLKRNEIEPFLKRLITSDEKWITYDNNVRKRSWSKRGEAPQSVAKPTLTPRKVMLCVWWDWKGIVHHELLPPGRTIDSDLYCRQLARLHLAIQKKRPELVNRKGVVYHADNARPHTSLTTRQKLRELGWEVLMHPPYSPDLAPSDYHLFRSLQNSLNGVKLVSKEACENHVSQFFDQKPQKFYSDGIMVLPEKWQKVIEQNGAYVVS

SEQ ID NO: 38 - protein, 370 aa, synthetic construct
MPSPKRSPAEKRSPAEKRRVLEAYRAGRADWLKVAANNGISRSMAYRIADTGRVEDLPRGGARRSVVKVTPEVKDHLEAYLNDNCTYTMETMQRMLIVDLGLTISTSTISRHLLGMLYTVKQTRVEPTTCNNETNKTKRQEFAIRLKEHQRVGNCIVYFDETNYNLYCKRGRGRAKKGERATVVLPPSKGANLQVQCAVNSAMGVVLYRMRRGSIRMEQNAAFIDDIYRAVKESQVFRENFSGKKIVVVLDNAPAHRQTEERVTPHEDLELLRLAPYSPMCNPIEGCFSVLKARIKNHLALDRGEICDRSNMQDSEGNILTVKERTMRFLERAALASIKYITPTIVTNMELHCRDAVNAAADMEDMVYGK

SEQ ID NO: 39 - protein, 337 aa, synthetic construct
MNAERRQKNKISNEVRALIIRGMNDGSSSVQIGRQFNVLPNTVRKIYKTYKITNEVNKRSAGHRPKKFTQEEQEEICNLMDEDCTLTLKQVKAKLLEQNPQLEVSLSTIDRATKAFHYSVKRVSFVPERRNTPDTIQSRFEYAVQYNRIMVHREKLFFIDEFGIQVNSRRAYGRSPKNTRANKVKAQLRGRNYSIAAAMCINSLYLFTIQDRPYNEEHFGEFLTSLIDHLAADHIQGAYFVMDNVGFHRTDEIQQLIRAHGHVPIFLPPYSPFLNPIEELFHQWKTIIKRSQPTDEEQLYVQIHNASEAITQEHCQNYVRHMEIYLGDCLNRIEILN

SEQ ID NO: 40 - protein, 450 aa, synthetic construct
MPNSQGPNMAPLTRRKKYEASFKLKVVNFAMEHNNCAAARHYGVTEKMVRDWKANEKALKNMPRGKCALRRGTPHWPELEKHVADMVNEHRQNGYVVTRNKIRLFALQWAKSNPDHSNRFKATVSWCTRFLERHNLVLRQKTKIAQKLPADLDAKLNNFHRYVIKQRNKHGYALNSIGNMDETSMNFDMVGNKTVHLKGEKTILIKTTGHEKSSFTVVLACTADGAKLRPMVIFKRKTMPKLKFPVGCFVHVNEKGWMDEEGVKLWLDNVWSRRPGGLIKKRSLLVWDMFRAHLTPSTKERLARLNTDSAVIPAGLTSLVQPLDVCLNKPFKDRIREQWNEWMVSGEKSFTKGGNMRAPQLDVLCKFVIKAWNDIDAKTVIKSFKKCGISNSLDGMEDDYLWQNEEEVEAETTPSDTEFDPYDDCLTNVEQDVIDVLMISNDEQEDFKGF

SEQ ID NO: 41 - protein, 426 aa, synthetic construct
MAPAKRHAYEADFKLRAISYAEENGNRAAARQFSINESMVRKWRKQKDDLKQVKKTKRSFRGNKARWPQLEDKIEQWVIEQRTAGRSVSTVSIRLKATAVARDMQINDFRGGHSWCFRFMKRHNLSIRTRTTVSQQLPNDYKEKLAIFRTYCQNKITEKKIRPEHITNMDEVPLTFDIPVNRTVEKTGTSTVSIRTTGNEKSSFTVVLGCQANGQKLPPMVIFKRKTLPKEKFPAGVIVKANSKGWMDEEKMSEWLREVYVKRPDGFFHKSPSLLIYDSMRAHLTEAVKTQVKQTNSELAVIPGGLTKELQPLDIGINRSFKVKLRAAWEHWMTEGEHTFTKTGRQRRASYATICQWIVAAWAKVSVSSVVRAFTKAGIITEQLSNSNETDSDNDERDPGMLDAEIAQLFNSDTEGEEFDGFVEVE

SEQ ID NO: 42 - protein, 432 aa, synthetic construct
METTRRSSYDASFKLKAIDLAIQEGNRAAARKLSVNESMVRRWRRQREELMQCQKSRKAFRGHKSRWPELENVLEEWVNTLRAGGRGVSTVQIRLKAKTIAREMNIKDFKGGPSWCFRFMKRKNLSVRARTTLCQQLPPDYQEKVENFHKFVEKKIQENSIGPDDIINMDEVPLTFDLPLTRTVNKTGASSVLLKTTGHERTHFTCVLACTASGQKLQPMVIFKRITMPKEQLPKGIVVKVNTKGWMIEGLMKEWLTECYGKRPGGFFHRKKALLVLDSMRAHITDSVKAAIKKTNSIPAVIPGGTTKFLQPLDISVNRAFKAALRVEWETWMTSGEKSFTKTGRMRKASFSDVCQWILTAWSAVKESTITNGFRKAGLLREESGAPSEPTSPEDDSDTESETDGDRCDEALLSLFNSDTEEDDFMGFSEDD

SEQ ID NO: 43 - protein, 421 aa, synthetic construct
MEKKRRHSYTSSYKLQVVKYAEEHGNRQAERHFGPPPTEKMIRTWRGQKEALQNSRKEKRAERGKNAKWPELEYSVLKWVQENRENGIAISTKMIRTYAINLACERNIEDFKGGESWCYRFMKRSGLCMRTKTKISQKMPKEYEDKIISFHKYVIQQRKNFHFEMGQIANMDETPLTFDVPSNRTVAGKGQKSITIKTSGHEKTHYTVVLACCADGSKLPPMIIFKRKTLPKEKIPTGLIVHVHEKGWMDESGMKVWIEKVWGRRNGALLKKKAFLVLDQFRAHLKESIKQRFQECNTELAVIPGGLTSQLQPLDVSLNKPFKVFMREEWNKWMMDASVHQLTPKGALKRPSISQVCEWVKTSWSKVNEQTIVKSFLKCGISNALDGSEDHFLYEDDDEEEEERKEEEEESDDSDMDFAGF

SEQ ID NO: 44 - protein, 423 aa, synthetic construct
MAPKRKSYSADYKLQVVKYAAENGNRAAERKFGVSEKLVRDWRKAEVTLTEMKKTKKANRGLKARWPQLEERVRTWVLEQRAAGRGLSTVHLRLHAQVFAKEMNINDFAGGPSWCYRFMQRNRLSIRARTTMSQKLPADFQVKVDSFREFIEKHVSEHNVTPDHIINMDEVPLTFDIPMGRSVAEKGQKSVNIVTTGHEKSHFTVVLACCGDGSKLPPMVIFKRKTMPKIQSPSVAVAVNEKGWMDQEMMNLWLTKCYTKRPDGFFRARKALLVMDSMRTHITPQFKNKLKIFNSIPAIIPGGLTKILQPLDISVNRSFKAVLRNLWEQWMMDGEHSFTATGRMRRATFLEVIGWIDKAWASVTTETILSGFRKAGIIGTATDDESDVSDVEEEAPHLLPPELAELFKSDTEDEDFIGFSYLE

SEQ ID NO: 45 - protein, 534 aa, synthetic construct
MAEKRKRVVLGLDKKLEIIKRLKKGETATSLAQIYGVARTTVNDLKRDADKIEEHVSKMQNTDGDVKTRKTMRPAKFEQLDSAVYQWFVQARSQGIPLSGPIIVTKAMDMNKKLDGDPTFKASMGWLEKFKFRHGIRQLDITGEKLSSDSAVVEGFRAEFTAKMYDLNLSREQVYNCDETGLNWKALPQKTLASFSEKSAPGFKIQKDRITAMVCANATGNHRLPLAVIGKSKKPRAFKNLNLNALPVNYYAQKNAWMDQTIFFDWFHKVFVPQVRAHLAEKKLPQEALLLMDNAPSHPYNELQSDDGKIKCFFLPANTTPLIQPMDQGVIENMKRRYRKAFIESLLSSDDATTVKEFWRTYNIKDAIFNVANAWSDLTATNLKNAWNKLWPEPSTEESAEHEPVSVDEIVQLCNNGLIDETLSTQEVTDWLNSDNQDGGFQILSDDEIVSNVLQLDDGGDHEDVEEAEPALDNNPGITHSEAESMLTQCIDWFERQEEATGTQVLLLRKIRNMAAQKARRGKKQKKMTDYFKN

SEQ ID NO: 46 - protein, 541 aa, synthetic construct
MPPVKKKERKSVTLELKMKVLRRMENGGKATIVGRDFGLSESTVRTIFKNRAKIKVSSESSTPVSATKLSRSRSSILERMEKLLSVWIEDLNKKNTPLSQDLICTKAKSLYEMLKTEEGEGSSTPTFAASRGWFHRFKQRSNLHNIHITGEAASADIQAAAEFPDKFQEFVGREKIPACLIFNVDETGLYWKKMPSRTFISKEERQASGFKAAKDRLTLLLGGNATGDFKLKPLLVYHSETPRAMRGTDKTSLPVVWRSNRKAWVTREIFQEWYSNSFCPSVKSYCDRNGLEPKVILLLDNAPGHPTSLDTPRDLKVYVKFLPPNTTSILQPMDQGAIANFKAYYHRRTFSQLIHETDGEGRQSMREWWKSYNIMKAIHNIEEAWKEVKKSCMNGVWRNLWPDVVEAPRRIVQMQRQVLEDIAAMIEGSELEDVAVEDLVELVATHDDPLSNEDLIELQNNPIYDEEEQVEEKVLSSGGIQNALSKIYSGLDILQSIDNNDDRVLTVCQTIKNSVGCYERILQERKKNATQKTIDVFFRKK

SEQ ID NO: 47 - protein, 543 aa, synthetic construct
MSCNKMEKEKRKRKVVTIAQKLEAIKRLDKGHLLKKVAADYGVGVSSVSEWRKNREKIEQWSSVLASPNVKRKSMKSGEYNKIDEALYLWFTQQRDKGIPLSGPILQEKAISISEDLPVLEAPDTFTASDGWLGRWKKRHGIRNLKICGEKLSADKTAADCFKQNFRRIVEENGYVEDQIYNCDETGLNYKMLPTSTLASKTEAAAPGYKKSKERVTILACANYSGRHKLPLMIIGKSAKPRAFKHVAPNALPVYYKNQKSAWMNAALFKNWFFEQFVPKTEKFLKKKGLPRKALLLLDNAPSHPDVDELKSGDIKVMYLPPNVTAILQPMDQGVLENLKRNYRRGLLQEILQEVDENTNLVSCLKKINLKDVVYWSADAWNCVKSTTLRKSWSKIIEQDENDDDENETHVEDQPAEESLHELIRKIEGCENVTLEEVESWVTGDELEEELTNNELIEILDSQQEKREEIDENLEEMEDEAPRISHSEGMKALEKSLAYVEQQPNATASDVLLIKRWRDIAARNRGNMLKQTLINSFFISNKY

SEQ ID NO: 48 - protein, 499 aa, synthetic construct
MATKRKSLCIDEKVLLIRAIETGEKISDVGKRFGFSRSTVSTIWKNKEKILHAAGEGKSSKKLKKPIYEDLDKAVLSWFHRQRQNNMPISGPLVKAKAENFAAELGLTSFKASEGWLGKFKQRHHINYGKISGEARSVDTNVTHDWINRVWSKFIEKYAPSDIFNADEAGIFYKLTPDKTLKFKGEKCVGGKLSKERITVLVAANMDGTEKKKLLIIGKSKNPRCFKNIKKLPVTYKANKSAWMTSQLFEEEVRKWDAELKGRKILLLVDNCPAHPFISNLQNIELAFLPPNTTSVLQPMDQSVIKSLKGHYRRKLLMELVESEGKTSVNMLHAVNFLSKAWEEVTPTTIQHSFRHAGLCTNSTSDEVETESEFESDDDLPLTEWIQQFNMAGNTVNLQTYIEVDDRLLTTASLTDEEILDSVRKTEDQEQGDEEDETDEPELPPSIKEALEAAKLLEKYFLYNQDASVLLDINKITKKIQQNYWCSKRRQTKITDYTQ

SEQ ID NO: 49 - protein, 519 aa, synthetic construct
MAGKKSKTALTVEFKRKVLKHVEENPLKKKITIAKEFNIPSTTLATIIKNRDKYDEASGLSKFCKRAKTGEFQEVDKATLKWLKQCRDKNLSIGGPILQEKAQQFAVQLGHPDFRASNGWLEKFKKRNDIVFKNVCGECASVDSTVCDDWKEKLADITQGYHPDDIYNADETGLFFKCLPDKTMTFKGDPCNGGKHSKDRLTVLLGVNASGKNKLKPLVIGKSQKPTCFKNVNSLPTLYKANSKAWMNGDEFSAWLKTVNTEMKKKKKKILLFIDNCSAHGTVPNLSNISVQFLPPNTTSKLQPLDQGIIKNFKVNYRKEVVRQFLTDMDQDVPTKINVLNAMWMINKAWSNVTDKTVFNCFAKCGFRVTDLEDEDDDQPRTPAPQNAEVGPDQNWNKVTERLGVQEMEFEEFVDFDDNVAVCGELTDEDILDSVQPSNHKEDDAAEDDEEDAAAESDRPELSTKDAREAIDVLKCFFLKKNALSDSVVKAIIDLDSTLDMVTLHASKQSKIDDFFKIS

SEQ ID NO: 50 - protein, 513 aa, synthetic construct
MSQKRKQINIEEKSRIISKLEKGIPNKDLAKEYGVSHSTISTIWKEREKIQILYDKNFLKMKRARTTRHTKIEEALLKWFKYQRTNNVPINGPILQQKANDFAQRFGEDFVCSSSWIQRFRARHGIVGGKMSGEAASVDKDAVDEWITQKWPTLCEGYSPEDIYNADETGLFYNMTPDRTLKFKGENCSGGKMSKTRLTIMVAANMTGSCKRKLLVIGKSKKPRCFKNIRSLPVTYENNVKSWMTSEIFERWLRNWDAELKANNKKVLLLVDNCPAHPAVTNLKCIKLVFLPPNVTSVLQPMDQGVIRCLKSHYRRLQVLKLIQSIDSNNQNSFTVLDAILMISEAWEKVSQKTIANCFGHAGFKDLTSLITRTSDDVIDDDEEDNISLAQLAQNLRPALSTTETDEFIDVDQSIAICAPATEDDIVREVQEENENEQEDSEEQFTVPTLIDGLNAVSVLRKIVLFNEEFHMQGNCDDTLIKIQRELQNVYARQKCFKQTKITDFMHTKMMEK

SEQ ID NO: 51 - protein, 505 aa, synthetic construct
MSSCGVKRKVLPIKEKVRLIRAVESGRKIADVCREFGLVNSTVGSILKNKDKILTAFKEDGENVKKIRKCQHSDVDEALLKWFKQCRSANIPISGPILKEKATEFGQMFGSNFTCSNGWLDRFKARHSISFAKICGEAKSVDQNVTSEWVQKTWPTLSEGYKDEDIFNGDETGIFFKLTPDKTLQFKNEKCVGGKLSKQRITAFVCANMTGTEKRKLLVIGKSLRPRCFKNVKKLPVNYTANKKAWMTSAIFEEELRKWDQQLSLKNRKILLLVDNCAAHPKLNLTNINLVFFPANCTSILQPMDQGVIRSLKCHYRKFLLKKIILSMDTAQPVNISILDAIRFLDKSWNNVTPITISKCFRKAQLGSTASLENYDEDDELPLSTWIAQFNVDQSNFGNDIDSYASVDDQVETFQMLSDAEIVADVQKKDAEDSPDEDDDQSAEDFVCPSVQEAMKAAETLSRFFSFRETTASTLSALQDVCCTTERIYFSERTVQKKITDFFRQ

SEQ ID NO: 52 - protein, 518 aa, synthetic construct
MSLKRKRLSIKEKLNIVAESEKYNSSARKLAEKFGVGRTQVNDILKNKVELKKMFEEEGCNADKKRKFPKTEGFAIDQLVYEWFCKARNKNIPISGTLIREKALEVSTSLKLSEFKASTGWLNKFCKRHNINFKTICGESADVDEVTVEDWKTKLTAIIKEYDTKDIFNADETGLYYMAMPNKTFALKNDKCAGKKSAKQRVTLLFCCSMHGEKENPLVIGKSKNPRCFKGAHVDKLPIEWVSNKKAWMTTDIMTSWLKTFDTKMKRQGRKVLLFLDNATSHPKIELENVRIIFFPPNTTSHCQPLDQGIIQNFKLKYRQFFIKRLLTFTNTEETMEKAKESINLASTMVWISTAWKNVTSNTIKKCFQKAGFATVKGDDAFDEEDDIPLSQLSSILKNMPGSPDLKEYLTVDSDVPTENPSLEMQGIIQEFTNNKDRQDSTQAEEEEDEGQDILCSSINNMSEVCEKLRDIQNFLVIQNKDELAVDIAHTLMKFESEITKIKIKNLKQTTINQFFTI

SEQ ID NO: 53 - protein, 499 aa, synthetic construct
MASKRKAISVDEKICVIRAIEKGEKKSDVGRRFELSQSTVATIWKNQKTIQQAALEGNLSKKLRKPKFEDLDQAMLSWFNNQRQNNVPISGPIVKAKAEKFANQLGIIDFKASEGWLGKFKHRHQITYGKMNGEARDVDMNVTNNWISTVWPNLKMKYSPEDIFNADETGLFFKLTPDKTLKFKGEKCVGGKLSKERITVLVAANMSGTEKRKLLVIGKSKNPRCFKNIKQLPVTYKANKSAWMTSQIFEEEVRKWDAELKSRKILLLVDNCPAHPIISNLRNIELAFFPANTTSILQPMDQSVIKSLKGHYRRKMLMDMIETDGKVSINMLQAVNFISKAWQEVTAATIQHSFRHAGLCSSQETNQFNSEDNLPLSEWISQFNVPNNFNEDLQSYENIDEDVATTGTLTDEQIVDLVSKSQETPDNQDEEDDQVDKAESPPTIQQALDAAKLVENFLLFNQDDSTTYLDMNRIHKIIQNKYWHSKKRQTKLSDYMCKQ

SEQ ID NO: 54 - protein, 511 aa, synthetic construct
MSARKRLSLEQKVKLIELYDREKPSVRSLAEKMGIGKTQVADTLKKRENILKLWTENKNKKSKKLLKPGKTLDVDACVLEWFVRVRTNKIPVNGPLIQEKAREVANRLGLPTFKASNGWLEKFRARNNITYRSICGESGSVPEEGVIDWTANLEGLISKHNERDIFNCDETGLFYRALPDKTMTLKGETCAGGKISKERLTVLLCVNMIGEFLPPLIIGKAKKPRCFRGVCTNEIGTDWRYNSKAWMTREIFTQWLTELDEKMGKQKRKILLFLDNASSHYKTENLSNVQLIFFPPNTTSRCQPLDLGIIKNFKVLYRAKILRHLLSKIDKETTVYDLNKSISVLDAIFWTVAAVKEIKPTTVINCFLKAGFKKNINIPSPSQENETNVKEEFTEFVEGITEDYFDIDGSLFTDDQSVDIQQIVKSMIKHDDGDEEEEEDDGDANTQELQPIHVTLPQALESAEKIKHFFLNCEDPVGVKMAADIQMLISTKFTKINQCQTKIEDFFKVVS

SEQ ID NO: 55 - protein, 510 aa, synthetic construct
MAKRKTLSYQDKVKLIETVDAGNKKKSDIAKEFGIPANTLSTILKNREKYERQGTSCKRIKGPEFKDVDESVYQWLKQCRDKNLPVSGPILKEKAAQFAAQLNKPDFRASNGWLQNFKKRNEVIFKKVTGESASVDDNVCTEWSQKLPDLVKGYKPDDIFNADETGLFYQCLPDKTLTFKGDTCHGGKNSKQRVTLLLGTNQIGTVKLKPLMIGKSKNPRCFKGVQSFPMDYTSNKKSWMNSGVFEKWLTDLDRQMKKKKKKILLFIDNATAHGDIPKMKNVKIEFLPPNTTSKLQPLDQGIIKNFKVHYRKEVVRLFLRDLEDKNPTKISILDAMWMASKAWNNVTENTIKNCFKKSGFKQQVDEEEESVVEDCSIVETDNGELASPVLWRQVTQALTLEGLSFEDFVAVDDELATCGQLSDADIIASVSKDTSVEDSVVEDGEDDDDNEVETPDPTLRDARCAINTLRSFLQKKTSMDDKIIQSLSILDDALDKVTFTGRQTTISDFF

SEQ ID NO: 56 - protein, 623 aa, synthetic construct
MPNKYTRKPSGSNRATWSEADLQKAITLVKNRTLNFNQASKEFNIPYRTLRRRFSSGNTKKGRLGPDSLFGDNEKKIVKHIEKLQSRGFAPTINDLRVMAFNLAESMNLKHNFNKDSGKAGYDWVKSFMRRNPEISLRKSEGVSLQRAKGMTKQEVDGFFDLLETLFQDNDFINNPNKVFNMDETGLHLNNKPSHVVAMRGSKTVPQLTSAEKGENISLICCCSAEGNFIPPVAIFKGKNRKPEFADGMPPGSEVYMNEKSSYVNTDLFYKWLKEHFFPRKPPGAVILLLDGHTSHCTSIEMLEFCETNEIILLCFPSHTTHYLQPLDRAFFKSLKSYFNNETNVWIRNNHSRKLGRLQFGDLLGKAWGKSANQNNGASGFRASGIFPPNRNAIPEYAFISDISPEGQADDPSNNNTSVRNIVPSTSGVNKENLAPVANPSTPLKSHNLSEIQSGKEETPSKILNKVHPVPSYGLQVVSKRKQHAEVLTSPENLEKIRNKTNIKQFKTRSKKEPTTTESKMKEPMKRSLSNRNYRKRIKIEQTTSNRKQESNDSCDEESHISLHDSSSDVDIEHEILGDICNVCGEYYHATKKKCDWIQCIQCQQWFHEDCSDDPAKCLMCDD

SEQ ID NO: 57 - protein, 710 aa, synthetic construct
MPRDYKRKTEKKYKLEDLERAVQEVKNKSLSLGKAATVYAVPKSTIHDYLNKDIVKMPQTGRKAIFTEGQEKELEEHILKCSRLFYGLTIEMVRKIAFKFAEVNKLKHNFDKNTAMAGKDWFYGFKKRHPNISLRKPEATSINRISAFNQSEVKIFFDNLEATQKKYQFNANRIYNVDETGISNVQRNSRILAPKGQKQVGMATSGERGTTTTVVCAFSASGKYVPPFFIFKRKRMNAQLLRGGNADMVAAVSDSGWINENLFIDWLHHFISYAKPTVEEPILLVLDNHESHVSLACYLLCRQNGLVLISLPPHTSHRMQPLDLTFFGPLKTAYNKECDLYMASNIGRRITQFEVVELFTKAFNRISNIEKAANGFRSAGIWPLDTTKFDEMFSDLRSPDIQIASASNVQRAVTPNIQMAPSIQRATTPDVQTTLSSFMQRAVTPDAQIAPMALLQKVVAVDDQIDSTSSVQRAAAPDAHIELSQSILKIVTPEAQIAPMVPVIEMVAPDGQIPSSPSLQRAVPPEAQIVRRKSLTVPASPVSNTLTPLRNIISIPQLPQPKNIQNYRKKHSIIITSTPMKDTLEEKEEKKKKKADQKENVKTENIKAERKKKKKGEKEENKTDSTNKRKKGVKNFKGFEDHQVLCKKNKSTKRATENEEVYYCLICNEKYEPPIIEDWIICYKCKLWAHENCTSGESTSRGFLCDFCAD

SEQ ID NO: 58 - protein, 678 aa, synthetic construct
MPRYYKKRTDRTFCAATMKMAVNEVLERGASIRKTADKYQLKKSTLLDYVKRTKMHPENVNYAPDFKKSQILTPCMEEELSDYLVRSSKMFHGLTPACAKRLAFEFAVKNNLSVPENWNTSRSASRDWFTGFMHRHPSLSIRTPEPTSLARMSSFNANNLKLFQDKLEEVLKRYAFEPHEIYNLDETGVTTVPRVPKVISQKGQRQVGQKVSRERGELITHVGIVSASGNALPLVLIFPRHRFDQKRMMQGVPDNGPLGLAHPSGWMTSENFIKVLEHLVKYSKCSIDKKILLLMDNHETHLSIEALDYCKSNGIVVLTFPPHTTNKLQPLDTCIYKSFKAFVSEGCDSWMTNHPGQALTIYDLPFICYSAWDKSATPSNIKKGFQSTGIVPFNRGIFTEEDFLGAYVTDRPLPSETNQSEKRLKNDCHTNEILDFETPSVSRSLHSSPNSTSIVSPSTSIISPENIQPFPKAKERKVTNRGRKRGRCMIATDTPEKNEIAAAKEKSKKNSRTEARPIKKLFETSSKKEATAHPVASSNLCQKDAKKKRNTSSESESDSVLSNIGDSSGGEWQEDTSSDQDNTSVPVGAYAVVNVHGKTTSSGRRYVARIEEENTGGYIVRFLKRKNLSYRFTLSDEDTFFVPYDDVMFKLPSAQTDTRERFKNMIYFNMDLSDFSLY

SEQ ID NO: 59 - protein, 343 aa, synthetic construct
MGRSQELSEFQRGTVIGCHLCNKSSREISSLLNIPQSTVSCIIRTWKCLGTTATQPRSGRPRKLTERGQRMLRRIVRRGRQLSAESIATDLQTSCGLQISSRTVRRELHGMGFHGRAAASKPYITKCNAKRRMQWCKARRHWTLEQWRRVLWSDESRFSIWQSDGRVWVWRLPGERYLSDCIVPSVKFGGGGIMVWGCFSGAGLGPLVPVKGTLNASAYQDILDNSMLPTLWEQFGAGPFLFQHDCAPVHKARSIKTWMTESGVDELDWPAQSPDLNPIEHLWDELERRLRARPSRPTSVCDLTNALLEEWSKIPINTLLNLVDSLPRRVEAVIAAKGGPTSY

SEQ ID NO: 60 - protein, 347 aa, synthetic construct
MARKRELSSETRQSILVLRNEGYSMREIAKKLKISYTGVYYSLQRTAQTGSNQSRKRSGRPRCTTEQEDKYIRVSSLRNRRLTGPQLASSLNSTRKTPVSTSTVKRRLRDSGLQGRVAKKKPYLRLTNKRKRLRWAKEHRHWTEEDWKKVLWTDESKFEVFGSQRRTFVRRRTNEKMLEECLTPSVKHGGGNVMVWGCFGAGKVGDLYRVKGILNKEGYHSILQRHAIPSGQRLIGANFILQQDNDPKHTSKLCKNYLEQKQAAGILSVMEWPAQSPDLNPIELLWEQLDRMVRKKCPSNQSNLWELLLEAWGAISPDYLNKLTARMPKVCNAVIAANGGFFDESKV

SEQ ID NO: 61 - protein, 342 aa, synthetic construct
MAKTRNLTQETRLKIKILSQEGYSCRQIARKCRCSPSAVGYTLQKYRRTNSLEDKPRSGRPRVSSARNDRILIRMCRQNRRMTSQELQQQWSNQTGVQCSTRTVRGRLLDHGLRSYKAIKKPLINERQRLARRRWAQAHKNWTARNWKKILWSDESSFQLYLPPTNVRVRRRPGEALSPACTVPTVKHGGGSIMVWGCMSAAGVGHLTVCDGTLNSTKYCTILETHMLPSARALFRRGKNWMFQQDNAPCHTSKASRTWLQEHSIQVLEWPAQSPDMSPIENLWWIIKRSVSKHKPKNLEELKAVIQEEWDKITPQQCERLVGNMPARIRALLRANGRTTKY

SEQ ID NO: 62 - protein, 340 aa, synthetic construct
MVRAKELSEDFRKKIVAAYESGKGFKKISKDFEISHSTVRKIVYKWRAFKTTANMPRTGRPSKFTPRADRKMLKEVSKNPKVSSRELQQALATVDVEVHASTIRKRLYKFNLHGRCARRKPLLSKRNIEARLTFARDKVDKDQDFWNNVLWTDESKIELFGHNSRGHVWRKPNTAFQEKNLIPTVKHGGGSVMVWGCFAAAGPGQLTIIESTMNSTVYQKVLEEHVRPSVRKLKLKRNWTMQHDNDPKHTSKSTKDWLKKKKWRVLEWPSQSPDLNPIEMLWGDLKRAVRARNPSNISQLKKFCIEEWGKISSDRCRRLVDGYKNRLTAVISAKGGNTRY

SEQ ID NO: 63 - protein, 331 aa, synthetic construct
MGRSRHCSEEQRTLIKKLIREGKTYKEVQKMIGCSAKMISNALKWRAKPERRGRKRKTTIKMDRRITRMAKAQPMITSRMIKDSLELPVSTVTVRRRLCEANLFSRIPRKVPLLKKRHVQKRLQFAKEHINWPKEKWRNILWTDESKIVLFGSKGHRQFVRRPPNSEFKPQYTVKTVKHGGASIMIWACFSYYGVGPIYRIPGIMDQFAYVKILEEVMLPYAEEDMPLKWLFQQDNDPKHTSKRAKSWFQTNKINVMEWPAQSPDLNPIENLWGDIKNAVSEAKPRNVNELWNVVKESWSGITAERCHKLVDSMPHRCQAVLKNCGHTTKY

SEQ ID NO: 64 - protein, 340 aa, synthetic construct
MAPTREMSIETKERIIKLLKEGKSSRNVAKDVGCSQSAVSKLWTKYKQHGKVVKGKHTGRPRKTSKRQDRKLKAICLKNRKCTTKQMRNEWEETGVNVCDRTVRNRLKEMGFTYRKAKRKPSLTPKQKKTRLQWAKEKQSWTVDDWMKVIFSDESRICIGQGDDAGTFVWCRSNEIYKDDCLKRTCKFPQSLMIWGCMSGKGTGEMAVITSSINAQVYVDILDTFLIPSIERMFGDDEIIFQDDNASCHRAKTVKTFLAKRHIGSMSWPANSPDLNPIENLWWKLKKMVHDKAPTCKADLATAIRESWSQIDEEYCLSLIKSMPQRLQAVIKARGGATKY

SEQ ID NO: 65 - protein, 350 aa, synthetic construct
MARTNQLTKEKRVAIITLRNEGQSVRKIAKTLNVSPSGVAKTIKRYNETGTHEDRPRKGRPRVTSASEDKFIRVTSLRNRKLTAAQIRDQMNATQSSSSRPISRTTVKRRLRESGLHGQIAARKPLLRRGNKQKRFVWAKKHKEWTLDQWKSVLWSDESKFEIFGSNRRVFVRRRKGERMDSTCLVPTVKHGGGGVMVWGCFAGDTVGDLFKIEGTLNQHGYHSILQRHAIPSGLRLVGRSFIFQQDNDPKHTSRLCKGYLTKKESDGVLRQMTWPPQSPDLNPIEMVWGELDRRVKAKGPTSAKHLWELLQDCWKTISGDYLLKLMERMPRVCKAVIRAKGGYFEETRI

SEQ ID NO: 66 - protein, 340 aa, synthetic construct
MGKSKELSQDLRNLIVAKHNDGIGYRRISKLLNVPVSTVGAIIRKWKDNHFTINLPRPGAPRKISDRGVKRIIRRVVQEPRTTCGELQKDLQLAGTVVSKKTVSNALHRHGLYARSPRKTPLLKKKHVEARLKFAAQHLEKPVKYWENIVWSDESKIELFGCHNTHHVWRRNGTAHHPKNTIPTVKFGGGNIMVWGCFSANGTGKLHIIEGRMNGQIYRDILDKNLLPSTRMMKMKRGWTFQQDNDPKHTAKETLNWFQRKKIKLLEWPSQSPDLNPIENLWKELKIKIHRRGPRNLQDLKTVCVEEWAKITPEQCMRLVSPYRRRLEAVITNKGFCTKY

SEQ ID NO: 67 - protein, 340 aa, synthetic construct
MGKTKEHSKAIRDKIVEGHKAGKGYKTLSKELGLPVSTVGSIIRKWKAYGTTVSLPRPGQPLKVSSRAEARLVRRVKANPRTTRKELREDLMAVGTLVSVNTISNVLHRNGLRSRRARKVPLLSKRHVKARLQFAHDHLEDSETDWFKVLWSDETKIEIFGANHTRDVWRLDGTAYDPKNTIPTVKHGGGSIMLWGCFSAKGPGHLVRIHGKMDSTAYLEILAKNLRSSIKDLKMGRHFIFQQDNDPKHTAKKTKAWFKREKIKVLQWPSQSPDLNPIENLWKELKIKVHMRHPKNLDNLEKICMEEWAKITPETCAGLIRSYKRRLLAVIANKGYSTKY

SEQ ID NO: 68 - protein, 340 aa, synthetic construct
MGKSKEISQDIRKRIVDLHKSGSSLGAISRCLKVPRSSVQTIIRKYKHHGNVQPSYRSGRRRVLCPRDERALVRKVHLNPRTKAKDLVKMLAEAGKSVSLSTVKRVLYRHGLKGHSPRKKPLLQKKHKKARLQFANAHRDKDLNFWRHVLWSDETKIELFGHNDHRYIWRKKGEACKPENTIPTVKHGGGSIMLWGCFAAGGTGALHKIDGIMRKEHYVEILKQHLKTSARKLKLGRKWVFQMDNDPKHTAKLVTKWLKDNKVNVLEWPSQSPDLNPIENLWAELKRHVRARRPTNLAQLHQFCQEEWAKIPANYCEKLVEAYPKRLTQVIQFKGNATKY

SEQ ID NO: 69 - protein, 338 aa, synthetic construct
MVRKISVEVRKIVMELSLKGKSSREIASVLSIGKSTVKDTINRYRAGHGVKDRPRSGRPRKTTKRIDRIIKRKSIADPKKTASNIATELQQENVANISRCTVTRRLHDVGLFGRVSVKKPLISKKNKVARLKFAQKYCDWTVENWKNVLFSDESKFNLFGSDGRQYVRRSVGTRNDPRYQTPTVKHGGGSIMVWGAFSAQGIGPLIKIEGIMTGVTYKNILETHMLPYGEKEMPENWFFQHDNDPKHASKIVKGWLSTNKVQVLDWPSQSADLNPIEHLWGEMKRRIKNHRSSNKTQFFQNLIEEWNKIPHDYITRLIESMPRRCAAVIAAKGMATKY

SEQ ID NO: 70 - protein, 338 aa, synthetic construct
MARHTQIQIRERVIKFSEEGKSSREIASLLSIGKSTVNDIIVKYRAGYGLKDRPRSGRPRKTSKKVDRIIKRKSTADVKKTAAEIAREIQDENLANVSRSTVTRRLHDVGLFGRIGIKKPLISKKNKKARLQFAKDHQNWTVEDWKKVAFSDESKFNLFGSDGRQHVRRPIGTRNDVRYQIPTVKHGGGSVMVWGVFSAQGVGPLVEINGNMNAAIYKDILERNLLAYAEETMPQDWIFQQDNDPKHTSKLLKQWFSASNVRVLSWPSQSPDLNPIEHLWEKLDRQVRKHTYSNKVTLFKSLKEEWGKIPPACINRLIESMPHRCAAVIAAKGMATKY

SEQ ID NO: 71 - protein, 340 aa, synthetic construct
MGRTELSVSERKIIINLHREGKSYTKIAEVIKRSRSTIQKVVNRFQNENRIENKTRTGRPRKLSERESRYILNEIKKDPKKSAIQISSELREMFGVSVHEETVRRKLNQGGYKSCSARKKPHVSDINRKKRLEFAEKYKSIELDFWDKVIWSDESKFNIFGSDGRVKVWRKPGEALNPKNTIKTVKHGGGGVMVWGCMATSGVGNIVFIDETMDQKVYLNILKENLRASAEKLGLHENYYFQQDNDPKHKAYTVRQWLVFNTPHLLETPPQSPDLNPIEHLWDVLGRKIRQHVIHNKSDLKQVILEEWPKISNDVTKKLVHSMPNRLKEVIKLKGFPTRY

SEQ ID NO: 72 - protein, 339 aa, synthetic construct
MGRRTTIEQRELVIQHYKNGHSRRKIAEMVCLSDSTVQSIIYRFRHENRVLDKGRKAPNKIFNEADERYIVRKIKKNPKLSAPKLVVEVKNELNKNCSAETIRRVLRNHDFNGRVARKKPFINKKNQAARLKFAKEHITKDSSFWEHVIFADESKFNIFGSDGQTYVWRKPNTELQNKNLKPTVKHGGGSVMVWACMSSNGVGNLQFIENTMDKNVYLDILKANLLQSAEKLGVRDEFRFYQDNDPKHKSGIVQTWLIWNCPHLMQPPAQSPDLNVIENLWAILDQNVRQHQISNRNDLIAALKDEWAKITPETTRKLVDSMQSRLKAVIDQKGGHTRF

SEQ ID NO: 73 - protein, 336 aa, synthetic construct
MVRPLLSSKVNEIHHFLRQGKSCSEISLLLNISKSSVGRVRKSLPNLPLPSRGGRKKILSETTERRILRDLRQQKVENAVQIQKQLAENENIHVSVETVRNTLRAAGLKGRAKVKKPMLTKRHQKIRLDFAKKYASWTAEDWENVIWSDETKINRLGSDGKEWCWRKSGQTLQAKDVRPTLKFGGGSVMVWGCMTAKGVGTVHRIVGTMTAQTYVNILSTNLPTAAEKFGLQKEDIVFQQDNDPKHTAKLTKKWLSDNRYNVLDWPPQSPDLNPIEHLWGVLKRGVVGTPGNLEELWAHVREAWIGIDANICKNLVDSMPRRIDAVIKAKGGYTKY

SEQ ID NO: 74 - protein, 342 aa, synthetic construct
MGRKKQETTLAERKICLNLICQGKSLREVAAIIGRTHSTVQSIINKYKNEGQLKNIGGRGRKKLLVPQNEKYIVRQIKINPKKCIKDLTSEISEIINRPISTETIRRTLRKAGYNGRIARKKPYISKVNRMRRLLFAKQYINEGPEFWNRVIFSDESKFNLFGSDGKPIVWRKPNCELNKINLCATVKHGGGNVMVWGCMSAGGVGNLQFVKHIMNKNQYLSILKDNLQPSAIKLGLGANYLFQQDNDPKHTAHIVREWLLYNVPKQLKTPPQSPDLNPIENLWSELDRRLRTRSITSISRLRKCLTEEWLNIPATVTSKLVESMPRRLEAVIKANGYPTKY

SEQ ID NO: 75 - protein, 342 aa, synthetic construct
MGRKGRNTSFEMRQLVIFHREKGKSYREISSMLGVSKSTVSDIVRRYNTEDRIESIPQIGRPLVLNARDRRRIVRKVKCNPCLSALTLASEVFEETKKKVSAETIRRTLREHGYNGRVARKKPYINEANRRKRINYAKEYISKENSWWSNVIFADESKFNIYDSDGRRMVWRKPNTEFRPANMRGTVKHGGGSVMVWGCMSTSGVGNLVFIDGIMDKNAYLSLLKNNLKQSAEKMGISEDFKFYQDNDPKHKARIVQEFLLYNCRKVLHPPPQSPDLNPIENLWDELNRRIRRTPIRSKEELKIGLQDEWSSISIEYVKKIISNMPSRLRHVIKQNGYASKY

SEQ ID NO: 76 - protein, 341 aa, synthetic construct
MPKVPYFSHQIKEKIVELYRSGMKQIAIAKSLNLYRGTVSKIVKKFVSNGNVDTAAKTGRPRKTTQRMDKRIVRMSKKYPRKSASDINKDLQIDDPWLSLSDMTIRRRLIAANLHGRRAVKKPLLRKRHRQARLKFAQDHKDWTTKQWSLVVFSDESKFNLFGSDGVGYVRRPMGQRYDPKYFIPTVKHGGGNIMVWGCFRRAGVGPIAKIDGIMDAQRYVSVLENSLLPFEEEQLPLRWCFQQDNDPKHTSKLAKAWFEDNKIPLIDWPSQSPDLNPIENLWYYLEKEIRKHQIKNKAHLWEVIQEEWQNISPEFCAKLIDSMRNRCLAVIASKGYPTKY

SEQ ID NO: 77 - protein, 334 aa, synthetic construct
MRKLSQDVENSIIGLTEKGCSSRIISSKLGISQSVVIRVQKRRKVSVESPLRGRPRLLNDADARHIMANMRKDKFLTPKGASLEINKDVSRWTARRALQRIGYVAAVKKNKPALSAKNVKARLCFARQHRNWTIDDWKRVIWSDETKFNRFQSDGKQYCWRRPGETIQRHHVKQTVKHGGGNIMVWSCFNWWNIGPMHKIEGIMNKEQYLSILRNHLPEFIDRSAYNEAEIVFQQDGDPKHTAKVVQKWLMDQNFQLMQWPAQSPDLNPIENLWSIVKRRLGQYNSAPSNLANLWERVHEEWRRIPKEILENLVESMPDRIESVIKNKGLWTKY

SEQ ID NO: 78 - protein, 341 aa, synthetic construct
MPRKSNTCFNLRQLVIFHNAKGKSYRQIAAMLNMSKSTVADIVKRFKCEDRIDSIPQKGQPKKLDARDKRKLIRKIKKDPTLSAPKLAAELLNETGKKVHPQTIRRALKDSGYNGRVARKKPYVNEQNRKKRLNFAKEFVLKEEAWWNDVIFADESKFNIFGSDGRRMVWRKKNEEFKFKNLRPTVKHGGGSVLVWGCISTFGPGELVFIDNIMDKNVYLNILKNNLHKSATHMGILNTFKFYQDNDPKHKARIVQEYLLYNCPKVLQPPPQSPDLNPIENMWDELDRRIRKTTITSKEMLKHRLKEEWNNINTEYLKKIIRNMPQRLKEVIQQNGYPTKY

SEQ ID NO: 79 - protein, 342 aa, synthetic construct
MEQKGKEISVEVRKLILKYRLEGKSYREIGLLVGRTHSSCQRVYDNYVSSGSILSKPRSGRPPLLTEREKKRIVQLVPQNPMISAHQGRLEVAERFSKTVSEETVRRTWKKAGYHGRVARRKPHVSEVNRKKRMDFANEHISKPSSFWKRVIFSDESKFCLFGIKGKKIVWRKSGTAMDKENLLPTVKHGGGGVMVWGCMAASGVGNLSFIESTMDHKDYIKVLSENLHQSAQKLHLEDEFCFQQDNDPKHTAHNVKLWLLYNVKSQLRTPPQSPDLNPIEHLWDLLERRIRQHTIRSKNMLKEVMMEEWGKITAEDTKKLVESMPRRLAEVLKRKGYPTRY

SEQ ID NO: 80 - DNA, 349 bp, synthetic construct
  source 1..349: mol_type = other DNA
tacaccggaccctcgctagaacgcgcgtctatataacgcgaattcggatataacgcggtcgcggccatggatcccaaatttaagtactgtacagtacaatttttttgagtatagacgcgataaatcgaccgctgagcgctctcctgtcgactctagtactccaccagaacgaggagcgcaagcgcagtcgacgggagagcgtcagctattcacgtagctgaagttgcgtatcttagatcgaccccgcgcggtagtgtagacaagccctaagacgattgctgtaggcctagaacgccatacaaatgtaatgtaataaaaatttacaggtacagtatttatctttagaaag

SEQ ID NO: 81 - DNA, 384 bp, synthetic construct
  source 1..384: mol_type = other DNA
tacagtggaacctcgcataacgagcacctcgcttaacgagtaattcgcataacgagtaaaacaaattgattcttatagcgagcgatgtttcgcataacgatagttttctaatgacatttcgctgacgtgttttcctcagccagtaagcggccggtgggcgtgaccagacggagtggggcagtcgcagttaaaaggggcagacacagctggagaagtcagtttttgaagtcagtttttagtgagtttttagtagagtttttagtgagtttttagtctttgagagacggtttggaagtgatagttcgcgagagagtcagtgttactgagtgcttcagaaaaaactttacaaaattattgtgtttattttgttatccctctaacaaa

SEQ ID NO: 82 - DNA, 357 bp, synthetic construct
  source 1..357: mol_type = other DNA
tacagttaaacctcgataaagtaataccctcgggaccggaaaaaaatattcatttagcgaggttatttatttatcgataaatgaataaattattcatttatcgataaataaatatttattattttagagagagcttttcaatataaaatatgtacattgtatgtggtaagaaaaatcaaaagacactaatcaaagtattatttgaatctagggaagctcaaaagttttagtgaaacttaatgtctctaaaaactaagttcacgtacatagcaaatgaaaaggtttagtctttacctataaaagctagatgtttggaagacatattccaagcacatacaatattttgtttcccctcca

SEQ ID NO: 83 - DNA, 267 bp, synthetic construct
  source 1..267: mol_type = other DNA
tacagtgcactccgtttataagaattactgatataagaatcaaccgcatatagtgatcaaaaccactgggacaaaatcattcctatactaaccatgttaaaatactccgcttgtaagaatcaagcaatccgcttataagaatcatttcggggcaaactgactacatgtaatacgatactcaatcaagtgcaatttaggcacgtagcgcgtataaagtataatccctgtgtagaaaaaacaagtcgtgccgtttatgtgtgagtcgac

SEQ ID NO: 84 - DNA, 224 bp, synthetic construct
  source 1..224: mol_type = other DNA
taacggatattaccgaataatatgactacttcaaatagtatgaccaatttttttatggtcatactatttaataatatgactataattacatatacatttgaagttaataaattgatatttaatctacaaccagcaatttttatgtattattatgttaattcgaaatatctgcataattaacatttcaaaaaattttaatatttaaaagaaaacaaacactttcc

SEQ ID NO: 85 - DNA, 256 bp, synthetic construct
  source 1..256: mol_type = other DNA
taccgtatatttcggcgtataaggcgaccgtcagataagacgaggtacaaaattagagcaaatttttatgatttatcatatatcggttgtataagacgactgctaaataacgaaaccatgtttgtttagcctaggctagagattgcaacacccggtatcttatgaaatattggttatgtaaagatgatttattagtatagattcagtatagattggtatagattgtggtcattttattattaaacccatatcagga

SEQ ID NO: 86 - DNA, 292 bp, synthetic construct
  source 1..292: mol_type = other DNA
taccgtaatttccggactataagccgcaacttttttcccacgctttgaacctcgcggcttaaacaatgacgcggctaatatatggatttttcccgctttcaatttttttttctccaaaaaaacacattctgtgacgtgctcagttttttggcggcatgaagctttcattagaccaatgaaattgccgaacgggttaaggtcaaacaacttttttgtttactgtttagattaaatcgagcgctctcaaacttcccatcattctgattacggtagtcattttgtcaccctcatc

SEQ ID NO: 87 - DNA, 263 bp, synthetic construct
  source 1..263: mol_type = other DNA
taccgtattttccgcactataaggcgcacctaaaagccttaaattttgtcataaaacggccgtgcgcctaataatccggtgcgctttatgtgtgcacggagttcaaaaatctgtaaaaatgttgttgtgtgatttcggaaagcgctccgcttgattggctgtcggagcatttcccgccgacacagagacgtaaaacgtacactccgtatgcagcagcaataaaccaatcaaaaccacatatgtttgtctaatgatccccgaaa

SEQ ID NO: 88 - DNA, 710 bp, synthetic construct
  source 1..710: mol_type = other DNA
tagggtaaaggccggtatcttcggacagccggtaaattcggacaggactcgttaaattcagtcctttaccttcaacaaatacacacttgctttctctttttcactttcggttaatattagaaataaagttgtacgacttaccttttttatccggatgtgttgttgttattatttaaaaccagcaaaaaacaaaacttctttttccggattgatacaacagttgtcgaccgcgataatttcaaggactcgttccaaagttttgattaggtaagtaatatttttttttcactcaatacacaattttcccaaacaatttgattatcatattgtgcttttaagggaaatgaacatagttttaagtgttaaatcatgctgtttttaaagcaacattggtattttatagtgtccgaaattaacggacgaatttttttcaaagtcatttaatttcggacattttttttaaaatgtaagaaaaatagaaagagaattttttatcgttagtttgaaaatttatcaagtctattaacggtaaaacattttcaaaaacaatttgcaactatattttttccagatttttctagataattaattattcagtaacgtttgcttttttataattatacatctatgttgattttttttaaatgtctccgatgctatttattaatttttattgtttgtttttttaatagggaaaattttctttcagc

SEQ ID NO: 89 - DNA, 512 bp, synthetic construct
  source 1..512: mol_type = other DNA
taggggaggtaggggagcattgggcagttgggaggtttgggcaccccctcatttctcacaatcttttctagatattttttgttttttaccatagcattatagttttataattggcaacacttttcatcaaaagtgtcagctgtcgtttcacttggttgtcaagttacatgcgcttaacagtttgttgcgttcaaaagtaaatattttgtttatgatttgaagaaagataacaaagaaatctgcaagaaattgtaagtatctgtgttaacttcttcttttgtgtttaatgaacattatatttacctgacaattaggattcaacatttgataaaaataaacttattcaaatctgtttaaaaagtgaggttatggggagcattgggcacgcaggtaggagggaggtttgggcatgcccaatgctccctctttttttagtgtgtttttttgtcatatgtttgtgtttttttgctatgactattggattgaatggttaatcatttcttttcaggaca

SEQ ID NO: 90 - DNA, 381 bp, synthetic construct
  source 1..381: mol_type = other DNA
tacagtggcttgcaaaagtattcataccccttgaacttttccacattttgtcacgttacaaccacaaacgtaaatgtattttattgggattttatgtgatagaccaacacaaagtggcgcataattgtgaagtggaaggaaaatgatacatggttttcaaatttttttacaaataaaaaactgaaaagtgtggcgtgcaaaagtattcagccccctttactctgatacccctaaataaaatccagtgcaaccaattgccttcagaagtcacctaattagtaaatagagtccacttgtgtgtaatctaatctcagtataaatacagctgttctgtgaaggcctcagaggtttgttagagaacattagtgaacaaacagcatc

SEQ ID NO: 91 - DNA, 370 bp, synthetic construct
  source 1..370: mol_type = other DNA
tacagtggtgtgaaaaagtgttggcccccttcctgatttcttattttttttgcatgtttgtcacactttaatgtttcagatcatcaaacaaatttaaatattagtcaaagataacacaagtaaacacaacatgcagtttttaaatgaaggtttttattattaagggaaaacaaaatccaaaactacatggccctgtgtgaagaagtgtttgcccccctgttaaaactgtggtttatcacacctgagttcaatttctctagccacacccaggcctgattactgccacacctgttcgcaatcaagaaatctcttaaataggacctgcctgacaaagtgaagtagaccaaaagatcctcaaaagctagacatc

SEQ ID NO: 92 - DNA, 391 bp, synthetic construct
  source 1..391: mol_type = other DNA
tacagtgctgtgaaaaagtatttgcccccttcctgatttcttatttttttgcacatttgtcacacttaaatgtttcagatcatcaaacaaattttaatattacacaaagataacccaagtaaacacaaaatgcagtttttaagtgataattttatttattaagggaaaaaagctatccgaaccttcatggccctatgtgacaaagtaattgcccccccttgttaaatcatgaatttactgtggttaatcacattttttggaaagctgagttcaatttcactagccacacccaggcctgattactgccagacctgttgaatcaagaaatcacttaaatagaacctgtctgacaaagtgaagtaggccaaaagatctcaaaaagcaagacatc

SEQ ID NO: 93 - DNA, 381 bp, synthetic construct
  source 1..381: mol_type = other DNA
tacagtggtgtgaaaaagtgtttgcccccttcctcatttcctgttcctttgcatgtttgtcacacttaagtgtttcggaacatcaaaccaatttaaacaatagtcaaggacaacacaagtaaacacaaaatgcaatttgtaaatgaaggtgtttattattaaaggtgaaaaaaaatccaaaccatcatggccctgtgtgaaaaagtgattgccccccttgttaaaacatactataactgtggttgtccacacctgagttcaatttctctagccacacccaggcctgattattgccacacctgttcacaatcaaggcatcacttaaataggagctgcttgacacagtaaggtccaccagaagatccttaaaagctacacatc

SEQ ID NO: 94 - DNA, 382 bp, synthetic construct
  source 1..382: mol_type = other DNA
tacagtggcaagaaaaagtatgtgaaccctttggaattacctggatttctgcataaattggtcatcaaatttgatctgatcttcatctaagtcacaacaatagacaaacatagtgtgcttaaactaataacacacaaattattgtatttttcttgtctatattgaatacatcatttaaacattcacagtgtaggttggaaaaagtatgtgaacccctaggctaatgacttctccaaaagctaattggagtcaggagtcagctaacctggagtccaatcaatgagacgagattggagatgttggttagagctgccttgccctataaaaaacactcacaaaatttgagtttgctattcacaagaagcattgcctgatgtgaacc

SEQ ID NO: 95 - DNA, 358 bp, synthetic construct
  source 1..358: mol_type = other DNA
tacagtgagagtaaaaagtatttgatcccttgctgcttttgttggtttgcccactaataaagacctgatcattctgtacttttaatggtagatgtattctaacatggagagacagaatatcaaaaagaaaatccagaaaataactttaaagaatatatattaattgatttgtatttcattgagggaaataagtatttgatcccctagtatgcattaggagttctggctttcacagaccagttagacactcccaatcaacttgttacctgaattgaagacacctgttctaactaatcacctgtgtgaaagacacctgttcacagaatcagacaatcagacagattccaaactctccaac

SEQ ID NO: 96 - DNA, 370 bp, synthetic construct
  source 1..370: mol_type = other DNA
tacagtggggagaacaagtatttgatacactgccgattttgcaggttttcctacttacaaagcatgtagaggtctgtaatttttatcataggtacacttcaactgtgagagacggaatctaaaacaaaaatccagaaaatcacattgtatgatttttaagtaattaatttgcattttattgcatgacataagtatttgatcacctaccaaccagtaagaattccggctctcacagacctgttagtttttctttaagaagccctcctgttctccactcattacctgtattaactgcacctgtttgaactcgttacctgtataaaagacacctgtccacacactcaatcaaacagactccaacctctccaca

SEQ ID NO: 97 - DNA, 368 bp, synthetic construct
  source 1..368: mol_type = other DNA
tacagtggggcaaaaaagtatttagtcagccaccaattgtgcaagttctcccacttaaaaagatgagagaggcctgtaattttcatcataggtacacgtcaactatgacagacaaaatgagaaaaaaaaatccagaaaatcacattgtaggattttttatgaatttatttgcaaattatggtggaaaataagtatttggtcacctacaaacaagcaagatttctggctctcacagacctgtaacttcttctttaagaggctcctctgtcctccactcgttacctgtattaatggcacctgtttgaacttgttatcagtataaaagacacctgtccacaacctcaaacagtcacactccaaactccact

SEQ ID NO: 98 - DNA, 377 bp, synthetic construct
  source 1..377: mol_type = other DNA
tacagtgcctagacataagtatttgcacaactctattttttatgtattttaaaaaattcagggactgctttaggataattaatacaggaagtttatataacatatatataatagaatatttaaacagaacagtcaaatgtgcacttataacaagaaaatttaacacaagtaaaagtaatgagggacaaaagtatttgaccaaaataaatatgatgttgaatctttgatatttaatagtataatttgaataaattagtttggaaattattctagaacgttcacgaaacgtctcgaaacaaatgcaatgtaaacaaaccacagccggcgcaatcattacctctctagacctcgttgaagtcgcgcacactctcactaca

SEQ ID NO: 99 - DNA, 389 bp, synthetic construct
  source 1..389: mol_type = other DNA
tacagttgtggtcaaaagtttacatacacttgtaaagaacataatgtcgtggctctcttgagtttccagttatttctacaactctgatttttctctgatagagtgattggaacagatacttctttgtcacaaaaaacattcatgaagtttggttcttttatgactttattatgggttaacagaaaaagtgacaaaatctgctgggtcaaaaatatacatacagcaacatgaattagcaattttggtgacttagaaagttgtgtcaatgaaatgagcttcatagctggcctcttaacttcttgtgagtgattatgagtgactacagctggtgacttctctgaggccatttaaatagggctcattggatacaaacgcccacaaacgctaca

SEQ ID NO: 100 - DNA, 400 bp, synthetic construct
  source 1..400: mol_type = other DNA
tacagtcgtatgaaaaagtttgggcacccctctgaggctgcataataatttactctgtcgtcacagaaaatgatcacagtggcatgccattcattttctaataaaagctgagtactggggtattgtccagacaaagatttttagtgtagcaatattaagttgtatgaaattaaatcagatgtgaaaaataggctatgcaaaaatttgggcacccttgtcattctgttgatttgaatacctgtaactacttagcactgattaattggaacacacaattggtttggtgagctcattaagccttgaacttcatagacaagtgcatccaatcatgagaaaaggtatttaaggtggccaattgcaagttgttgttctctttgactctcctctgaagagtggcaac

SEQ ID NO: 101 - DNA, 380 bp, synthetic construct
  source 1..380: mol_type = other DNA
tacagtatctcacaaaagtgagtacacccctcacatttttgtaaatatttgagtatatcttttcatgtgacaacactgaagaaatgacactttgctacaatgtaaagtagtgagtgtacagcttgtataacagtgtaaatttgctgtcccctcaaaataactcaacacacagccattaatgtctaaaccgctggcaacaaaagtgagtacacccctaagtgaaaatgtccaaattgggcccaattagccattttccctccccggtgtcatgtgactcgttagtgttacaaggtctcaggtgtgaatggggagcaggtgtgttaaatttggtgtcatcgctctcacactccctcatactgactggtcactggaagttcaac

SEQ ID NO: 102 - DNA, 368 bp, synthetic construct
  source 1..368: mol_type = other DNA
tacagtactggacgaaagaaatcatacacctttaaaaaaattgattttgttagtgtaactatttttttaacaagcaaatagattattaaatacaggaaaatcaaagcatttcaaagtatttaaataaaggaaatatgaaataatcaaaattgttctcaacttttttgtaaaaaattgattatttaatattgttacattttaccctggacaaaataaatcatacacttaaatagtttaacgtagggaactgccaatgtttacatttcgtcgttctgctattgttcgtcattctactgtttgtttacattgtatctacgcttagaatttacgtgtttagtgaatcagagtaactgtaacgagcaagaaca

SEQ ID NO: 103 - DNA, 338 bp, synthetic construct
  source 1..338: mol_type = other DNA
tacagttagaggaaaaagtttgcgtacgagatgtttcaagaatttcaatccgttgtcatttattcattttttggagtatgatgtccaattttttttcgtagcatgctaaatttatctaagtttctcactacgtacaaaatttccccacaaagttgccacttttagcgccattaatgtatttccgcatgaaggctgaaaaacacgaggaaaaagtttgcgtacgaattgggtaatttttaatattcgcgacgaccatttttttcggtaagcaatttttttataaattgtagattagattattgtctgtagtttacaacaaaaactttttgatttaagtc

SEQ ID NO: 104 - DNA, 317 bp, synthetic construct
  source 1..317: mol_type = other DNA
tacactagcgggcaaaataagagaacgattttcattatttgaaaataaagtttaaccatgtagagtaggttaatgaatcttttgatggggccttaaggaatgtatttaacatatttctacaaaatatcaggacaaacataactatatttaatacattagagcaatctaacaaacaataacaaaaattgaacagtgcaaaataagagaacgatcgtctctaaaacattctcacatttaccccggtcatctggcaacattcaatttcgtgtacatgacttctagaactgccactcttacgattacaccattctagaaac

SEQ ID NO: 105 - DNA, 313 bp, synthetic construct
  source 1..313: mol_type = other DNA
tacagttgcgagcataattaagggacaaaaataaaaaatttcatatctcaaaaactgtaaaaggttcaacgctgaaattttaacacaaaagaggtattgggatacctaataatatgctaagaatttattacgatccgaaggataagaaaaaagttatgtatatttaacttataattatgaatttagtgtgagcagaattaagggacacttttcaaatattgtaatttaaatcttcagtaaccccattttcttgcgtttccgaagaagaagaagactgcctacgtcatatgtaaacattgcaagtactgcaagc

SEQ ID NO: 106 - DNA, 345 bp, synthetic construct
  source 1..345: mol_type = other DNA
tacagctgttttcaaaataataggagtgcttttgcatatgcacttgtcccttctgtagcaatgttaggcgttcatttttttcaattttatttctttttccaaatttgtttatgggtacacattctaaggtaccgttattttttcgttttactatcttcgttttctgagaattacgcaaattcgtaaacaaacttcattttgcgttcatcaaaataataggagtgtggcattttaatctgctcagagcgaaaaaataggtaattattcttaaaaaaacggtttaaaagtatagaaagattaatttccaactacaaatttaacgtttaagatcgaaaacttaaagca

SEQ ID NO: 107 - DNA, 305 bp, synthetic construct
  source 1..305: mol_type = other DNA
tacactatcggacaaaagtaaggcaacattttgaaaaatatagaaattaatgtctatctaatgtaaataaattatgatcgggcaaactatggtattattaaaagaatcaaaggaagctactgtcattaatgtcaagtaattatttattaaggttgaaaaaaaccgctttgttgacttgtagtgtaaaaaaggattattttaagacgacaaaagtaaggcaacatttagatttttttcaaaaaatgttgttataatgaaaaatttgttaggccaatattaacagttcgtcttgtgcaacaagagcc

SEQ ID NO: 108 - DNA, 298 bp, synthetic construct
  source 1..298: mol_type = other DNA
tacagtagcggcggaaagtattttgacaacgcgttttttgcagataaagcgcacgccgggcgcgctgttgattagaagttgttgttagtgaacacttttctgcagtctgcagcggtgacaacatcagtcttgattgagtggatgtttcgtttcggaagtgctgctgttatctttaatctgtaagtcgaaaagtattttgacacagaaggttttgtctaaaagagacacgcaggggctcacgtaagttactttatttacggtttttgtttactttatgttttgtttatgttcaggagct

SEQ ID NO: 109 - DNA, 376 bp, synthetic construct
  source 1..376: mol_type = other DNA
tacagtcgtggccaaaagttttgagaatgacacaaatattaattttcacaaagtctgctgcctcagtttgtatgatggcaatttgcatatactccagaatgttatgaagagtgatcagatgaattgcaattaattgcaaagtccctctttgccatgcaaatgaactgaatcccccaaaaacatttccactgcatttcagccctgccacaaaaggaccagctgacatcatgtcagtgattctctcgttaacacaggtgtgagtgttgacgaggacaaggctggagatcactctgtcatgctgattgagttcgaataacagactggaagcttcaaaaggagggtggtgcttggaatcattgttcttcctctgtcaacc

SEQ ID NO: 110 - DNA, 405 bp, synthetic construct
  source 1..405: mol_type = other DNA
tacagtggcgagcaaaattgagtgtatgttcagaactcagactttcgtcacccacagaatcataaccacacaagataatcccaaaattttttttgttttatattcattattttattcttaataaattttaagcaaaaacagtcatgccagagataaattctaggagaataacaaaaaaaacgaaaaaaacccgcataaactcaattttgcacgtacgcactttttgcctattttcgctttctaacttctcttatcagcacaaatcggcaagattcaccattatttttgattttctattattcttaatgttcgtagcacagattaaaacgtgcttcagactaagcagagtgtaactgaattagcttataattagaaataaatttttcaaaatttgcgagcataaaa

SEQ ID NO: 111 - DNA, 397 bp, synthetic construct
  source 1..397: mol_type = other DNA
tacagtggggagcaaaagtgattccatgttcagaatatgcatgttgaaccgctcataaaacgtaaaccaatgaaccaaatgaaacaaactttttttttctcgattacttatttaatttgtaagaaatagacttaaatactacaataatacaagaaaaaatgtaagagatagaaaaaaaaaacaaaaacagaaggcatgtactcagttttgcaccttccaatattcgcacctgattttgctctccctgttttcttatcaccaaaatatttgcagatttactgttttatgtctttttcatgcattagttgttgtcataattcaaagagtcgcacatttcttatagttcaaagtaaattttaataaattgagaaattagtcgccaacttatatttgtaaa

SEQ ID NO: 112 - DNA, 332 bp, synthetic construct
  source 1..332: mol_type = other DNA
tatactctcccaaaaaagaaacgcaacatgtagtatcatttttgcaaaagaaatgtttgtatataaaataactgaaagcaagttttgtaatgatatgcttaggtttccatattaccagggatgaatttaaatggcaaaatctgatgtgcgatcaaccgtaactgttgcgaaattaaaaataggaacattgttcgattggtgtttcaagaaagtggaaagcgctttgcacgcctaaatttttgttaatttttatgtgtgatgtgtgttaacaaaataagactatcgcatgaattaaatgtttgcccttgttagctgtaggacgatttttacgt

SEQ ID NO: 113 - DNA, 279 bp, synthetic construct
  source 1..279: mol_type = other DNA
taaactcagcaaaaaaagaaacgtccctttttcaggaccctgtctttcaaagataattcgtaaaaatccaaataacttcacagatcttcattgtaaagggtttaaacactgtttcccatgcttgttcaatgaaccataaacaattaatgaacatgcacctgtggaacggtcgttaagacactaacagcttacagacggtaggcaattaaggtcacagttatgaaaacttaggacactaaagaggcctttctactgactctgaaaaacaccaaaagaaag

SEQ ID NO: 114 - DNA, 270 bp, synthetic construct
  source 1..270: mol_type = other DNA
tacacaaagaaaaaagttaagcacatccttaaacaaatcatgatagttttgcaccaagttttaataatttaataaatatgggatttttgaaaagtaaaatgtgcctttaataaagtggagtgaacaaatcatgacaaaacatctcgtggagaacatgaaccgcacgtgcagtttgctacccttgtgtttctcaaaatgcagtatcttttgtcgcttctattggtttaattctctcattcaaaatcgatctttactcacacaaagggacat

SEQ ID NO: 115 - DNA, 349 bp, synthetic construct
  source 1..349: mol_type = other DNA
taaagtggtggcaataatgatagcacaattcagaatttttcgataacttaaaaaatactacgccatatcatgtgaaattttgacataaaaaccttatttcaactagttacctatttcttatcatagtttccctataaactatttgattaaacttttatgatgaaaagtataaatttgtgttggaatttgggtggaaataaagatagcacaacttggaaaaaaattacatttccgcgttactacatccagattagagaagaagaatatcaaaacaaaccggcgtcgccatattggacagttgctccccaccttgtaacctctatatttggaaattaacccatttcttagc

SEQ ID NO: 116 - DNA, 185 bp, synthetic construct
  source 1..185: mol_type = other DNA
tatcaggttgtctaaaaagtattgagccttttgcgcacgagatagcgttagtgtcccaaatctcagggtatatgagtcgcagcaggtcaggcctaacgtcatttgacagctgacagttgaagctagtagacgtacaaaaagtgttggaattcttataatcagacgtaaggtattgcgccgtgaag

SEQ ID NO: 117 - DNA, 267 bp, synthetic construct
  source 1..267: mol_type = other DNA
tacacctttgcccgtccttcttgggaatagttgcaagcaaagtgaacaccgcacatgtccttaacgcaaatatcacatcattggctttcaaatcaaggtccaatgtgagatttattgcatccaatgtggaacggccagaaacgactggcttcagcgtgctcccgccaataattcaatgactggtgaggatactcgcgcgccaaaacgaacgcaaaaccacttcaccaggcttcacgctcagtccccaacccacctcataagacgcag

SEQ ID NO: 118 - DNA, 178 bp, synthetic construct
  source 1..178: mol_type = other DNA
tacagtttttttcttttggttttcctcttttgtcaaatgtatttcactatttatcaaaagtatttcccatctctattaaacagtttgctgtgtatgtcaaactatttccccttgtgtcaaatctatttccttatttgttaaatctatttcacattgatattaacattgattattaaga

SEQ ID NO: 119 - DNA, 676 bp, synthetic construct
  source 1..676: mol_type = other DNA
taccgtattttccggcgtataagacgactttctagcacctaaaatcttttttaaaaagtcggggtcgtcttatacgccgggaatgtggcagcaaggaggaggggtggccgcactgaacaaacacttcacacatttgcagaaccctaaccctaacccagtgctgagatctaatataccaggctttgtgaacttccatgttaaaaggaggccaagggagaaccaagcaagacaattaaaccaacggtcggaggcacttcaacacaatcagcagaggacagttgtgaatgcaatgcattaacaggcgcgtgagcacggaatataaatcagacaactggctttaacactgacatacgaagcaaaaggcagtacactacctcccattcgaaatatgggcaccacaccccgaaacactacgatggtgcttcattttcactttataaaattaaagacatcacacggatcacgagagctcctgacttgccaagacaacgaattcaaaacgtggtcacgtggtaataacggccgtggggccacgtgagctctacggaaaccctcccctacttcccccttccactctcacctcccctacacctttcaatatttctctcagatggcgttggtgcaagctacccggaagccccataagagatcgcactggttgtaatataccgtataa

SEQ ID NO: 120 - DNA, 284 bp, synthetic construct
  source 1..284: mol_type = other DNA
taccgtatttaccgcactataaggcgcacgtaaaatccttaaatcttcacaaaaatcgacagtgcgccttataatctggtgcgccttatgtatgaattttggtttgtgcttactgacctcgaaccggttttatgtggcacactatgctcaaaaatctgtcaaaaaaagatttttagtgccactttttggagcattacggttaccgttgccaggagcctcgcggagtaatacgtactgtgcttcaacacaacattaccgtgttgtgtgtgtataacgactccaaa

SEQ ID NO: 121 - DNA, 248 bp, synthetic construct
  source 1..248: mol_type = other DNA
taccgtactttctggaccataagccgctactttttccctaggttttgagccatgaggcttatacaaaggtgcggctattctgtggatttttcttccaccactagggggactctaaccagaattagaataaaaaacaagatagatgaaaaatcaatgcaaagaagaatacactactttttctttagcagatagaacacgcacaacaaattaccaactggtaattattttcaaatcctcgccccttcatc

SEQ ID NO: 122 - DNA, 351 bp, synthetic construct
  source 1..351: mol_type = other DNA
taccgtatttgccggtgtataagacgcacttttgatgcttaaaaattcatcttaaaaatggggtgcgtcttatgcaatgaaggtaattattttccacctcgcatacgcactccctttgaaattccaatatctttattggcggttagtgacgctagtatgcggtacttccccgctttggacggttgagctctgcatctcgtgggtaaccgacaaaataccggtacgtcattggctgagagcggggagtccctatcagttacaggtttgttgttctgtgatcagttaccgatgtgtatcagtattgagttaatgtgttgtgcgttacgtgatatttccgcgtagtttttagtg

SEQ ID NO: 123 - DNA, 225 bp, synthetic construct
  source 1..225: mol_type = other DNA
taccgtattttccggactataagtcgcagtttttttcatagtttggccgggggtgcgactaatactctggagcgacttatgtgtgaaattattaacacattattaccggtatatcatttcacatgttattttcacactaaaccgcaagagggcgctctaggcctgtgttgataatttcaacattggcggtaacttataaaaacaactgagaagggcttaacaaaa

SEQ ID NO: 124 - DNA, 390 bp, synthetic construct
  source 1..390: mol_type = other DNA
tacagtagaacctcgattaaccgtcacccttttaaccgtcagtctctattaaccgccaccccgctagcgcaaaaaaattgtatgtttccgcgtcagcgccgagccggatcgtctgtcgaggctctatgatgagtagtgacgactgtactgcacacctgcactgtgcgtgagtcattggcggcaagggcagtgaccttgggtttgcgcgcccacccccccttacactgtcttctgcattccactgctactgtttcagttcagtacggtgcgtggcttactacagtgaaccgaagtttttgccgatttcgagtgcagtgcctactagtgttcgtgctttaccgtttattgttgacagtgctgtttatacgtagtgtgggttgctcttctgtt

SEQ ID NO: 125 - DNA, 317 bp, synthetic construct
  source 1..317: mol_type = other DNA
tacagtaataccccgctttacgcgaccccgttatgcgcggtttcactataacgcgaatttcaaatttcgcgctcaaatgcctctcattacgcgaaagagtgcgtataaacataatgtagtcatcaacttctctatggtcgcgggaatccccgcttcctctgaacattctgtttgaagaattgggaattccccgtccttcgactttgttctatggggattccctcgttcgtttcgtttgttccgtcatgtttagttcggctggcagtcgtgttttaacatttattggagatatgtcataatttaacgctacgtcaaaa

SEQ ID NO: 126 - DNA, 254 bp, synthetic construct
  source 1..254: mol_type = other DNA
tacagtagccgcccggttatccggaatcgggttatccgaaattcgaattatccgaaacgaagttagaaatcgatttcgccgtattatccgaaataggttttaacttgccgccgcattatccgaaattggacgtgtttctttcgtattctttcctagaaacaataaaaatgtgttttgttttggtctccgcgtcgcacagtcgagattttgccagtatcaactcgattctacggtgttaggttgttaaaaaatta

SEQ ID NO: 127 - DNA, 397 bp, synthetic construct
  source 1..397: mol_type = other DNA
tacagtgtaaactccatgtaacgtcactctatttaacgacaaactctcttaagcgtcgaaatcaattggctttggttggtttagcttgtttttcatacaatgtttcactctatatagcattacacccactctcaataacgacaacaattaagttgctaaaattagtaaaaactgcgcagctgtgtacgatctattgtcgctttattatcctagcccgcaataaactcgactgtcggcatcatgcataccgaactttctaagaaattcattcttttgtttacaaattgggattgcttgcacgtgtagactgttattgaccatgattcataactaggagtcatggattatctataccttatcatattcttagttgctcacaaacttgcaaactgtaaac

SEQ ID NO: 128 - DNA, 464 bp, synthetic construct
  source 1..464: mol_type = other DNA
tacagtcaaacctcgttagtacgaacctcggtaaaacgaattcctcgataagacgaattttttaaaatccccttgaaacttccataaaggtcaatgctaaaaacacctctatacaaagcagtaattaggttaaaaacctcgataagacgaatttgtttgtgacatgggctaggactgattaacctctatacaacgaatattcttagcacaagacctctacaagacgaatgttacctctttaagacgaatttcttatgatcttaacctccatagtgcgtagttttcaaaacaaacagaagcgtgtcttaacatgaaactccccaccctaccctcttccttcatcagtcattgtttggcacgtgcctttgcagtgttgagtaacccttccggctgtgtttttaagtgtcccttccggctgtgttttaagactgaccgttgtgcgtatctttattgtatgaacagta

SEQ ID NO: 129 - DNA, 442 bp, synthetic construct
  source 1..442: mol_type = other DNA
tacagtcaaaaccgtttatggcgacatcgtttagaacaacataccggttaaattgaccaaaatcaaaggtcctggctgaatgttatatgaccgccttatcgaaaacattggtttttacgacattgtttactacgacataccgcattaagcgaccacatttggttaatgttttcggagaattatatctgtagaacgaccggctcagctgtattgtaaacaatttgaataggtaactgtatccacatctggcacagtataatagtcaatggctattatcgtaaaagtaaacaaagtttagggtcttttgtaattcttagaaacaaagcacatgcatgccgtagcctcaaggcccgcttcgtcatatctcttaagtcagtttgttacttatctttacacaagacgcaccaaaacatattgttgagttaactgtgaaaattgtaac

SEQ ID NO: 130 - DNA, 527 bp, synthetic construct
  source 1..527: mol_type = other DNA
tacagtggaacttggttataacagcatcggctgtaacgtcaactcgggtttaacgtcacattttatacagaccggccaaaattgaacattttatgttgtacgaaaacccgtttatatcgtcaacaaatatcgcggcgctcggctatagcgtcaaaaattttgcgattcttaggttgcaaaagtggtagtgtgcttgtattatgtcccaaagttcatagaaacaatgtgtagaaacaacaaaagcaaaaacaggtcacaagccggacgttgagctggtgacgggatgaaactcttttgtgtaattggtgtctcgagggggcgggggctgtcccgcatacctgggtcttatcccttcccacccctacattctacaaggtcaatgactgtggtgacacaggcacactgtactgtattgtttgtttcattagttacggtaaatcatcagtcacatcactacctacctccgtgtgagcatcgcgtgtgttgacagttgttttatacgtcgttagttgtgttttgtttgtgca

SEQ ID NO: 131 - DNA, 408 bp, synthetic construct
  source 1..408: mol_type = other DNA
tacagtaaaacttcccatttcggccacttctaaaaagcggtcacttctgttagcggtcaattttcagttgtttgaattaatcttaggggaattcctatattaaaatatttctcaataacgttaattccgaaagcggtcaccggccgcttcaaaggtacattttagggaaaaacccattaaatttattgccccaataccggccaaaaaccgaacccttcgaacctttttttttgtctaacaagagctgtttcaggacactttaggaaattccaagcaataaacatttataatttgtttgagaaataggtacagagagtgacaaatacaatacaaattcgtcagtagtttgctgaaatttttgggagatatagaatcagtttagtttcttactctgaaagaacagcaacc

SEQ ID NO: 132 - DNA, 501 bp, synthetic construct
  source 1..501: mol_type = other DNA
tacagtgtaaactctttataacgttatcctttataacgataaactccctataacgttggagtgagcccaagttggttggtttgcgttaaaactcacatattgaaacactctttatagcgatacgccattctctattacgaagaaatgtgttcatatttgtagacagtaaatatttattatcggtattattgtttatgttatgttatcaggtaacaaaagactttgaagctccgaaacatccgaggtcggtctagcttttgttatccttaattgccttgtacacgtgcagtatgacgcattgttttctcggaagtgatgttatcaggtaacaaaagactttgaagctccgaaacatccgaggtcggtctagcttttgttatccttaattgccttgtacacgtgcagtatgacgcattgttttctcggaagtgatggtcttcgaccaagcatactgggtacctatcgtattatcagtcgtaaacctactttgaaactgttaac

SEQ ID NO: 133 - DNA, 390 bp, synthetic construct
  source 1..390: mol_type = other DNA
tacagtgaaacctctactaacggacacctctaaatagcggacacctctaaataccgaacatttttcaatgtacgggcactaagatgggatttgtaatgcttttgatcctctaaataacggactctctaaataacggacgcggacacggaaaatatacacaattggttcagaaagtatgaaaatgtacctccaaatagcggacagcaaggcaaaagtgaattaaatttcgaggaatacatgtagtggagcccgcaaacctacccacggctgcttcttggaaaattcgtcagttttgttgacatttttttcagcttcccagttctcttaattccgttctaaccgaaaacattgcttcagttatcgtttggctgttgatggaaatacactgtc

SEQ ID NO: 134 - DNA, 501 bp, synthetic construct
  source 1..501: mol_type = other DNA
tacagtaggaccttgatatatcgaacctcaatatatcgaattcctcgataaatcgaagttttgtcattccccttgaatgtcccatttacttcaatgctaattttacctctacatatcgaagatttcaagtaaataacctcaatatatcgaattttcgccaataattttgcctattttttacctctatatatcgaagttttcttataattacctcaacatatcgaagtttcagtttatcaacgaaacgaactttagtactgtattgtcgtgttgtgcaaacgaacaaccatcaaagtagagtaggtaccgtatttttcatgtcgagacttgaaacccccaccaccgacactcctttgttttctgtagacgccagtggtggatcaccactccaccctcagtttaatttggacgttaccgcgattagtgcgagttgaggttatcttagtgaattattgtgcgtggcagattttcaactgtgacactgttttgtttaaaagtgcg

SEQ ID NO: 135 - DNA, 459 bp, synthetic construct
  source 1..459: mol_type = other DNA
tagggtaaatccgggagagatgcccataagggatagatgcctactgaattacaaaaaagaaataaaagaattgcgtgaaccggaacgcatcctaattgcgtcagttgaaagtcacatgtcatagcgcaagtttctacaagggattagattattttaaatatattattttggtgtttattcgtaaaatacggtaagtacagttatataaaatgtcagaaaactatttcagagacattttgttctagttttttttcttcaatttcatgcaaatgataataacattgttttataacctacaatatggtttcgttttgttgacacgaggccattttttattgtcttgcgttttgttctgtgtttaggaacattaggcatctcttcctgacaatcgggagagacgcccaatgatttttcagggtgagatgcccaataaataattgttgcttttattatattcag

SEQ ID NO: 136 - DNA, 476 bp, synthetic construct
  source 1..476: mol_type = other DNA
taggggagggtgggggaatcacggacggcgggtaatggcgaacactaaggtattcgcatgttagaaagaaatagtagatatctcgagtatcgcaatcgtgagtcggacccgaacttagtgccgaacagtaactggtgaggtgaagccgcgctcggtgttcaaggtaagttaatccttttttcttcttgtaatttcatctaattttttatataaattgtatacgtgtataatactctatgcaaaaaatagtaatctgatatttttgttgtgaagtaacgactaaatatataacttatttaaataatcaccacgtgcattgcaagtttgtaacctcaaaaaattaaatagttttaggtttctgtgtgtatcgggtattggcggacgaattgagtggggcaatctcggacgtccgtgattaccccgcttactattataggtccagtttaacaatttttaaaacgtcaaacatatttcag

SEQ ID NO: 137 - DNA, 498 bp, synthetic construct
  source 1..498: mol_type = other DNA
taggggagagcggggagggttcggacagtgggagactctggacattgctagatttcatacaaggagaaggatagttggcagccctgctgcttgtttcggtgagttttcagtcgcttttgaatggccgacgccaacagtgaggctctgacaatctgcattgttatcccgaccatttttctgattttgtgtattctgttgtaattttcggcgctccattcggaaggtgtggacttggacaatattcatttgtcaagaagcaaatattttttgtggaaactaaggtaagtaataaacataataaaccagaattgattcctctacttccattttagtcacgttgaaatgtatgtttttcttatgatggctgtatgggagagatcggacagtctgtccgaagtctccccccttatttctgaaattttttacttctatctaatctctccccgtttctgttatataagtttggttttagagaatgtgtttgttttaggttgtaaa

SEQ ID NO: 138 - DNA, 136 bp, synthetic construct
  source 1..136: mol_type = other DNA
tacactatattgccaaaagtattcgctcacccatccaaataatcagaatcaggtgttccaatcacttccatggccacaggtgtataaaatcaagcacctaggcatgcagactgtttttacaaacatttgtgaaaga

SEQ ID NO: 139 - DNA, 414 bp, synthetic construct
  source 1..414: mol_type = other DNA
tacactaccgttcaaaagtttgggatcacattgaaatgtccatatttttgaaggaaaagcactgtacttttcaatgaagataactttaaactagtcttaactttaaagaaatacactctatacattgctaatgtggtaaatgactattctagctgcaaatgtctggtttttggtgcaatatctacataggtgtatagaggcccatttcaagcaactatcactccagtgttctaatggtacaatgtgtttgctcattggctcagaaggctaattgatgattagaaaacccttgtgcaatcatgttcacacatctgaaaacagtttagctcgttacagaagctacaaaactgaccttcctttgagcagattgagtttctggagcatcacatttgtggggtcaattaaacgctcaaa

SEQ ID NO: 140 - DNA, 307 bp, synthetic construct
  source 1..307: mol_type = other DNA
tacagtggtggaaaaaagttttcggacaccccatgcatttgtgaaatattgcattaagaatcactcttaggtcttcaagtgcaatttcttttagtacagtcacagccaaaatactaaataaatcctaaaaaagccattaaaaacttaaaattgattggttccataaaaatacataagaaattttgagtattgggtcattttggtaccagtgatgaaggtcgttctttttattaaaagacacaatttttgttgccaagcttcgtgtctacataaagccagcacatttgaaagttcttcagacacaaaa

SEQ ID NO: 141 - DNA, 381 bp, synthetic construct
  source 1..381: mol_type = other DNA
tacagtcatgtgaaaaaattaggacaccctttgaaagcatgtggttttttgtaacatttttaataaaaggttatttcatctccgtttcaacaatacagagagattaaagtaatccaactaaacaaagaaaactgaagaaaagtcttttcaagatcttctgtaaatgtcattctacaaaaatgcctattctaactgaggaaaaagataggacaccctcacatgtattccctcttaaattggctcagatctcacacaggtatatcacaccaggtgcacataattagtagatcgttactctgcatgttgaatgaggcttgccctatttaaacctcagacatttagtttggtgtgctcctgactgttgaagtgagagtgagcacc

SEQ ID NO: 142 - DNA, 339 bp, synthetic construct
  source 1..339: mol_type = other DNA
tacagtattgttcaaaataatagcagtacaatgtgactaaccagaataatcaaggtttttcgtatatttttttattgctacgtggcaaacaagttaccagtaggttcagtagattctcagaaaacaaatgagacccagcattcatgatatgcacgctcttaaggctgtgcaattgggcaattagttgaattagttgaaaggggtgtgttcaaaaaaatagcagtgtggcattcaatcactgaggtcatcaattttgtgaagaaacaggtgtgaatcaggtggcccctatttaaggatgaagccaacacttgttgaacatgcatttgaaagctgaggaaa

SEQ ID NO: 143 - DNA, 380 bp, synthetic construct
  source 1..380: mol_type = other DNA
tacaacccctggcaaaaattatggaatcaccggcctcggaggatgttcattcagttgtttaattttgtagaaaaaaagcagatcacagacatgacacaaaactaaagtcatttcaaatggcaactttctggctttaagaaacactataagaaatcaggaaaaaaaattgtggcagtcagtaacggttacttttttagaccaagcagagggaaaaaaatatggactcactcaattctgaggaataaattatggaatcaccctgtaaattttcatccccaaaactaacacctgcatcaaatcagatctgctcgttagtctgcatctaaaaaggagtgatcacaccttggagagctgttgcaccaagtggactgacatgaatc

SEQ ID NO: 144 - DNA, 415 bp, synthetic construct
  source 1..415: mol_type = other DNA
tacagtacaggccaaaagtttggacacaccttctcattcaatgcgttttctttattttcatgactatttacattgtagattctcactgaaggcatcaaaactatgaatgaacacatgtggagttatgtacttaacaaaaaaaggtgaaataactgaaaacatgttttatattctagtttcttcaaaatagccaccctttgctctgattactgctttgcacactcttggcattctctccatgagcttcaagaggtagtcacctgaaatggttttcacttcacaggtgtgccttatcagggttaattagtggaatttcttgctttatcaatggggttgggaccatcagttgtgttgtgcagaagtcaggttaatacacagccgacagccctattggacaactgttaaaattcatatt

SEQ ID NO: 145 - DNA, 379 bp, synthetic construct
  source 1..379: mol_type = other DNA
tacagcgggtgaaataagtattgaacacgtcaccatttttctcagtaaatatatttccaaaggtgctattgacatgaaattttcaccagatgttggtaacaacccaagtaatccatacatacaaagaaaccaaaacaaataagttcagaaattaagttatgtgtaataatgtgaaatgacacagggaaaaagtattgaacacatgaagaaagggaggtgcaaaaaggcatggaaagccaagacaacagctgaaaactatcagtaattaaaaagcaatccagccccctgtcagtgcaaattaatatcagctggttcagtcccaactgatggcctacaaaaaggtctcattgccaaggtgtcacacaagacacatctcatg

SEQ ID NO: 146 - DNA, 367 bp, synthetic construct
  source 1..367: mol_type = other DNA
tacagtgcctacaagtagtattcaaccccctgcagatttagctggtttgataagatgcaaataagttagagccttcaaacttcaaacaagagcaggatttattaacagatgcataaatcttacaaaccaaaaagttatgttgctcagttaaattttaataaattttaaacataaaagtgtgggtcaattattattcaacccctcaaaccaccagaattctgtttggttcccctaaagtattaagaagtagttcaggcacaaagaacaatgagcttcacatgtttggattaattatctgtttttccagccttttctgactatttaagaccctccccaaacttgtgaacagcactcatacatggtcaac

SEQ ID NO: 147 - DNA, 377 bp, synthetic construct
  source 1..377: mol_type = other DNA
tacagttgaaaccagaagtttacatacactatataaaaaggcacataaacttttttttctcaccgtctaacattaaatcagattaaacttttcctgtttttggtcaattaggattaccaaaattatttctatttgctaaatgccagaataatgagaaaaggatttttttagacaatttttattactttcttcaaagtcagaagtttacatacactaagattactatgcctttaaacaatttgggaaagcccagatgatgatgtcatgtctttggaagcctctgataggtttattgacaacatttgagttaattagaggcacacctgtggatgtattttaaggcacacctgaaacacactgcttctttgtgtaacatc

SEQ ID NO: 148 - DNA, 312 bp, synthetic construct
  source 1..312: mol_type = other DNA
tacagtggcgtgcaaaagttcccggccagatggtttttgctatttttcttccaaaaaaaaggcgttaagaaattttgattagaatcatatataagggcatgttacgtgaaaaaatactatagaaaaaatcaatgctgctgattcttcatacatgtttatttaaagaataagtaaacttgcgtgttcaaaagttcccggccagtttaagctgggattgtatttaagtctgtaaaaggtcagtaagtgtttacattcaaataatattacgtagttggtagtcttcagttggtagtcagtcgctgaaatgtaaac

SEQ ID NO: 149 - DNA, 304 bp, synthetic construct
  source 1..304: mol_type = other DNA
tacagtggcgtgcaaaagtttccggccagtgacattttgtactctaatttatttaaaaaaagcctaatgaaattttaattattattaggtgcgagtataacatataggaaacaaaataatatagtgaaagaatattgtttatgttcgtgaaagtatttatagttataattgttatatttacatgtgcaaaagtttccggccacataatatatttgacctgcaataggacttcaaagagttgtttatgaatagttttgtgttgaatgtttatacttagttcataggaaatcgtcgaagcataaac

SEQ ID NO: 150 - DNA, 382 bp, synthetic construct
  source 1..382: mol_type = other DNA
tacagtggaccacaaaagtgatggtacaatagacaatttatatataaattttatttataatttttatatgaaataaaaatcaatttttacttttatgtatttattttttccaatgcccaacatcataacataaacctatttgcataatatatctaacacacaataaacaaaaaaataaaataaaattttctataaaaatttaatccacaaaagtcaaggtactcttaatttgtgtaaatcaaaatatgcaagtatcttttaaatttattgttattttgttatttgctaataagaacattcctctaatactattctggcattcgttttcagtattcactagaatatttcttcttaactttgactggtaaatacagtgataatc

SEQ ID NO: 151 - DNA, 373 bp, synthetic construct
  source 1..373: mol_type = other DNA
tacagtggctctcagcttatttcaaccagcctttttttttaagtaaaatccacataatttccaaacgaaatatgttattgcaaaaatttaaatgagagtttgttaaatatgtggcaagcaaaaataaaagtatttttttccagtcttaagcttaaacaatttaacaattttaaaaacttacaaaataaaaacaaaaaacatagtcacagcttatttcaaccagctacttttcaaatcggtttaattggcatcacttttcgggtaacggtaaatttccaagcaaatttttgttttagttatgtttccaattttttatgtaagctttttgttaaattttggcagttagttgagcaggtcacgaatttttaaaata

SEQ ID NO: 152 - DNA, 366 bp, synthetic construct
  source 1..366: mol_type = other DNA
tacagtgactcaaaaaaccgtctcaatgttatgctagtgttaacatatataagtacaacaacaaaactacagtctcaatcattctgacattttcaccatttgcttattgctcttttgtttataattacaattcaatatatttgttgtaatatttaccgttatctaaaaataagcaaaacaagtttaataagtcattgggatggttttttgagtttaattacttgaggaagctacacgtgttttttcgttaaaaaaaagtgtaaaattttaggaatttatttttcttggctgaatagtataattaaataagtaagacaaacttaaatttattgctttattcttttttaacaaatttttaagtttaat

SEQ ID NO: 153 - DNA, 354 bp, synthetic construct
  source 1..354: mol_type = other DNA
tacagtgcttcacaaaattattcgtacacttccattttacaagtataaatcaaaataaatactgcatttgaatgatatctcatttacaatatcaatcaatttactttccttttacattacaattaaataataatgataaaatttaaaaagttataagatcaacatgcttaactttgataaagacaaaatatttgcaagaatgtacacacaaaattattcgtacagctacgcataatagatattaactgcgcataatggatataaactgcgcataatggttcaaatgttacaatatatctctcattctgaggtagtactagttgccttacacatatttacgtatactctaagaaa

SEQ ID NO: 154 - DNA, 359 bp, synthetic construct
  source 1..359: mol_type = other DNA
tacagtgactctcagaaatattcggacaccagtgtatggcaagctttacgacttaatatctcaggtatttaaaaaggaatcgtctccgttcctttttctaatcatgagcgttctattcatgttactaaaaacatatgattagtttataaaatgtacatactttatataatagacatgaataaaggaaataataccacattttcgggtcacaaaaatattcggacacttcgtacgcttcgcgactccgtttttctcagaaaccgctgtttcggagaatggtaagtgtttgtatacaaacatagctttggtcgttgataccgtttcaataagcttcagtaccaactctaatattcatgaaa

SEQ ID NO: 155 - DNA, 313 bp, synthetic construct
  source 1..313: mol_type = other DNA
tacagtggcaaacaaaagtatggaaacatttttaaacaattcgtatttttgcaaaaataaagtctgaaaactacgaaatagacatgaaaacagatgtaacaaagaaagttacttttcaaaatcatatttcttttattttagagctattcattactgttttcatccaaaaaaaattttttcttttgatatagttttttatttttgaacaaaagtatggaaacatttcaaattttcgtaaaacatagagtgcgtatgtattttgtttcttttaaaatgttgaacttagttgtcatggtgataattaaacatcaaa

SEQ ID NO: 156 - DNA, 385 bp, synthetic construct
  source 1..385: mol_type = other DNA
tacagtggggagcaaaattgagtacatgtttgcatatttcacatttcggagcgaatgataaagaaaacagtaaagcaaatggaaaaaaagtttattctctttattcaatatataattgactatacattaacgagaaaagatcaacgaagaggtgcacaaagaagaaaagaaaacttgaaaacaaaaacaactcgtatgtactcatttatgcacctttgacattttaaacttcttatctgcaaaagaatcaaaaatttaccgttaattgtcatcttattgcttttgctttgtttcttagacttattgtaatgtgcttattatgatttaaagtgtcaacgtggttacattgaaagtgaagagcaatttttattattatttcaaaatt

SEQ ID NO: 157 - DNA, 377 bp, synthetic construct
  source 1..377: mol_type = other DNA
tacagtgagtaactttaatattcggacactatgagatttttggtttgttacatcataatttcgtttctaataaaacaaacaaaggacaaaatcaagtattgtattaaggtatatgtctagtaagcaatagtataaattttatttacataacttgcaacagttgttaaataacacgatttaaataaaacattgtccgattttcacgttactttaatattcggacacttggtagtgtagcaccttttgtaagtgtatatgccgagttgcttcgagctagaacgcaatgtttgtttacaaacatggtcgactgttcggatgatactaacgaatattatttcatcagttactgttcgataatcgttaagttcacgtttata

SEQ ID NO: 158 - DNA, 365 bp, synthetic construct
  source 1..365: mol_type = other DNA
tacacttgctcgcagaactctgcgtacacttgcaaacaaacccctatttctaagtttacatgtatattgtatttaatatagaagacttaatgcaatacttctttttgtataacaattttcatttatttattaacataattatgttttcattttattattaaaactaaacctaatatagaaaaaaattataaacaaaacacattaccaaaaaatgacacctcaaaagtctgcgtacattttaagtttgctgcgtacagcgctggaattcacgatcaccgttagtttttgcatgtttctttaattcctcttcgctcatttgcgtaatttgctatcactttgaaaagaaatttggcacgcgtttcatc

SEQ ID NO: 159 - DNA, 218 bp, synthetic construct
  source 1..218: mol_type = other DNA
actagtttattgtaatgtctactttaaatctctaataaagcaacactttttttctcgtttattgtttccttcaagtaggagtttattttctaaggttttctatactttatggatgtgacatttactgtatacagtaatttcattttaacgcggtccccgctataacgcggtacttggcatggatcccaaatcccgcgttctagcgagggtccggtgta

SEQ ID NO: 160 - DNA, 303 bp, synthetic construct
  source 1..303: mol_type = other DNA
agcgacggcagaaacaaggaacattggacaaatttataattaaaaaggattaattatgaatgtacatatttatctcaattaatatatgtaatttatattatattacatcatatgtacatatgtatataattttttcgaaataaatgttataaaaaaaaattttatcaatattttttttgcatggaaccaattatccccttttattttaatttaaatgaaaaaaattgtttcgcttaacgagtatttcgcttaacgagtaaggttctggaacgaattatgctcgttaagcgaggttcgactgta

SEQ ID NO: 161 - DNA, 249 bp, synthetic construct
  source 1..249: mol_type = other DNA
ttattgtacgggtattgttgattgattgaaagattttggtctatatatatatatttttttctcaatgaaatccagtaattattactttatatttttattgggcccttaaggattcaattttttttattacttaatgcatttagcgaggttattactttagtccattggcccaagtcgggatcgaaagaaattattatataatcgaggttattactttatcgaacattactttatcgaggtttaactgta

SEQ ID NO: 162 - DNA, 594 bp, synthetic construct
  source 1..594: mol_type = other DNA
ctgcgccactgcattgtgcattcacagtgccagacatgccactccggtgacagacggtgacactccgaaataaatattaatgtgagtcaatactgttaaacagtccgttttgttgttttacatttatatttatgctataaaagtcagtgtaatgcactttcacatgttatgtttgttgcagtttatttgagagatttttaaaatttgtatctttggatctttaccttaagaggtatatagctccgctaacttacactttgtgtaaagttgtaacatgctgtacagtattaataattgccttgaataaaagcaccagctaaatgattgggtaataataataataataataataataatagcgtgttgctcagttagaatgttggtatggttttgttccatgttgaatatgtttaaagtctaaagaatttagttacggtaaatgagacgtttctcaattgaataaaaaaagccggttgtttaaaacataacggtcgatttcatccattctttgcaattccgcttgtaagaatcaaccgcttataagaatcaaatcatccgggacggatgtgattcttataaaaggagtgcactgta

SEQ ID NO: 163 - DNA, 150 bp, synthetic construct
  source 1..150: mol_type = other DNA
taataataaatggtattgatataattttgttttttgatatctaactaatatctcaaataatatgactactcgaatagtatgaccaaaacctgacattttttagtcatactattaaataatatgagtcatactattcggtaatatccgtta

SEQ ID NO: 164 - DNA, 137 bp, synthetic construct
  source 1..137: mol_type = other DNA
atatgcataaaagtagtataaatacaactacctgtactgtaatactttcttttatatcggttgtataagacgacccacaattttcaagggtgattttttgactttaagggtcgtcttatacgccgaaatatacggta

SEQ ID NO: 165 - DNA, 292 bp, synthetic construct
  source 1..292: mol_type = other DNA
ctttcttggtaggctactgtttactgctaattttttattttttgttacaagccgtgtttcgttaaagcctatttatttttgttacaagccgtgtttcgttaaagcctatttatttttgttacaagccgtgtttcgttaaagcctgtgtaaagttaatttgtttcaatgtaccggtaggcacctgcggcttatagacatgtgcggcttatttatgttcaaaataatatatatttttaaattcagtgggtgcggcttatattcaggtgcgctcaatagtccggaaattacggta

SEQ ID NO: 166 - DNA, 224 bp, synthetic construct
  source 1..224: mol_type = other DNA
ttaaaaaaaataataatgtgagtgtattgttaaatagtacaataaagttcaactaaactatgttaactgttttgcttccattaccttttttttgcagacagtgtattttagcgtgcgccttataatacggtgcgccttatgtatgggttaagtacagaaatagaccccgtaattgagactgcgtcttataatccggtgcgctttatagtgcggaaaatacggta

SEQ ID NO: 167 - DNA, 606 bp, synthetic construct
  source 1..606: mol_type = other DNA
aatttatacactacatgatgtttcaatgtttgccttccggtgttgaattaattcaaatgaaatatatttacatgtatataacttatagcttttttgttaaattgttgataaattaaatgaaaatgatacttttttgtttttctcttcattactcaaataaacgaaacatcgaaataaagaacaaggataatttgatatatgagcgcaattcttttttttcagaaatccaaaatgtttttttttattccattatttttaaaacaattaaattacaatgcaataccataatacgtatatctgtgcatagatttttaaaatattacgcataattattaattttctatcaatgtccgaactaaccgactgaccgtccgaatttaccgacttgctgtccgaatttaccgacctctgttacgtcttggtttttattttatattcttttgttaacatgataacatcattcggaactctagatttttcgtaagtattgttgttttcacaatcgatgcacattcactcacacgatcgtgtcaaggtgctcaaactccaacctaatctgatttccgaagagcaccagactgtccgaagataccggcctttacccta

SEQ ID NO: 168 - DNA, 414 bp, synthetic construct
  source 1..414: mol_type = other DNA
gtcttattaggtccaacattgtggagtattgattccaagtaacattgtttaatcttttgtaaagtctatgatatgaaaaaatgtttgtgttacaattttttatagcaatgctatataacacttaagcatatgagtatagtaaaacaatatcgttcatattaaatacagtgcccaaacctccccataggggtgcccaaacctcccaaccacggggaggtttgggcactttggacacgtgttagaaaatcttttgtaactttgcaactaattataatactaaaactcttcttagcaataattaatgccaaatatgtgtgctagatatatttgaaataaaattttgattatgataaaaaataacagatttattagcgtttatgtaaaaaagtgcccaatgctcccctacctccccta

SEQ ID NO: 169 - DNA, 220 bp, synthetic construct
  source 1..220: mol_type = other DNA
ctcaggggggctgaatacttttgcacgccacacttttcagttttttatttgtaaaaaaatttgaaaaccatgtatcattttccttccacttcacaattatgcaccactttgtgttggtctatcacataaaatcccaataaaatacatttacgtttgtggttgtaacgtgacaaaatgtggaaaagttcaaggggtatgaaaacttttgcaagccactgta

SEQ ID NO: 170 - DNA, 220 bp, synthetic construct
  source 1..220: mol_type = other DNA
gtttagggggcaaacactttttcacacagggccatgtagttttggattttgttttcccttaataataaaaaccttcatttaaaaactgcatgttgtgttcacttgtgttatcttttactaatatttaaatttgtttgatgatctgaaacattaaagtgtgacaaacatgcaaaaaaataagaaatcaggaagggggccaacactttttcacaccactgta

SEQ ID NO: 171 - DNA, 221 bp, synthetic construct
  source 1..221: mol_type = other DNA
gtttagggggcaattactttttcacatagggccatgaaggttcggatagcttttttcccttaataaataaaatcatcacttaaaaactgcattttgtgtttacttgggttatctttgtgtaatatttaaatttgtttgatgatctgaaacatttaagtgtgacaaatgtgcaaaaaaataagaaatcaggaagggggcaaatactttttcacagcactgta

SEQ ID NO: 172 - DNA, 221 bp, synthetic construct
  source 1..221: mol_type = other DNA
gtttagggggcaatcactttttcacacagggccatgatggtttggattttttttcacctttaataataaacaccttcatttacaaattgcattttgtgtttacttgtgttgtccttgactattgtttaaattggtttgatgttccgaaacacttaagtgtgacaaacatgcaaaggaacaggaaatgaggaagggggcaaacactttttcacaccactgta

SEQ ID NO: 173 - DNA, 222 bp, synthetic construct
  source 1..222: mol_type = other DNA
atccaagggttcacatactttttccaccctgcactgtgaatgtttacacggtgtgttcaataaagacatgaaaacgtataattgtttgtgtgttattagtttaagcagactgtgtttgtctattgttgtgacctagatgaagatcagatcaaattttatgaccaatttatgcagaaatccaggtatttccaaagggttcacatactttttcttgccactgta

SEQ ID NO: 174 - DNA, 218 bp, synthetic construct
  source 1..218: mol_type = other DNA
gtgcttttggctagagggatcaaatacttatttccctcaatgaaatacaaatgaattaaaatattttctttaaagttattttctggattttctttttgatattctgtctctccatgttagaacacatctaccattaaaagtacagaatgatcatgtctttattagtgggcaaaccaacaaaatcagcaagggatcaaatactttttactctcactgta

SEQ ID NO: 175 - DNA, 218 bp, synthetic construct
  source 1..218: mol_type = other DNA
gttctgcttttctgatgtatcaaatacttatgtcatgcaataaaatgcaaattaattacttaaaaatcatacaatgtgattttctggatttttgttttagattccgtctctcacagttgaagtgtacctatgataaaaattacagacctctacatgctttgtaagtaggaaaacctgcaaaatcggcagtgtatcaaatacttgttctccccactgta

SEQ ID NO: 176 - DNA, 218 bp, synthetic construct
  source 1..218: mol_type = other DNA
gataaacttttgttattgaccaaatacttattttccaccataatttgcaaataaattcataaaaaatcctacaatgtgattttctggatttttttttctcattttgtctgtcatagttgaagtgtacctatgatgaaaattacaggcctctctcatctttttaagtgggagaacttgcacaattggtggctgactaaatacttttttgccccactgta

SEQ ID NO: 177 - DNA, 216 bp, synthetic construct
  source 1..216: mol_type = other DNA
tgtcataattttttttggtcaaatacttttgtccctcattacttttgcttgtgttaaattttcttgttataagtgcacatttgactgttctgtttaaatattctattatatatatgttatataaacttcctgtattaattatcctaaagcagtccctaaattttttaaaatacataaaaaatagagttgtgcaaatacttatgtctaggcactgta

SEQ ID NO: 178 - DNA, 228 bp, synthetic construct
  source 1..228: mol_type = other DNA
cactgctgtatgtatatttttgacccagcagatttgatcactttttctgttaacccataataaagtcataaaagaaccaaacttcatgaatgttttttgtgacaaagaagtatctgttccaatcactctatcagagaaaaatcagacttgtagaaataacgggaaactcaagagagccacgacattatgttctttacaagtgtatgtaaacttttgaccacaactgta

SEQ ID NO: 179 - DNA, 230 bp, synthetic construct
  source 1..230: mol_type = other DNA
tgtgatttttctattggggtgcccaaatttatgcacctgtctaattttgttttgatgcatattgcacattttctgttaatccaataaacctcatttcactactgaaatattactgtgtccatcagttatttgatagatcaaaatgaaattgctgatccaaacacccaattatttataaatggaaatcatggaaattgtcaggggtgcccaaactttttcatacgactgta

SEQ ID NO: 180 - DNA, 247 bp, synthetic construct
  source 1..247: mol_type = other DNA
cactttgggcccaatttggatattttcacttaggggtgtactcacttttgttgccagcggtttagacattaatggctgtgtgttgtgttattttgaggggacagcaaatttacactgttatacaagctgtacactcactactttacattgtagcaaagtgtcatttcttcagtgttgtcacatgaaaagatatactcaaatatttacaaaaatgtgaggggtgtactcacttttgtgagatactgta

SEQ ID NO: 181 - DNA, 273 bp, synthetic construct
  source 1..273: mol_type = other DNA
gcatcattagaattaattttatttgtttgttaactactacttctaaaagtgtatgatttattttgtccagggcaaaatgtaacaatattaaataatcaatctttcacaaaaaaattgagaacaattttgattatttcttatttcctttatttaaatactttgaaatgctttaattttcctgtatttaataatatatttgcttgttaaaaaaatagttacagtaacaaattcaattttttataaggtgtatgatttattttgtccagtactgta

SEQ ID NO: 182 - DNA, 271 bp, synthetic construct
  source 1..271: mol_type = other DNA
gttaatttttttgtggatcacattttcgaagataaaattaattttaaatcgtacgcaaactttttccgcgtgcttttatattttgatggagaaattggtttttagccccaccactgataatttctttgcaactttttttacgatgttagtattcgagataaatttagcatgctaccaaaaaatttggacatcatactccaaaaaatgaataaatgacaacggattgaaattcttgaaacatctcgtacgcaaactttttcctctaactgta

SEQ ID NO: 183 - DNA, 277 bp, synthetic construct
  source 1..277: mol_type = other DNA
ttgttctaaacctaaaattgaccattgttccatgttatttgtttgtaaaattgtatttagagacgatcgttctcttattttgcactgttcaatttttgttattgtttgttagattgctctaatgtattaaatatagttatgtttgtcctgatattttgtagaaatatgttaaatacattccttagggccccatcaaaagattcattaacctactctacatggttaacctttattttcaaaaaatgaaaatcgttctcttattttgcccgctagtgta

SEQ ID NO: 184 - DNA, 260 bp, synthetic construct
  source 1..260: mol_type = other DNA
ttaaggatgaaggtttttgttaaatttgttgttttgttgcgttttttgtcaagtgtcccttaattctgctcacactaaattcataattataagttaaatatacataacttttttcttatccttcggatcgtaataaattcttagcatattattaggtatcccaatacctcttttgtgttaaaatttcagcgttgaaccttttacagtttttgagatatgaaattttttatttttgtcccttaattatgctcgcaactgta

SEQ ID NO: 185 - DNA, 325 bp, synthetic construct
  source 1..325: mol_type = other DNA
ttatagtttttttttattgcatttaacattaaattacttcatataaccatagtttgtcgttatatattattttcataagttttgagcgagtttgcttgaacactcctattattttgagaaacccatattgggtgttattccctttttattttttttactaagtatgtattcaatgaagtaagttttttttagttttaagttttttaatacaagtcatatttgttaaaaaaaataaatttaaaaaatttaaatcgtattgacatagtttttttcaccgtttttcacatttgtaatgggcactcctattattttgaaaacaactgta

SEQ ID NO: 186 - DNA, 315 bp, synthetic construct
  source 1..315: mol_type = other DNA
atttttcgttctatttatttaaactttagtgttgggtagtgttatgtaaatgttgaatcaaataaaatttgagttgaaaatattttctgacaatgttgccttacttttgtcgcattaatatcgggcttttttatatttcaagctagtagtatgtcataataattacccacaggctatccattttcaacacgaataagaatatctttaaacatttattataaaattaaattgaaggtaaataccaactcttaaaatttaaaataccaatatttgtatatttttcaacatgttgccttacttttgtccgatagtgta

SEQ ID NO: 187 - DNA, 289 bp, synthetic construct
  source 1..289: mol_type = other DNA
agcttctgatgtaatcaaattaattttcggtggttaattacaggttttgtttggaagttgcaacagataatggttttgtcaaaatactttctttctacatttgtttaaaaaatgtaaaaaataacgcagttttcgtttttaaatgtgatatttgttttttctttcttttctactgagaggcattgttgcttttggacaaatattataactttgttataactttgtttcagaaataaatatttattttctataaattgtgcttgtcaaaatactttccgccgctactgta

SEQ ID NO: 188 - DNA, 178 bp, synthetic construct
  source 1..178: mol_type = other DNA
ctctttgcatcaacttcatgtaattgtcaataaaagcctttgacacttatgaaatgcttgtaattatacttcagtattccatagtaacatctgacaaaaatatctaaagacactgaagcagcaaactttgtggaaattaatatttgtgtcattctcaaaacttttggccacgactgta

SEQ ID NO: 189 - DNA, 244 bp, synthetic construct
  source 1..244: mol_type = other DNA
tattaatttaataattaaaacatctggaaacgtgcaaaattgagtttatgctggtttttttcgtttttttttgttattctcctagaatttatctctggcatgactgtttttgcttaaaatttattaagaataaaataatgaatataaaacaaaaaaaattttgggattatcttgtgtggttatgattttgtgggtgacgaaagtctgagttctgaacatacactcaattttgctcgccactgta

SEQ ID NO: 190 - DNA, 244 bp, synthetic construct
  source 1..244: mol_type = other DNA
tttaatatttccttaaatttttccattaaaggtgcaaaactgagtacatgccttctgtttttgttttttttttctatctcttacattttttcttgtattattgtagtatttaagtctatttcttacaaattaaataagtaatcgagaaaaaaaaagtttgtttcatttggttcattggtttacgttttatgagcggttcaacatgcatattctgaacatggaatcacttttgctccccactgta

SEQ ID NO: 191 - DNA, 198 bp, synthetic construct
  source 1..198: mol_type = other DNA
tgtatttgtgcaaataaacattcgttaaaccttttgttttgaattttgtctttttgatgattacaactaacacaacgactatcattatgaaaattattgaactgttcacgcaatgtattaagtatttaaaatttatttttctatctgacaaaaacttgtaaaataatacatgttgcgtttcttttttgggagagtata

SEQ ID NO: 192 - DNA, 200 bp, synthetic construct
  source 1..200: mol_type = other DNA
ctgttacttttgattttgaccccccctttgttcagggacacattattccatttctgttagtcacatgtctgtggaacttgttcagtttatgtctcagttgttgaatcttgttatgttcatacaaatatttacacatgttaagtttgctgaaaataaacgcagttgacagtgagaggacgtttctttttttgctgagttta

SEQ ID NO: 193 - DNA, 326 bp, synthetic construct
  source 1..326: mol_type = other DNA
tgttttgaacttttaaggtcaaaatgataatgataatgaacatttccagtgcaaattgtaactcttacctctatctgtaaattctgtagctccatactttttcatgtgttaaaaatgtcaaaaatgagtacctctctgccaatgtcatttccaatattaaatctcaaagcaaatctatctcaaatggtcttctttgttgttaaatgtgcgatggatccgatgcaatttgaaaaatttagttaccaagttcgatttccgcttagtgcatcatataattttgattatgatgaaaaaactgggatgtgcttaacttttttctttgtgta

SEQ ID NO: 194 - DNA, 261 bp, synthetic construct
  source 1..261: mol_type = other DNA
gaattatgtttttttgctttcattgttttctaaaatacaattttttccatatgtgctatctttatttccacccgatttgcacccacaaactattattttcgtggtaaaattttgaaaagacaacgaatacaataatgattataaggaatcggtaactagttaaagtaacattattcagttaaaatttcaactatattgatgcagtatttttaaacttattacaaaatttcaaaatgtgctatcattattgccaccacttta

SEQ ID NO: 195 - DNA, 76 bp, synthetic construct
  source 1..76: mol_type = other DNA
tgttattttcaaataacaataaatgtattctcaattttgacctccaaaggctcaatactttttagacaacctaata

SEQ ID NO: 196 - DNA, 159 bp, synthetic construct
  source 1..159: mol_type = other DNA
gagtagtgacgataagtttctgtgactgttccacattggatgcaataaatctcacattggaccttgatttgaaagccaatgatgtgatatttgcgttaaggacatgtgcggtgttcactttgcttgcaactattcccaagaaggacgggcaaaggtgta

SEQ ID NO: 197 - DNA, 255 bp, synthetic construct
  source 1..255: mol_type = other DNA
ttataaaacattttttttacaagtattgcgttacattatgtaatattagtaggtatatttagtaaattgttatattcttctttttctaataaaacattaaaaaagaggaaaataatttgcaataacgtgaaagagttttgacaaatagtgaatttcatttgattattaatgtcaaagctacataggtgggaaatacttttgataaatagtgaaatacatttgacaaaagaggaaaaccaaaagaaaaaaactgta

SEQ ID NO: 198 - DNA, 369 bp, synthetic construct
  source 1..369: mol_type = other DNA
atggaaactgcgttgctgactcgcagtgacttgcgagctctctctatcgtttgttaacttccttgtatcagactgctttaggaaaagtttaatccacttgcactgttttagttttatatgttttatgaggaactgacttatgtttattagtgaccgttttcatgctatcatgtcatgccatgatgtcataagcatattaattaaaaatttctatattgaaatcaaatctgatgttttttatttaccggtagtgtgcgttggaaaaggggtagtcttatacggagagtatagcacgaattatatattttaacaaaaaagttgggggtcgtcttatacgcccagtcgtcttatacgccggaaaatacggta

SEQ ID NO: 199 - DNA, 455 bp, synthetic construct
  source 1..455: mol_type = other DNA
actggaaatgtaagtgtattgttcaatatttgatgtttaacagcttaacaatgttaatagttactgtgaatgagttggttaaattttaactcatccgattgttttattttgctgaatgcactttaaacgccttttaatccggcgtgagagggatccgggcatgcttgatgccgaaattgcccagctgtttaccgtaactcgacacggaaggtgaagagtttgagggaattgtggaagttgaatgtactggaaaagtgtattttcaatatttaatgtgttacagctaacagtgttaagagttactgtgaatgagcttaataaaggtcgagttatctgactgttttgtttgtcttaatgcgtcttataatatgatgcgccttatgtatgaaaacagagccagtcttcaacagtgcgccttataatctggtgcgccttatagtgcggtaaatacggta

SEQ ID NO: 200 - DNA, 318 bp, synthetic construct
  source 1..318: mol_type = other DNA
agtgacatgtggtttcaaatacaggaaaaatggaggtaaataaatagcggttattttctcttggttctgttacgttttaatcagcaaagttgctgccgtgttaaaaggcattgttcggaaagaatctgttcaggtacatacttgtacatttgcactacaagattgttctgtacatgcagtaaatatctattttttcatcatagatatctgcggcttatagctgggtgcggcttgtatatctttttttttttaattttttaaaaatagagcggatgcggcttatatacaggtgcgctctattgtccagaaagtacggta

SEQ ID NO: 201 - DNA, 149 bp, synthetic construct
  source 1..149: mol_type = other DNA
aaggtcagtttactattaacaaaaacctattattgtatccattcttttaaaatcatactttaaaatttagggtttttccagaaaattctattaaatttcttggttgcgtcttatacataggagcgtcttatacaccggcaaatacggta

SEQ ID NO: 202 - DNA, 356 bp, synthetic construct
  source 1..356: mol_type = other DNA
cacggatggtttggtaaacttgttagcatgttatttatgctatagttatctgaataactcttaatatgttatgttaacataccaggcacgttctcagttcgttgtttatgtgtcatgtaacgttagcataccgtacaattattcagcctgttgttgcctctattctatttttattttaaattgcctttcaagatgacatgtctgttcttggtgttggattttatcaaataaatttcccccaaaaatgcgacttatactccagtgcgacttttatatgtattttccttctttattatgcattttatggctggtgcgacttgtactccggagcgacttaaagtccagaaaatacggta

SEQ ID NO: 203 - DNA, 352 bp, synthetic construct
  source 1..352: mol_type = other DNA
atactgtaccggtaacgtaagtttttagagtacagtacgtacatgtatagttgatatttcattgttttcttaataaaatattttaataaagtatttttcacttgaactggcgtggttttgttttttctcttttaaaatcatttcacattaagtactgcgcagcaatcatcacaattaggacttacaatggcgccgttataacatgcaaggcagtgaattgcgatgttgccacgccaaacactgtctaggtgctaagttgtagcggcgctattattatttctgttatccgtctattcggttatccgtcatggtcccggtcccgagggtgacggttaatcgaggttctactgta

SEQ ID NO: 204 - DNA, 180 bp, synthetic construct
  source 1..180: mol_type = other DNA
ttaatagtatgtatgtattgcataaagtgttcttagaattaaagtgattgattaaaatgattcgtatttttttgtattaaattttgactatgtttaatcataaacctcgatatacgcgaatttcagttaacgcgattttttccggaacgtatccctcgcgtaaagcggggtattactgta

SEQ ID NO: 205 - DNA, 182 bp, synthetic construct
  source 1..182: mol_type = other DNA
atatgtacatcatattatgtataagttgttaatacaaaatttctaacaattaagttgttaatttactttaatatgtatgttgaattcagtaatacaataattaacctccgtattatccgaactttcagttatccggaaaggcctgggtccacattatttcggataaccgggcggctactgta

SEQ ID NO: 206 - DNA, 166 bp, synthetic construct
  source 1..166: mol_type = other DNA
cgttcaaataacattcttttttatgcacataactactaacaatagtcagaaataaactttttatttttctaattctgatgtttcttctctccatttaacgacaactctctataacgccataaaatgcacagtcccttcagtgtcgctatatagagtttacactgta

SEQ ID NO: 207 - DNA, 454 bp, synthetic construct
  source 1..454: mol_type = other DNA
tccttatttattatatgtacatactacatatgtataagtttggtttccatttatatttaccgtattttggcttcagatttgttttcattttactatacagtacatggttttcaaacagtttattgtttgttttgatacttgagtgatttaaatataaacgagtatgtatttttaaaattgctacatacatgtttttttttttttttttctttttaacctttttcctacattgaaaaaataactttagtgtaacggtaacaatgattcgtattatagaggttggccataaaaatcttcgttttatagaggttatctcagagcgaggagaaacctctataagacgaataacggcagtattaaacctcgataaaacgaacctctttattaagaatacctcggtagtacgaatttttcccttaccccttgaaattcgtcttatagaggttcgactgta

SEQ ID NO: 208 - DNA, 177 bp, synthetic construct
  source 1..177: mol_type = other DNA
tattgttcttttatattatacgtttgtattgaagtaaacaaaatgcggtttaatttcctacatgaatatacatattatctttcctattaatacctgtcaccggttgttacaactatcggtttttacgactcaatatgagtagtcccttcgatgtcgttataacagattttgactgta

SEQ ID NO: 209 - DNA, 250 bp, synthetic construct
  source 1..250: mol_type = other DNA
agtaagatttttattataaaatatttaattgtgaaaacgtatcatttttttattaatgtttttttattatactattccaatgtacgagtagatttcaataaacagtgttgtactgtatatagcagaacatttgcgtttttactttgtcctttcactcagatttaaggctgctttttttataacgtcactcggatataacgttaaaaaacttccggtccctttgatgacgttataaccaagttccactgta

SEQ ID NO: 210 - DNA, 366 bp, synthetic construct
  source 1..366: mol_type = other DNA
aataaggttatcattttgtaactttttaaagtcaattgtacatatgtatataagaatacctatgtgtaaaatacatatgtatacattgtaagtttttttttcaaaaattcttatgtaataaagttttttttgtaacaactgaattatttttttgtgccaaaaatctaaaactgaagatcatttattaacacttattgtgtgtttgtactacatgctcgtacttaattttaattactttgcacgtttccaaaataaaggcattttacattggtatctgtattttattgttcccaatagtggccacttcccttaacggccacattcctttgaaaatttttgtggccgctatatggaagttttactgta

SEQ ID NO: 211 - DNA, 170 bp, synthetic construct
  source 1..170: mol_type = other DNA
tttatatgtataagcatttattatctaagacatacatacaattaaaaattcttgtattaaaaattcttattaatttggtttttttttgttcactccatataacgataactctctataacgacaaaaaatgctcagtccctcgagtttcgttataaagagtttacactgta

SEQ ID NO: 212 - DNA, 420 bp, synthetic construct
  source 1..420: mol_type = other DNA
aaaggtttctatggaatattttatgtatttttttacttttgtgcatttttgttaatttcatttgttttattctatcataaggattgtttcgttctattttattgttatatattatattttcattttgcttaatttgtaaatttgtatacatatgtacatatataacctgttttttttcaataaatatgtacataattgatactgctttatatacatatctatctatgcttttcctttcaccatttcctaaacaattaaacaaacactaaaacaatgctgagtaattttccatcagtttacacaatacttgtaggtgaaaaagtggggataattaaggtaacctctgattaagggacacctcccattaacggacagttttgagtgcaccgagggtgtccactaatgggaggttttactgta

SEQ ID NO: 213 - DNA, 504 bp, synthetic construct
  source 1..504: mol_type = other DNA
caacagtaaataagttttgcaaactctttataacatgtacgtaagcctacattacagtagcattttaaacatttaaaatacagtacagtagaaccgtatttcagacttgtgtatttatactgtacaacataactttttaagacgttacagtatttcatattcatatttatgtaataatattactctatacgtttagttaaaaaataaataaatttaaattgatgtatggtgtacttgttataatttcttattcacatttcccgataaagtaatgtacgattcttaattgtactgtattattgtagtgggtgtggacctctatatatcgaagtctgtcagagttaaccttgatataatgaataatacaacaacagttgtaaacctctatatatcgaagtctgccccaaacagccttgatatatcgaacctcattatatcgaaattcttgatatatcgaagttttgtaaatcctcttgaggttcgatatatcaaggtcctactgta

SEQ ID NO: 214 - DNA, 263 bp, synthetic construct
  source 1..263: mol_type = other DNA
ttttgttttaagtacgcatgggcatctatccctggtatctataggcaactcacccataggcctgggagagatgcccattttctatatttttttctagctgtgttatatcctaaaatatgcgtttaatatttgttaattttatgtaagacagattaataaagtctaatttaagtaaaatggcgtttttttcttctgtgtcataaatgtataattaaatatgatgtttttttaaaactataggcatctctccctgacttacccta

SEQ ID NO: 215 - DNA, 250 bp, synthetic construct
  source 1..250: mol_type = other DNA
gctatgttcgtgattaccccatacgccatccgcaattaccccacatgctgcgggtaattccggacaccaagtgttttgttttttcctcaaaatttgattttatactaaatatttatcaaagttgattatgagagtacaaaaatgattagtttaagataacatttatcaataaaagacttttataactttatttctttttgttttattacagttctcccttaggtgttcgcaattaccccaccttccccta

SEQ ID NO: 216 - DNA, 356 bp, synthetic construct
  source 1..356: mol_type = other DNA
taacatcttttttgtcttaaaataaatctcttgctaagtaatttttaagttttgtggtgcttattttgttatttttactaataaatgcaactttttaattctgtgttagtgtgtccagaatctccccctaacttgtccattgtctcccacatagtgggagatttcggacaattttcaagtttgaaaaaaaaattatttgtgtaaaaactaaaatcatttttgtcaaagtctactgaaattatgaagtcttaatccctggagattgaaaataattaaaatttggaaatatcaattttaaattaaagctacaattcgtagttaaaaaaaactgtccgaaccctccccgctctccccta

SEQ ID NO: 217 - DNA, 84 bp, synthetic construct
  source 1..84: mol_type = other DNA
accctatggattaggaatgggatgtcacttaagttcatatgcgagtcaaggcaggtgagcgaatacttttggcaatatagtgta

SEQ ID NO: 218 - DNA, 164 bp, synthetic construct
  source 1..164: mol_type = other DNA
tgtaaaaaaaatcttatttcaaatacaaatcattatttctaaccttgtcaatgtcttgactctattttctattcatttcacaacatatggtggtgaataagtgtgacttttcatggaaaacacaaaattgtttgggtgatcccaaacttttgaacggtagtgta

SEQ ID NO: 219 - DNA, 297 bp, synthetic construct
  source 1..297: mol_type = other DNA
tttgatgatgtgatggtttatttattttttgttcagttttgaacacattctctgttatttgttgactttgataccgacaatgttgagaactgacatattgaaactgtcaagaatttagttttgttagtttttcttgtaaacaataaacaaaaaaatataatttgtatttgtttgtatctgtctaatgcagccacaccttttgaaacacaaaaaagatttttccacaaatatttcatgataatatttgagattgtgtaaaattttaagggtgtccgaaaacttttttccaccactgta

SEQ ID NO: 220 - DNA, 221 bp, synthetic construct
  source 1..221: mol_type = other DNA
gggcaagggtgtcctatctttttcctcagttagaataggcatttttgtagaatgacatttacagaagatcttgaaaagacttttcttcagttttctttgtttagttggattactttaatctctctgtattgttgaaacggagatgaaataaccttttattaaaaatgttacaaaaaaccacatgctttcaaagggtgtcctaattttttcacatgactgta

SEQ ID NO: 221 - DNA, 299 bp, synthetic construct
  source 1..299: mol_type = other DNA
tttagtgattcacaggattgctaaatcccagaaaaaaaaaatgtttgtacaaaatagttttgagtttgtacagtcaaaggtagacactgctatttttttgaacacacccctttcaactaattgcccaattgcacagccttaagagcgtgcatatcatgaatgctgggtcttgtttgttttctgacaatctactgaacctactggtaacttgtttgccacgtagcaataaaaaatatactaaaaaccttgattattctggttagtcacattgtactgctattattttgaacaatactgta

SEQ ID NO: 222 - DNA, 287 bp, synthetic construct
  source 1..287: mol_type = other DNA
tgatgtgttggagcgttcttttgtttttcatgattccataattttttcctcagaattgagtgattccatatttttttccctctgcttggtctaaaaaagtaaccgttactgactgccacaattttttttcctgatttcttatagtgtttcttaaagccagaaagttgccatttgaaatgactttagttttgtgtcatgtctgtgatctgctttttttctacaaaattaaacaactgaatgaacatcctccgaggccggtgattccataatttttgccaggggttgta

SEQ ID NO: 223 - DNA, 165 bp, synthetic construct
  source 1..165: mol_type = other DNA
aacatgttttcagttatttcacctttttttgttaagtacataactccacatgtgttcattcatagttttgatgccttcagtgagaatctacaatgtaaatagtcatgaaaataaagaaaacgcattgaatgagaaggtgtgtccaaacttttggcctgtactgta

SEQ ID NO: 224 - DNA, 218 bp, synthetic construct
  source 1..218: mol_type = other DNA
ataaatatcagtaagtgtgttcaatactttttccctgtgtcatttcacattattacacataacttaatttctgaacttatttgttttggtttctttgtatgtatggattacttgggttgttaccaacatctggtgaaaatgtcatgtcaatagcacctttggaaatatatttactgagaaaaatggtgacgtgttcaatacttatttcacccgctgta

SEQ ID NO: 225 - DNA, 207 bp, synthetic construct
  source 1..207: mol_type = other DNA
acctaggggttgaataataattgacccacacttttatgtttaaaatttattaaaatttaactgagcaacataactttttggtttgtaagatttatgcatctgttaataaatcctgctcttgtttgaagtttgaaggctctaacttatttgcatcttatcaaaccagctaaatctgcagggggttgaatactacttgtaggcactgta

SEQ ID NO: 226 - DNA, 219 bp, synthetic construct
  source 1..219: mol_type = other DNA
tgaaatgtatgtaaacttctgactctcaagaaaataataaaaaattgtctaaaaaatgatctctctcattattctggcatttagcaaatagaaataattttggtaatcctaattgaccaaaaacaggaaaagtttaatctgatttaatgttagacggtgagaaaaaaaagtttatgtgcctttttatatagtgtatgtaaacttctggtttcaactgta

SEQ ID NO: 227 - DNA, 276 bp, synthetic construct
  source 1..276: mol_type = other DNA
ttataacatattaaataatgtactgcaaaattgttacacaatgaaaaaaaaagcataatgtttcgttaaacatactggccgggaacttttgaacacgcaagtttacttattctttaaataaacaagtaagaaaaataattagcgttgatattttttgtaatattctttcatgtaatacgaccttatttatgattctaataaaaatttcttaacgctctttttttggaagaaaaatagcaaaaactatctggccgggaacttttgcacgccactgta

SEQ ID NO: 228 - DNA, 277 bp, synthetic construct
  source 1..277: mol_type = other DNA
cactaaaatataatttaaactagctagagtgtattatagaatatgtggactaataatttttctcgtagtacaatgtggccggaaacttttgcacatgtaaatataacaattataataataaatatttcctcgaacataaacaatattctttcactatattattttgtttcctatatgttatactcgcacttgataatggttaaaaagtcattaggtttttttttaataaattagagtacgaaatgtcattggccggaaacttttgcacgccactgta

SEQ ID NO: 229 - DNA, 251 bp, synthetic construct
  source 1..251: mol_type = other DNA
cagaactttattagcccatagtctgaaatgtaccttgacttttgtggattaaatttttgtaagaaattttattttatttttttatttattgtatgttagatatattatggaaataggtttatgttatgatgtagggcattagaaaaaataaatacataaaagtaaaaattgatttttatttcataaaaaaattataaataaaatttgtattgtaattctctattgtaccatgacttttctgggccactgta

SEQ ID NO: 230 - DNA, 263 bp, synthetic construct
  source 1..263: mol_type = other DNA
aattacaaaaaaaattaaaaaattaatagttgaagtcactggttgaaataagctgtgtcccctgaagtacctgtttttttaatttttagtttttaagtttttaaaaatgtgaatttcagacctaataaaagctttacttttcatataaacaaaatttatattaaacccaatttaaatttttccaatacgatcttttgtttagaacttacgtaagctttattgtttttaaccagggtggttgaaataagctgtgatgcactgta

SEQ ID NO: 231 - DNA, 249 bp, synthetic construct
  source 1..249: mol_type = other DNA
gcctttataaattcaaaaaattagttaaataaagtaaactcaaaaaactatcccaatgacttctaaatttgttttgcttatttttagataacggtacatattacaacaaatatattgaattgtaattataaacaaaagagcaataagcaaatggagaaaatatcaaaaagattgagactgtagttttgttgttgtacttatatatgttaacactagcataacattgagacggttttttgagtcactgta

SEQ ID NO: 232 - DNA, 263 bp, synthetic construct
  source 1..263: mol_type = other DNA
attattataacattagtgtagttggtaaatatttatatgtgtacgaatatttttgtgtcacattttttggagtatttttcgttttgttcaaataactattacctagaaaatttatatatgtaaccaatcattgtaagttaattttaatatataaagacatactattggaacattttttgaattaaatttctttcgtaaacatattttatatcgttttatacttgtaagtcagaactgtacgaataattttgtgaggcactgta

SEQ ID NO: 233 - DNA, 275 bp, synthetic construct
  source 1..275: mol_type = other DNA
agtggggtggataatgttaaattttgtgtcaaagatgttatttttgatagtgtccgaatatttttgtgacccgaaaatgtggcattatttcctttatttatgtctattatataatgtatgtaaattttataaactaatcatatgtctttagtaacatgaataaaacgcttattattagaaaaagtaacggacacgattccttttcacataactgagatattaagtcgtaaagcttgccatacactggtgtccgaatatttctgagagtcactgta

SEQ ID NO: 234 - DNA, 288 bp, synthetic construct
  source 1..288: mol_type = other DNA
tcaaaatacagtataacttgatatctaaagtgttccattagttgtaaattgatgtaaatggtgaatgtttccatacttttgttcataagaaaaaaatatttttataaagaaaattattttttttaataacgtacctaatcatttggctcaaaacagcacagatgtaatacttgtgacttatttcatatgttacatcagtaaaaaagaaaaactttcgagttttatcagctttttgtgttgaaaaatgaaattttagaaaatgtttccatacttttgttcgccactgta

SEQ ID NO: 235 - DNA, 275 bp, synthetic construct
  source 1..275: mol_type = other DNA
ttttttactaaaaattccatttaacgatcttgtctgcagataagaagtttaaaatgtcaaaggtgcataaatgagtacatacgagttgtttttgttttcaagttttcttttcttctttgtgcacctcttcgttgatcttttctcgttaatgtatagtcaattatatattgaataaagagaataaactttttttccatttgctttactgttttctttatcattcgctccgaaatgtgaaatatgcaaacatgtactcaattttgctccccactgta

SEQ ID NO: 236 - DNA, 285 bp, synthetic construct
  source 1..285: mol_type = other DNA
ggtacctattattattaaaattagcttaaacataagcttcggaatacgtagtttctataagtgtccgaatattaaagtaacgtgaaaatcggacaatgttttatttaaatcgtgttatttaacaactgttgcaagttatgtaaataaaatttatactgttgcttactagacatataccttaatatagtacttgattttgttctttctttgttttgttagagatgaaattatgatgtaataaaccaaaaatttcatagtgtccgaatattaaagttactcactgta

SEQ ID NO: 237 - DNA, 276 bp, synthetic construct
  source 1..276: mol_type = other DNA
caactaacaaattccagcgctgtacgcagcaaacttaaaatgtacgcagacttttgaggtgtcattttttggtaatgtgttttgtttataatttttttctatattaggtttagttttaataataaaatgaaaacataattatgttaataaataaatgaaaattgttatacaaaaagaagtattgcattaagtcttctatattaaatacaatatacatgtaaacttagaaataggggtttgtttgcaagtgtacgcagagttctgcgagcaagtgta

SEQ ID NO: 238 - protein, 541 aa, synthetic construct
MPRFTLTHRQKLSIRNYFEERQAEGRPLTQQALAQWAKQNFSLPFIPSQPFISKLLSRKKRLELQDRAIRRCRKGQQPALEAVLSEWVHSRQLARPCITGDVIRQKAVRLQEEINYSRTEDSKFDMKFTVGWLSKFQKRWNLRSRRMHGESADADMESVRDCLPSLASLVQSYHPNDVWNADETGLYYQMPPDRTISHAPLPGRKKAKVRITILVCGNASGKEKFPLMIIGQARKPRAFKGKTGQELGFDYWHNRKAWMNSSLCFAWLSRFDAYIGRTTGRKVLLLLDNFSGRRTKDHLPELQNTKISFLPPNTTSVLQPMDAGIIATLKTRYRKLQYERALDLLEESNSQIYKIEQLLAMKYITGVREDMDEKIINNCWFKTGLLARETSGICDNEDHLPVRETDELIGVIQQLVVPNARMSISALLNDEDSECYDEPDIRTMASSVVHELEGGDSYSEAEIGTDADSTSPSSALSRDGQLAALKTTLDIVEAQLAPNLAVLRYLRRIKRDLAAAKVQTQTQTRIDSFFSGASNLAGQAS

SEQ ID NO: 239 - protein, 341 aa, synthetic construct
MLGSLDSEVQNWIRQVRLNGGVINLRIVMAGAEAIVTKFDRKKLARFGGHIEITKHLARSIMRRMGFVKRKGTKAVKTLPSDFDDIKCEFVKKVNNVVSEYSVPDSLIINWDQTGCQLVPGGEWTMEEKGSKQVTVAGIDDKRQITLLLGITKSGTLLPPQLIYAGKTERCLPQSVEFPSDWDITFTETHWSNESSMLRYVDKVIVPYVNEIREELPLDRINQKAIAIFDIFAAHRTNSLIEKLKSHNIQPLYVPGACTDKLQPLDVAVNYDYKEKLKAEFHEWYSTQVFESLDRDGPGSANIDLRTSTLKPLHANWVINTHYNMSKRPELIKSGFQKVGL

SEQ ID NO: 240 - protein, 622 aa, synthetic construct
MTPMDAAIEAIESLKPGDSINYTKIAKEFGVNRITLSRPHKGIQRSRRDQYEEQRILNDQQAKDLIKYIDKLSGKGLYISHEMLRNFAKELTGKKPGNHWPGRFLKRHQIELSSAYTTAMDSNRKRADSAYKYSRYFDLLAQKLDKYKVEPGNIYNMDKKGFLIGMLSKGLRIFSKRKYKQGNFKQRLQDGNREWITAIACICADGTLLSPVLIYQAASSDIQDTWLQDFDPQHHKTFFASSPSGWTNDKLGYAWLTGVFDRETKDKARRQWRLLFLDGHGSHLTMKFFDYCDDNKILLATYPPHSTHSLQPLDVGIFSPLSHAYSSELEAYLHISMGLSHITKRDFFRLFFPAWVKALSSKNIISSWRTVGIHPFNPEIVLARFSREPQSRPSTSESSRSILGAEDWRKIKKLLHDVVEDVYSENTRKLSLAMHNLSTENILLKLQCEGLQIALQNEKKKRQRGKPLQFQLKASDDGGAVFYSPQKIQQARDLQLGKERAAEQLKASKEEQKVRRQQEKEAKQRLIEDRRKIRASQREIHRLEAEQKRQEKEDARISKEAAKQLQIDFQQAKKTPRKSSKASNHTDTQDTGPPSHVVVEEVPPTVNRRGREIRLPQHFRTN

SEQ ID NO: 241 - protein, 570 aa, synthetic construct
MPLQRNMLSYRSVAPIIALPIMENYRDDLWKAIFRRFSEEVSWKTLEKSHGIPATTIRRFYKKWTRAYPESQAQFYGDIHIQCSSAGRPRMLHETEESTIAEAMKHFADHNTPLTRCQTQDLVAHYVGLLPHSRQQDIKFVNNRPCLPWVDQMAKRHGLNYKAVRVIEQNRVEALSKSVFAEHIARVQAACDRFRIKDSKLIFNIDQSGASFEKMVGRSLRKGYGAKEMPLVQTGIRTKGKLDRVTVMPVVSAAGVAYTPVIVFPGKQAHYRHVQGQIQTLRDVLLPCYLFQREIPGVNTEIIYLWAQKFDETAELRANNQHLLLVIDGYGSHVQFRTLQLLKESNIVVIALPSHTSHVLQPLDVTVFGPYKSYLQQAMRSAALRKRILNAFDIGECISQAYARAVIGPNIKSGFVKTGLWNPESLTTDVFALCSLFEGSCGEGNNGNVELHALLRSFARGGRSLLRDAQVQESGTVRIGTRTGSHLTSESVLDALKQRDERRRSRTRQLESREMNKDIYCETAKEKRWLVSLAAERSIVRGKLRQSRRERRLVRSMRAAAALQNMSDIS

SEQ ID NO: 242 - protein, 561 aa, synthetic construct
MPIPSSSPIVAFLQQAVSNAQNVAPTPDLGPSDATPTDTVPSPLVPTQAQESAPPSNRKRALQQVVTKRDRKNVVKWMDQQLTDGTPIKGLMARTVRHFPALFSASEKANIIKAMRWWKDKEVILHEEEYAYLSHRATRRQRGGSVESALINAGRRKVLCKVRKGRGRKMESWNIALQDALRVEFERLSNVGMKFSSRLIGVVARDLIRVLQSEGKLPNVNGGGVQLSTLINSGWVQRFMERHNIVSRRQCGKLMISPAKQVFIEKQVAFHLGCVKREFESGLLDEDLVDNIDETHFIINLDDGKTFGLCGAQEVKYADVTSGGQGMTMLVRLTGGRNAQIMPAFLIFQNANRSYPIRGIVDDVPGACYRTQPKGWMDRDVFLQWLQEPRAISRDPFGRTRTIFLDNCAGHGETVEQNLALEALKCSLRFLPANSTDLCQPCDSFVIQKLKDRWVSLWDAEKAELLHSPEWLQGKGAGRLPNPGKRYFLQLAARVVRDVNQMRDENGVGYARKAMIRCGLSLDLNAQWRVEQLFPHLQEIIKRHTEHFEGAPVEGLVVGAA

SEQ ID NO: 243 - protein, 342 aa, synthetic construct
MRDLSDFERGQIVGARLAGASVTKTAELFNVSRATVSTVMTAYTKHGKTSSAKRNSGRNPKLTDRDRRTLKRIVARQHKTTAAKVTAELNAHLSNTVSTKTVRRELHKANIHGRAAIAKPLITEANAKLRKKWCHDHKTWTLDDWKNVVWSDESSFTLFPTNGRVYVWRTPKEAYDPDCLLPTVKHGGGSVMIWAAISWYSAGPLIHITGRITANEYLDILNDEVLTMTSILLPNTAIFQDDNAPIHTAKKVQSWFEEHQNIIKHLPWPAQSPDLNIIEPLWGVLEQRIRSRFPSPTSLKQLADALVQEWDNIPLETIQTLYESIPRRIAAVLQANGGPTPY

SEQ ID NO: 244 - protein, 343 aa, synthetic construct
MGRKGSELCASEKESILSLSKANIKLKEISEITGRPISTICSFLKRQERRGITENKNRSGRPRKCSVQGERQLIRLVKNNRRRTLTELTNVSNENGASRLSESTVKRILRKGGYRRRLVKKKLRIREVNKKKRVNWCKQYRHKTVDDFWNNIIFSDECKVMIDGEQRVYVWRKDGEEWDPPCVAPPPGRRLDLMVWGCITAHGVGTLCIVDGNINAEKYIEIIDSNLWPVVAMHFPNNQYIFQDDNAPVHRARVVKDFVTREGITTLEWPAQSPDLNIIENCWKKMKHEINRNVHNLRTNDDLAAAVRQAWENIPLQFIQRLYQSIPRRIQAVIKSKGCLTKY

SEQ ID NO: 245 - protein, 340 aa, synthetic construct
MPKSRELTLELRAEIVTKLKAGISAAEITKLYKISRRNVYYLVKKIEEAGTLKNKKRSGRKPVLDQRQCRHLLGVVASNPSASPVKIAQESKNTIGKQISDSTIRRRLKEADFNTYVARKVIDITPTNRTIRLQFALEYIKKPLDYWFNVLWTDEAAFQYQGSYSHHFMHLKKNQKHLAAQPINRFGGGTVMFWGCLSYYGFGDLVPIEGSLNQIGYVRILNDHAFMSGNRLFPTYEWILQQDNAPCHKGRIPTKFLNDLNRSVLPWPPQSPDLNIIENVWAFIKNQRTIDKNRKRDGVIAEIDEIWSKLTLEFAQTLVRSIPKRLQAVIDAKGGVTKYY

SEQ ID NO: 246 - protein, 342 aa, synthetic construct
MTVCTPTKRARILALRDSCFGKPMSFRKISSILGLPRSTVHRNWKAVTQTGDCYAYKPRPGRPRKFSKRDARHAEIALANGSARDAADVQRQLFPNVSSRTVQRRLEEIGLHGYVRRKKPYLSSLNIKQRKVFITGTGDWDMEKWRLIVFSDESKFNLFGSDGRQYCWRRAGEAFLERNVKKVVAHRGGSLMVWGCITLKGVGRLHRIHGNLNGPKYCVILEESLLGTLHDHSLSPRSILLQHDNDTKHTSRLAKAWFDEHHILLLPWPSSSPDLNIIEHVWEELDRHLRHRPRLPRNLNELWIALQEEWYALDTDFIQNLYDSIPDRIAAVKHAHGLYTRY

SEQ ID NO: 247 - protein, 335 aa, synthetic construct
MPKGKYLSEEEKGMISAYINCNQSNRAIATKLGRNEKAIPNYRKQRLSISPPKKRGRRLKVSRRDARRIFRLAIHKRRSSREIVSDLGGVVKYSTALRVLKSSRFAKFAERKPAPAVKKHHKKARLAFAENKIGKIMFWRRIVFTDEKKFNLDGPDGYRYYWHDVRQDPELFSKRVGGGGSVMIWAGVSWHGKTELVVLEGRQNSNDYAQTLRGSLLPYLENLRAGQGVLRPIFQHDNASIHSARRTQDFLREEEVSTMVWPAKSPDLNIIENVWGVLARRVYAGGRQFDTASELKQRILKCWEEIPQDYIRTLVKSMPTRIVQVVLRRGAPIDY

SEQ ID NO: 248 - protein, 373 aa, synthetic construct
MSITSSNKMTSSVKKQTSCTSVDTVNERAQALALLDAGISVKDVAKQLCKSERWVTKWRRRREQNKQLTDQPRSGRPSKIVGVVKRKVENIKYKRGKSTRKCSKELKNSGFNVSHVTVFNYLRKVKKWKAFKRSRNQLLTKTQRQKRLKFARDHKHLTAEDWENYIFSDESSKYLFHVPNRQDDVVWGSQSDEVPDVSCVKSSAKVMIWGAMGVNGLSKLHIVPSGKTINAKYYVDKILQKELKPALNRQKNTGRIDERKLVQYPGHATFVQDGATPHTALVTQNWCKDNLPNFISKEEWPGNSPDLNCIENLWSILDSEAYKDPRPTSMDQLRRRLQRAWREIPQKHLISLIHSMPNRIKNVLKNKGGLSGY

SEQ ID NO: 249 - protein, 318 aa, synthetic construct
MPPRNRISLEQRQRIVRAFEDVREDYLAVADTIGVNRSTARGIVARYLREGRIAERPRGGANHVRVDDEMRDCLDDIINENCLLTLAQMNRELRQRLPRKPAIHDRTVARALEGMLYRVKLARPLPADRNRPDVLQKRVEYANWFMRHAVVNHSVFVDECGYNIWTARSQGRARLGERAYRQVCGQRGRNVTVALAISPTSGLVFHSAFLGGMTGQRFNDFLTQARLNLDPNEHVIFIYDGAPAHNNPAIPGPNSELKKLPPYSPFLNIVEQAISALKAAIKADISRPENQEQMNDRAEARRQGIALGNFRTQLLLHT

SEQ ID NO: 250 - protein, 383 aa, synthetic construct
MTPPPTSATPQTPPLVSREAKQFERNSAKKRVLDAFLAGQDWLGVAASDAVSVTTARRIAAKGSIEQQPRGGVRTACVKMTVEVMSKLEEYLDEQADMTMAVMKDRLLSDLSVDVSITSIHRALHGMLYTVKGLRIEKAAMNNAVNKEKRMAFAKAFNAHVAAGDMIVYHDETNFNIYLSRNQGWARVGERAVVTLPASKGKNLHVQCGVSPGNGLVLLRTHDGSVHMEENARFVADLFVAALNSDEYKECCVGKKVVVITDNAPAHSQVETMAREQLVTDGIVNSNKLVILRLAPYSPMCNAIEGCFSVLKARMKLHLAAKRRELLVRGQYNSLTANRMALMKEAVESSKEVLTQRLVWRMERHCFKSIFLAERGEDMELGK

SEQ ID NO: 251 - protein, 445 aa, synthetic construct
MPKRSKKFVLNSQSRELIIRVRDFFEREAQNGGPILPVTSVLDRVADALDISKKTIIKITKEKLGETGIEDNVLYSPNKRKRAKLTDVDNFDVDAIRRHIYNYYLRKEYPTLSNLVQTLKETGLFSGSKKSLSKVLKNIGFRYKQCNKRKVLMERSDIAMKRTDFLREAKAIDDWDNVVFLDETWLNANHTVGRSWTDDTAASASKVPSGKGARLIICHAGNACGFIDNCLLAFASKTTGDYHEEMDGEKFTQWFTNMLTSLSEPSVIIMDNAPYHSMLVDKPPSNSEKKAHLVAWLQKKGVDASMSMLKGDLIRLMNIHRSRVKRYVIDELALQHGHRVLRLPPYHCEYNAIELIWAQIKGHAARHNTEPPFSTAKMMKLLEEACNHVTKNNWKMVVNKTKAKIWDDCNRDIKVDNILDNDFELVINTNDSSSDNGDGASESDT

SEQ ID NO: 252 - protein, 470 aa, synthetic construct
MSSAAGSTSPTQEVKRRKKSVLHTQAREFVCSVRDYFEKEKDKGGPLIPVVQVVKRTAAALKISKNTVVKIGKEQYSVDCDESGTTKLHTPGKKRPRNKQVTALDDFQKDAIRRHIYSYYKRREHPTLSKLQVSLQKDDLFKGSKFSLRTVLKDIGFSYSLFNGRKILMERTDVVAWRCRFLRRIMGVEFESIVWLDETWVNASHSLRRGWNDGTPEGTMAVPVGKGGRIIVLHAGTSKGFVPNCLKMFRSKKTGDYHEEMNSVVFQEWFETSLMTNLTSPSVIVMDNAPYHSVVHDKAPTLATKKDDIIQWLKRRKVDFREDLRKAELLEIVAQKKPQFPTYVIDEIAKRHGHEIVRLPPYHCHFNAIEGVWAQIKNYIAANNKKFTISEVETLLPAAINKVTSETWAKIVNSTASAIREAAKTEGVVEECIENLIIHLGESSDSSSSAEEDNVKSDSDSDVSGVFPLQ

SEQ ID NO: 253 - protein, 343 aa, synthetic construct
MGDIPEFKRGPIVGARLAGASVTKTASLCDVSRATVSRVMSAYHQEGRTTSNRINGGRKRKLSERDVRVLTRIVSKKHKTTAAQITAELNVHLNSPVSTKTVRRELHRVNIHGRAAIAKPLVTRANAKRRFQWCKERKSWDVDNVKHVLFSDESAFTVFPTSGRVTVWRSPKEAYHPDCCMPRVKHGGGSVMVWAAISWHSLGPILVLDGRVTAKDYRTILEDHVHPMVQTLYPEGGAVYQDDNAPIHTARLVKDWFDEHESEVEHLPWPAQSPDLNIIEPLWGVLEERVRKHFPPPASRRDLATILQEEWLKIPLTTVQDLYMSFPRRIDAVLAAKGGPTPY

SEQ ID NO: 254 - protein, 339 aa, synthetic construct
MGRRTSIEKRELVLEYFKKGIPQRKIGEMVNISSSTVQHIIERFVHENRIENKGRKAPNKIFTEKESRYLVNQIRKNPTISAPKLTDMAKIDFGKICHPETVRRILRESNFNGRIAVKKPFVSAKNRIKRLQFAREHLNKSPAFWRNVLFADEVKINLFGSDGRSYVWREVNSELKPQNLRATVKHGGGNVMVWACMSSNGVGNLVFIEESMNKNIYLKLLQENLIQSATKLQIADDFRFYQDNDPKHKSALVQTWLIWQCPHIVQTPAQSPDLNVIENLWSVLKTNIRKHDIRNKFDLRNALQQEWSKISPDYTKKLVDSMPKRLHAVITNKGYPTKY

SEQ ID NO: 255 - protein, 339 aa, synthetic construct
MGAHSTVKERQLIINHHKDGKSIRQIAEMVKKSHSTIQHIIERFRKNKTVQKKIRVSPKKIFTIADERWIVNQVKKNPRISAPKLTSEIQKYLNKTVNPETVRIVLRKYKFHGRVARKKPFISKKNQKIRVEFAEEHSKKNTTFWNRVLFTDESKFNIFGSDGRCYVWRKAGEELKKKNLQPTVKHGGGGVMVWGCMSAAGTGSLHFIDGIMNKHSYIDILRTNLRESVEKLNIKDNFIFYQDNDPKHTAHLVKEWLTFNCPKVLITPPQSPDLNVIENLWSKLETEIRNHKISNKDDLKKALLEEWGKISPDYTKKLVESIPNRLTEVIRSKGLPTRY

SEQ ID NO: 256 - protein, 341 aa, synthetic construct
MPKTSELSIATREKIVLLRNQQKTYRQIADFLGLNFSTVRYVIKRYEERGSTENSERKGRPKKLTPRLKRKIRKEALKNPVISSRELAAMVQSTSGVQVSSRRIRYALNSEGLHGRVPRKKPYISETNRKKRLVFAREYVQKPLSFWENVVFSDESKFNLFGSDGRRTIWRHRNEALKKQNVLTTVKHGGGHVMVWGCVSYHGVGNLVSIEGNLNAEGYVNILRRNLRVSVEKMGIQDQYYFQQDNDPKHTARRTKEWLIYNVPRQLHTPPQSPDINIIENLWHTLDENVRKHHFTSAHHLKQLLVQEWEQINLDLIKNLVNSMPRRLQAIIDANGLFTKY

SEQ ID NO: 257 - protein, 340 aa, synthetic construct
MAKTAEISNTIRFKVIKLRNNKKSYRQIASILNMNMSTVRYIIKRYEDRGTIENKRRTGRPKVLNERQRRGIVRQVRANPFTSAGELANMVATTSQRVSESTIRNVLHLTNYYGRTARKKPFISEKNRKKRLEFAKKYSGKSIEFWENVLFTDESKYNIFGSDGRQYVWRKVNEEFKNQNIIPTVKHGGGNVMVWGCVSAKGVGKLVFIEKNMNARMYVDILRQNLGQSVRKLGMENSFLFQHDNDPKHTARVTEEWMLYNVPRRLHTPPQSPDVNIIEHIWQMLHVRIRKHTISNKDSLKKALLEEWDKISLDDIKPLICSMPNRLKAIIEAQGLNTKY

SEQ ID NO: 258 - DNA, 475 bp, synthetic construct
  source 1..475: mol_type = other DNA
tacagtaatctctctttacaagaatgacccgcaatagaatattctttttagtagaatacacggactgtttcccgtcctgggaatggccaaaacacccctattttcatgacatttattcatacacgacataagaatatggaatttcgaatgtcgccttatcccatagagtacagtgtgtactgtacaagggaacgtattttggtcctctaaaacggccaagcagatgtgcgagatctgcttggtataacggtcaaacagactttggatattcgtttgagagcgaatactgttatgttacatgacttgaatgttactcaaatcatgaaaaataacagtataacctgtgaaaatctgctctactcgagcatctcaagtgaagaaaaactgtactgtccccgccaatatttaaggagctaaactacggtacatatcttcgatcatttcatcctccattctcacatatatccgcttatcc

SEQ ID NO: 259 - DNA, 834 bp, synthetic construct
  source 1..834: mol_type = other DNA
tactgtggaatcattagaattcgtgggggccaattttcgtggattgtggttttttgtccgttcgtggggacgtaatttcgtggatctcttcgttttcatttttattaagaaagataactctggaaagtttgtttgctgacacgagttatctttctttgcgatagtacaggtacatgctgtttataccgatactaggtattaagtcttgagtaaaactcagcaataaattcgccagaaggcaaaataatctatttaaagtgtgatttttttcttggtctggtcatttttctcgaagatgtcgctgagacagtggttgactacaggtaaactcaaaacaacagcaccaacagttcctggtctgcccgaccccggggactgtacagaacctcgggaagcggcagcattccaagctgcaaatgatgcagttgaaactattgttacaagtactaccgatagtcctctctcgacacctaggaaaagaaagcggggtgaatataacaacctggacgaagaacaaagggctaaaattggcagactcgctgttgaagacggtgttgcaagggcaacaaggcgctttatgtccaattctggtattaaagtgtcagaatcaacagtaagaagcatccgagaccggtatctaagagcaagaaaagcatctggtatgtcatagtatttatataacttttatataacgtaaatattaaattacattaagcattgcggcacttttttccgtaataaataaatataaatacgcaataaatatgatgcattaatttttaaggggattcacatctcgctgctcttcccagatcccctcgaggaaacactttg

SEQ ID NO: 260 - DNA, 101 bp, synthetic construct
  source 1..101: mol_type = other DNA
tacagtgggtgatcacctgacgatcgttcccacctgacgatcgattttcccctcaccgatatttcaaccaccaaacgtcaaactcttgtatctcattcaat

SEQ ID NO: 261 - DNA, 219 bp, synthetic construct
  source 1..219: mol_type = other DNA
taacagttcggcaactattttggcaaacataaattattttggcaatgcactgctaccctttttgccgcacttttccgaaacagaatcacccgaaccgcccccgccacttgctaactctgtaactcagtacaacgattacaaactgcccgccttttacggttttggcgccatgccaaagcccgttattaccgcgtgtcggccaaaaattcaggctgtggc

SEQ ID NO: 262 - DNA, 622 bp, synthetic construct
  source 1..622: mol_type = other DNA
tacaccgaatagggtaattagtctagtctacagtatttccgtacggggtttttcgaggaacacatcattattggtgcagcctacaccatttgaaatactacactatttacaagttgtacgaaaataatgaagtctacactatttcaaaacgtgctaaatagtgaagcataaatgacacgtcattgtcttcatattttcgaaagttcgaaaatacgggcttttaaacgctgttacgttttgcacaatttgaaattcacagcaagcagggcattgtgggtaagaatcagtcctcacaggcatagttgagacttttatcgtctcaacgactatattgtactggcagcctgaagcgtcacacgaacgttcttcgggaagctgtgtacgtcaaacaaggtccttagcaccctctttcacgaacaacaacatagtgagctgaccataatgaagccgttacctcatctacgtgttcactgtagcaactgaacggcttaatagctgttctttattcgtaagctgaggtgataaaagttagcaaccttgcctccaaaacgtataagaatcactgtagcattaccactctttgtgtctgtcccatcgagaacccgagacaacatcctgcaat

SEQ ID NO: 263 - DNA, 189 bp, synthetic construct
  source 1..189: mol_type = other DNA
tacaggggttggacaaaataatatgaataccatgcaatatagtacagtttgttgcatgttggcaacactgcttccgtgtaaataatgcgacgcgtatgtaatgcgcagtttacgtggcaggtcttgtaggttatatttcagtgtattatacgctttgttctgcgacggttataacaagcgatcggtgaa

SEQ ID NO: 264 - DNA, 210 bp, synthetic construct
  source 1..210: mol_type = other DNA
tacagtcttgggtcaaaagtattcgtaaaaaatggcggccgtcattttcactaccgcgcttcgctccccatttcagcgtcgttattgacgcacaaactagagttatccctcttacttggtacaaatatacacaagcacatgacggatcacgtggctgtgttatgacgtattttgtcgtctgcaaatgttttgtttccgctatcaaaacaa

SEQ ID NO: 265 - DNA, 380 bp, synthetic construct
  source 1..380: mol_type = other DNA
tacaaccatagtcaaaattattttcacaacacacaattatttgcacatcccaacttcttttagttgttattcttgttgttgttgaacgatttcactgaaacaaatgttatcgcgtagcttgatcctcgctgaagggacacaaaaagaaatgggaaagatagaggttcataggacaaatctaaaagcaaaagaaaaaaagcttatcagttgtcagaagtatttgcacacgttttcggtgcgtcaaaattattttcacaatgcaatttcgtcattagtatttggttttcagcgacaagtggcggttcgtttgctctaataaattcaattcggtgaaaattcaataaaaataataatttatcttttacatttgtctaaacaaa

SEQ ID NO: 266 - DNA, 323 bp, synthetic construct
  source 1..323: mol_type = other DNA
tacagtggctgccaaaaataatgggacactggtccgggcgcctttgtccgtccccttgtaaccggactacgtacttgtatggtcgcgcctgtatatcaatgtatagattccaacccttggtatatgtctgcgataatttcgacggcgcgcgcaggatgcgcgagggcatgagggtgccgcgaccgtgagatggctttagcgcccattgttctttcaaagctgtggtcgtgactctttcctccatatcacgacttaacgaaccccaattgctgaacgactctcatattcatcttcttaatctcttaatctgtctattaaacaca

SEQ ID NO: 267 - DNA, 326 bp, synthetic construct
  source 1..326: mol_type = other DNA
tacagtctggtgcaaaattatagccgcacccgaggttcttgagagtgacgacgactgaattgtttccgtatgaggctgttgcggcttttgaggcctgtgtgatggaagggaacgatagcggaaatttcgacttgtaatggttaatacttatcgagtaactaccagaagtcgttttggcatctggtgcaaaattatagccgcaccaaactattagctgattttttcaaaactaattagtccatttttgtaattattgactgtatgaaaaacagacacaccctcggagtcatttggcgccatttcgcgccttaagcactcgggctgac

SEQ ID NO: 268 - DNA, 176 bp, synthetic construct
  source 1..176: mol_type = other DNA
taaagggtgttaagaaagttcgttctggttgtaaattgacaaaaataaattgataaaagcaatatttctgtttcaaattcacactggttatactagagaattttctctcaacatatcatcagttatcctcccctatgtgcactctctggtggtgtcatagagtgatgtaaacattt

SEQ ID NO: 269 - DNA, 643 bp, synthetic construct
  source 1..643: mol_type = other DNA
tacaccttttgctgttcatttacgcaaaatatacattcattttcgcgaagtgtagaatcataacgcgaatatacattcattaacacttccataaacttcattaacgcgaaggaactttcaatagcgctatttgcatgtgtataaacgttcaatagcgtcaacgaacaaacaagagcacctttcgacaatcaaggacgacaaatgtactttcaatctcgcgcaatggtcaatcattaacaccaaaagaaaatctataacatactccgacaatcaacggcgtattagagacattcaataatgcaatttgcatagagacgcacaatcaattgcacctacatacaatcgttcattggaaatggtcaatcaataacgcgaactgaacaaacgttggaatctttcgtaatgaacaataggacgaaaagatattcattagtgtaattcacgcccagttgcaaatctgaaaatactgaacaataaaatacaagaatatgtacattatcatttaaattttatgttaacatttttggcacaatctttcgctgagacagctataaaatgaggaacatctttgaacgtagtgccgagcgcgaagtactgtaactgtacatgtacacgcattgagacagaaagaaccgagaaaatc

SEQ ID NO: 270 - DNA, 238 bp, synthetic construct
  source 1..238: mol_type = other DNA
taccaggcaagttgagcactttcacaagcaaagcgatcaatcgaggttttattaattcacctaaaataaggttttacagccaaagcggtttggcgccatattttttccaaccaaagtggtgcggaagcaacgtgagccactcgatactggcgaagccgcccatggtctccatgcagacactccttcatttgcgtgttcgcctcctcccccccaagcctccccaagcagctctccaaca

SEQ ID NO: 271 - DNA, 603 bp, synthetic construct
  source 1..603: mol_type = other DNA
taatagaacgcggctatgaagttaatcctgacacattgcgcaactatgaagtttctgaataatgacaatgtcaaccacataggaaaaaattacataattacttattgcgtgtgacagaatagtgagtggcaataatatcacagacaatatatcgatacacgttaaaaagttcgtatagctaaaaatattgttatgttttatgatgatgtgcattattgtaaataaaacgtaaaattagatatattagtaatattctccttcagttaattataaatattaatgaataaatgagtttgtttctttattactaacctctgggaattgaactctttttacctagagaaaactttttgaatttgaatgtaaagtacaatatttcctgtcaattaattcaatgacacgtcaataggtatgataaaacaattttaagtattgccttgatcgcttttccaaaggttgatacttttgattgtgctaaacttgtgcatgtttttggctattaatacctagtgccatcccccatctcacccattgaccaaggggcagattgaagcaagggcttgaacttggaatttgaaatgcttactacttggaattgttaaa

SEQ ID NO: 272 - DNA, 271 bp, synthetic construct
  source 1..271: mol_type = other DNA
tagtctgggagacgagtctttggtattgacagctcggtagcgtctttccgttgcctagcgaccgcaggcaggcagccaatgacggcctgactttgagcgggtagcaagggccacgttgccgctctgaaacccggtcgcgcgcgcttatgaaacttaccagtgtctcgcgtgcaggcggtgcggtcgttcgtcgtttgtctgaagttgaattcgcagtttatttcgtttttgttgtttttagtgtttctttgtttatgtttcgttgtaaaca

SEQ ID NO: 273 - DNA, 188 bp, synthetic construct
  source 1..188: mol_type = other DNA
tacaggggttggacaatgaaactgaaacacctctcattttagtgtgggaggtttcatggctaaattggaccagcctggtggccaatcttcattaattgcacattgaaccaataagagcagactgtgaaggtccaattagcagggtaagagcacagttttgctcaaaatattgcaatgcacacaacatt

SEQ ID NO: 274 - DNA, 352 bp, synthetic construct
  source 1..352: mol_type = other DNA
tacagtggctcacagcttatttgatgcaagcaaaaattttttgaaaaatgttgtttatttcaaaatattttgcaaaattaaaaaaacaaagtatgcagagttattgtttatagtgtgtttattcatttaaaaaaaaaaaaacccaaaaatcttcttccattccttagtaatgcatcaaaaaccaaaaccaaatgaaaaattcatgtcacagcttaaatgatgcagttaagaataccgttatttttgtcgccagattacacagaaattaaaatatttttattctgcgatcatttgtaatctagtttttgagcgaaaatattataaaaattaaagtgagtgcattttgttaacc

SEQ ID NO: 275 - DNA, 351 bp, synthetic construct
  source 1..351: mol_type = other DNA
tacagtggctcaaagtgaaattgatacacccaagcaaaaatgtttcaaccttagttttattcccctaattattaaaaaaaattaaatcactagttatttttattaacttacaaacactttattgttataaataactagagaaataaaaattaaatacttaacaaaaattaccgattaaattataacatttagttcaaagttacatcaaaatgaaaatgatacaccctactgtttaccttttaatctgacctttgactcatctattcattaacattgatatttgtatactcggaaggaataatccttttagttttgattcagatttcattctgttaagtctaaaatttcaca

SEQ ID NO: 276 - DNA, 350 bp, synthetic construct
  source 1..350: mol_type = other DNA
tacagtgatggaaaaaagtatttgcacatcatgtttttataaataaatgagttcaaggaaacaaaaaacttcgctttcctttgcaaaaatgaaaagtgcgtttattaaaaactggaaaaaaccttttgtaaaaaaatgacaacacttcataatgaaacaaaaaaattaaaacaataaaagtgcttggaaaaaagtatttgcacacttggtctactttgactcacaagttttgtttacttttgcattaatcctcctgtgtccataggtttgtttactaatttatctctatcgtttattaccagtattctctagtctttcaaacagttttcttcaatcaatagacaattatc

SEQ ID NO: 277 - DNA, 314 bp, synthetic construct
  source 1..314: mol_type = other DNA
tacagtggctacaaaaagtatttgcacacctacgatttttgttatttccaaaaggcaaatgaaataaattatatcgtactgtcttatatttttttgttagtcattttagatacatattttatagaaaaaattatttaattatatttatgtttgccaaaaaaaattaaaaactacattttaaaacacaaaaagtatttgcacaccaaacaatattcttgtttgtttaattaataaatccgttcaaaggttgatttaaaattttaaattagaaataattatgttttggttccaattcagttgttagcaagattaac

SEQ ID NO: 278 - DNA, 80 bp, synthetic construct
  source 1..80: mol_type = other DNA
ctgccggcataggaatatactctttacatgaataataatgtcctttcccgtcctcattcttataaagagagattactgta

SEQ ID NO: 279 - DNA, 339 bp, synthetic construct
  source 1..339: mol_type = other DNA
ttagtcgatctattattctactgagtagtaattaagattgaactgaaatattaaatgttataaataaaacttttcttcttcacactgcgttctaatattttttgctgtaaaatacaaacgttatctttaagagtactctgcgttctttgcattgaaatatgaacatgaaaattaaaaatgccttcgtacttctactttatgcgtacatatagagatttgtttatttaaaaaagtacaaattcagtgtctttattcgttgaggatgtaaattcgtggggaaggggtacccacgaaatccacgaaaattgagccaccacgaattctaatgattccacagta

SEQ ID NO: 280 - DNA, 159 bp, synthetic construct
  source 1..159: mol_type = other DNA
aattgacagaactactctaaattattactatattatgccgccaaaaatttgagtgataatattagttgtatgtggttgaattgcttcatgtttgttgtgctggtggtggttgaaatcgatcgtcaggtgggaacgatcgtcaggtgatcaaccactgta

SEQ ID NO: 281 - DNA, 366 bp, synthetic construct
  source 1..366: mol_type = other DNA
tggtggcgaaggttttgtcgttgagcacagtgtcaactgtgtctctctcagattttcagggacagtgacgtgtacagtcccagaacgttgaataaagtgattttttgggccacggcgattatttgaagttgccagattagttcccatgcgattcggacctgtaaaaggggttcatttaagtgacttcatttccttgaatgaacacatgttgtgatttcgcaaccgtatgtaatgctagatgtgccttttctgtgtttattgagcttgcaactgtcaaaatcggtgaagggcttggtaagatacggccttttcaccagacatgcccagaaaaaaaggtcacttgccacattttatcatgaactgtta

SEQ ID NO: 282 - DNA, 210 bp, synthetic construct
  source 1..210: mol_type = other DNA
tatacgttctacagtttaagagccttgctacattttctcttagattttaatgaaaaataaaggttacatcaattgcagtctgttcctttatagtgaagccaacactatttgttttttgggtggaatttgacgcagtcagaatactgtagcctgcactaattcagatgcttcactatttcagcaccaacactaattaccctattcggtgta

SEQ ID NO: 283 - DNA, 55 bp, synthetic construct
  source 1..55: mol_type = other DNA
taaataaatattgtgtatatgtcaggtgttcatattattttgtccaacccctgta

SEQ ID NO: 284 - DNA, 112 bp, synthetic construct
  source 1..112: mol_type = other DNA
ggttggtgcaaatttaattaagttaaaaagttgatgaaactaaggtgacctagatttgtttacatcgccgccattttgttttttttacgaatacttatgacccgagactgta

SEQ ID NO: 285 - DNA, 334 bp, synthetic construct
  source 1..334: mol_type = other DNA
tgtattttgataaaattaagaaaatcttttgttaaaattagatattatattaagctgaaataaatcaaactaacttttgtgaatataattttgacgcaccgaaaacgcgtgcaaatacttctgacaactgataatctttttttcttttgcttttagatttttcctatgaacctctatctttcccatttctttttgtgtcccttcagcaaggatcaagctacgcaataacatttgtttcagtgaaatcgttcaacaacaacaagaataacaaataaaagaagttgggatgtgcaaataattgtgtgttgtgaaaataattatgactagggttgta

SEQ ID NO: 286 - DNA, 269 bp, synthetic construct
  source 1..269: mol_type = other DNA
ctgcaatttcgttttctcgtggacttggaatgtggttttgtcacttgttcctgcgtataggacatacccatttcggtatgcttccccttgcccccgctttttcccagaataattctatacacttttgttgattattctacgtacatatagtacaggtcctatactatgaatgcacacactcattggccattgtagggctcgaacagagctgcagcatcgtcgtttaggcgccggtagcgcgcgtcccattatttttggcagccactgta

SEQ ID NO: 287 - DNA, 218 bp, synthetic construct
  source 1..218: mol_type = other DNA
tttgaattgaaggtagtgcggctataattctgcaccagtccaaaaaaagcttttttcgtgtgtcttaagaatgcaactttcttctgcaaaatccagcggcatattactttaaaccatcatacgcgttacaacaaagaccgtaaacaactccgaaaataactttttcatctcaatctataaacttttcgggtgcggctataattttgcaccagactgta

SEQ ID NO: 288 - DNA, 84 bp, synthetic construct
  source 1..84: mol_type = other DNA
aaatcaaaaaacttacaaacgagttgtttattaatgaatctaggctaaagtaaatcacggaacgaactttcttaacacccttta

SEQ ID NO: 289 - DNA, 583 bp, synthetic construct
  source 1..583: mol_type = other DNA
cactatcatgggtgctaaatgtgtcagagaaatattggcactatcacggccgccaaatgtggtcagtggtacagacttatgcagacgtacattccaagatgcttaaatagtgaagaaattgaaggataatagccttaaactaagaaacagttttgaactttcgttttccttagttgattgttattacttcacaacttgtttgtcagttcacgttaatgattgaccattttgaataaacgattgtatgaaggtgcaattgattgtgcgtctctatgcaaattgaattattgtatgtctcaaatacgccgttgattgtcaaagtatgcatagattttctattggtgttaatgattgaccattgcgcgagattgaaagtacatttgttgtctttgattgtccaaaggtgcaattgttttgtccgttgatgcaattgaatgttaatacatatgcaaatagcgttattgaaagttcgttcacgttaatgaagtttatggaagtgttaatgaatgtatatttgcgttatagtttcaactgttgacgtaaatgattgtatattttgcgtaaatgaacagcaaaaggtgta

SEQ ID NO: 290 - DNA, 141 bp, synthetic construct
  source 1..141: mol_type = other DNA
ttttgatataaaccactttgcatgggaaaaatatatcagaaaaccacttcgcctgacaagcgtccttttttcgttaaattattaagttgcacaagctggccaaatccgttttgcttgtgaaagtgctcaacttgcctggta

SEQ ID NO: 291 - DNA, 226 bp, synthetic construct
  source 1..226: mol_type = other DNA
taaagtaaagaatataaatattatctctaccatagaagtattaagtaattaaactacttctatgatctctacctagatgttttttttttcactttcttcatatacttacatctgataactgcattcgactttactcacacttgacgaattttatcgatacagtgatcgaacgtataacaaccctagaggcatgtgtcaggattgacttcatagccgcgttctatta

SEQ ID NO: 292 - DNA, 481 bp, synthetic construct
  source 1..481: mol_type = other DNA
ctacaacgtattcaggaatgtaaggagggagtttgctatcgatctacaaccagatcatcatattgtgagtactttcttcagattataactcttaatacactgaccaattattctgtagcttgtagtagaacaaattaataatgctaatacttaacaatggaaaccaaaactgctaatgttcaacaacttgcgaaaatagttttcatttcagttgtgaagtttaacaaataactttattatgtttcagcatcttagatacttgtactaaatagctcttatcagttttcgagttaatatatttcaagcatcaactttaaataaattcacgcgtaccaatacgacttgtattttgtctcgcttttatttctgctgctagagaatgcgtgtagcagacagggcgccgcgtgctgtgagccacgttcggcaacagcgccgtctgcccgccggaactgtcagtaccaatcactcgtctcacggacta

SEQ ID NO: 293 - DNA, 54 bp, synthetic construct
  source 1..54: mol_type = other DNA
taaagtattgtggtccaaaaccaggtgtttcagtttcattgtccaacccctgta

SEQ ID NO: 294 - DNA, 273 bp, synthetic construct
  source 1..273: mol_type = other DNA
gtacttagaattagtccggaagtttgtaaaaatattagtttttttgatgttgcatcatttaagctgtgacatgaatttttcatttggttttggtttttgatgcattactaaggaatggaagaagatttttgggttttttttttttaaatgaataaacacactataaacaataactctgcatactttgtttttttaattttgcaaaatattttgaaataaacaacatttttcaaaaaatttttgcttgcatcaaataagctgtgagccactgta

SEQ ID NO: 295 - DNA, 274 bp, synthetic construct
  source 1..274: mol_type = other DNA
gatgtgaagaaaaggtttaaatacatagtatgtatgtaagtgataagaggtgtatcattttcattttgatgtaacttttaagtaaattttataatttaacacataatttttgtgtaagtacttaatttctatttctgttgttatttataataataaagtgattataagttaataaaaataaatagtgttttaatttcttttaataactatgggattaaaattaaggttgaaacattttttcttgggtgtatcaatttcactttgagccagtgta

SEQ ID NO: 296 - DNA, 243 bp, synthetic construct
  source 1..243: mol_type = other DNA
aactattaaaattgagttttaacttgtaaatgtggaagtgtgcaaatacttttttccaagcacttttattgttttaattattttgtttgattatcaagtgtttttatttttttgtaaaagggttttttcagtttttaataaatgttcttttcatttttgcaaaggaaagcgaagtttttttgtttccttgaactcatttattaataaacatatgatgtgcaaatacttttttccatcactgta

SEQ ID NO: 297 - DNA, 241 bp, synthetic construct
  source 1..241: mol_type = other DNA
aattaatttttttattaaattttatatagaatcaagatgtgtgcaaatactttttgtgttttaaaatgtagtttttaattttttttggcaaacataaatataattaaataattttttctataaaatatgtatcaaaaatgactaataaaaagatataagacagtacgatataatttatttcatttgccttttggaaataacaaaaatcgtaggtgtgcaaatactttttgtagccacagta

SEQ ID NO: 298 - DNA, 584 bp, synthetic construct
  source 1..584: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 299 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 300 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 301 - DNA, 600 bp, synthetic construct
  source 1..600: mol_type = other DNA
atggggaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcc

SEQ ID NO: 302 - DNA, 500 bp, synthetic construct
  source 1..500: mol_type = other DNA
gggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcg

SEQ ID NO: 303 - DNA, 57 bp, synthetic construct
  source 1..57: mol_type = other DNA
gccactaacttctccctgttgaaacaagcaggggatgtcgaagagaatcccgggcca